(19) United States
(12) Patent Application Publication
(10) Pub. No.: US 2024/0376222 A1
(43) Pub. Date: Nov. 14, 2024

(54) TFR ANTIGEN BINDING PROTEINS AND USES THEREOF

(71) Applicant: THE BOARD OF REGENTS OF THE UNIVERSITY OF TEXAS SYSTEM, Austin, TX (US)

(72) Inventors: Zhiqiang AN, Houston, TX (US); Ningyan ZHANG, Houston, TX (US); Peng ZHAO, Houston, TX (US)

(21) Appl. No.: 18/691,125

(22) PCT Filed: Sep. 13, 2022
(86) PCT No.: PCT/US2022/076380

§ 371 Date: Mar. 12, 2024

Related U.S. Application Data
(60) Provisional application No. 63/243,453, filed on Sep. 13, 2021.
(60) Provisional application No. 63/320,188, filed on Mar. 15, 2022.
(60) Provisional application No. 63/399,199, filed on Aug. 18, 2022.

(51) Int. Cl. C07K 16/28; A61K 49/00; C07K 14/725

(52) U.S. Cl. CPC C07K 16/2881; A61K 49/0002; C07K 14/7051; C07K 2317/20; C07K 2317/21; C07K 2317/24; C07K 2317/35; C07K 2317/569; C07K 2317/622

(57) ABSTRACT

The present disclosure is generally directed to compositions that include antibodies, e.g., monoclonal, antibodies, antibody fragments, etc., that specifically bind a TfR protein, e.g., a mammalian TfR or human TfR, and use of such compositions in preventing, reducing risk, or treating an individual in need thereof.

### CROSS-REFERENCE TO RELATED APPLICATIONS

This application is a national phase application under 35 U.S.C. § 371 of International Application No. PCT/US2022/076380, filed Sep. 13, 2022, which claims the benefit of priority to U.S. Provisional Application Nos. 63/243,453, filed Sep. 13, 2021; 63/320,188, filed Mar. 15, 2022; and 63/399,199, filed Aug. 18, 2022. The contents of each of the referenced applications are hereby incorporated by reference in their entirety.

### REFERENCE TO A SEQUENCE LISTING SUBMITTED VIA ELECTRONIC FILING

The content of the XML file of the sequence listing named “UTSHP0292US_SEQLIST.XML” which is 377,661 bytes in size was created on Mar. 11, 2024, and electronically submitted to the USPTO's Patent Center herewith the present application is incorporated by reference in its entirety.

### TECHNICAL FIELD

The present disclosure relates generally to transferrin receptor (hereinafter, TfR) and relates particularly to anti-TfR antibodies and therapeutic and diagnostic uses of such antibodies.

### BACKGROUND

Transferrin receptor is a membrane glycoprotein expressed in all human nucleated cells and that mediates cellular uptake of iron through binding with the plasma glycoprotein transferrin. Tfr can cross the blood brain barrier (BBB) and mediate delivery of transferrin inside brain tissues.

TfR receptors mediate cellular uptake of iron from the plasma glycoprotein transferrin via receptor-mediated endocytosis of ligand-occupied TfR into specialized endosomes. Acidification within the endosomes leads to iron release. TfRs are involved in the development of erythrocytes and the nervous system and can positively regulate T and B cell proliferation through iron uptake.

### SUMMARY

The present disclosure is generally directed to compositions that include antibodies, e.g., monoclonal, chimeric, humanized antibodies, antibody fragments, etc., and conjugates of any of the foregoing, that specifically bind a TfR protein, e.g., a mammalian TfR (e.g., any non-human mammal) or human TfR, and to methods of using such compositions.

The investigators have created and characterized certain monoclonal antibodies with binding specificity to TfR, a receptor protein associated inter alia with transport of a protein (e.g., transferrin) across the blood brain barrier in both humans and primates. The antibodies bind to TfR noncompetitively with transferrin, therefore having no effects on transferrin binding and delivery into cells. In addition, the anti-TfR monoclonal antibodies can be fused to therapeutic or diagnostic peptides or conjugated with therapeutic or diagnostic small molecules. The present disclosure provides polypeptides with affinity to TfR, polynucleotides that encode the polypeptides, methods of producing the polypeptides, and methods of treating human conditions.

Additional details of these and other embodiments are set forth in the accompanying drawings and the description below. Other features, objects and advantages will become apparent from the description, and from the accompanying claims.

In one aspect, the present disclosure provides an isolated monoclonal antibody, or an antigen-binding fragment thereof, wherein the antibody specifically binds to TfR and wherein the antibody competes for binding of the TfR epitopes with an antibody selected from the group consisting of 1B2, 1C8, 2C3, 3H8, 4G1, 5B6, 7A1, 7B10, 8A5 or 8G5.

In another aspect, the present disclosure provides a recombinant polypeptide comprising an antibody VL domain (shown in Table 3) comprising CDRs 1-3 of the VL domain of 1B2; CDRs 1-3 of the V_{L} domain of 1C8; CDRs 1-3 of the V_{L} domain of 203; CDRs 1-3 of the V_{L} domain of 3H8; CDRs 1-3 of the V_{L} domain of 4G1; CDRs 1-3 of the V_{L} domain of 5B6; CDRs 1-3 of the V_{L} domain of 7A1; CDRs 1-3 of the V_{L} domain of 7B10; or CDRs 1-3 of the V_{L} domain of 8A5 or CDRs 1-3 of the V_{L} domain of 8G5.

In yet another aspect, the present disclosure provides a recombinant polypeptide comprising an antibody V_{H} domain (shown in Table 2) comprising CDRs 1-3 of the V_{H} domain of 1B2; CDRs 1-3 of the V_{H} domain of 1C8); CDRs 1-3 of the V_{H} domain of 2C3 CDRs 1-3 of the V_{H} domain of 3H8; CDRs 1-3 of the V_{H} domain of 4G1; CDRs 1-3 of the V_{H} domain of 5B6; CDRs 1-3 of the V_{H} domain of 7A1; CDRs 1-3 of the V_{H} domain of 7B10); CDRs 1-3 of the V_{H} domain of 8A5 or CDRs 1-3 of the V_{L} domain of 8G5.

In yet another aspect, the present disclosure provides a host cell comprising a polynucleotide molecule encoding the polypeptide of any one of the above embodiments.

Additional details of these and other embodiments are set forth in the accompanying drawings and the description below. Other features, objects and advantages will become apparent from the description, and from the accompanying claims.

The numbers, E5, E6, and E7 and the like are used interchangeably herein with 10⁵, 10⁶, and 10⁷, respectively, and the like.

The term “comprising” and variations thereof (e.g., comprises, includes, etc.) do not have a limiting meaning where these terms appear in the description and claims.

As used herein, “a”, “an”, “the”, “at least one”, and “one or more” are used interchangeably, unless the context clearly dictates otherwise.

Also herein, the recitations of numerical ranges by endpoints include all numbers subsumed within that range (e.g., 500 to 7000 nm includes 500, 530, 551, 575, 583, 592, 600, 620, 650, 700, etc.).

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

An “agonist” antibody or an “activating” antibody is an antibody that induces (e.g., increases) one or more activities or functions of the antigen after the antibody binds the antigen.

An “antagonist” antibody or a “blocking” antibody is an antibody that reduces or eliminates (e.g., decreases) antigen binding to one or more ligand after the antibody binds the antigen, and/or that reduces or eliminates (e.g., decreases) one or more activities or functions of the antigen after the antibody binds the antigen. In some embodiments, antagonist antibodies, or blocking antibodies substantially or completely inhibit antigen binding to one or more ligand and/or one or more activities or functions of the antigen.

As used herein, “percent (%) amino acid sequence identity” and “homology” with respect to a peptide, polypeptide or antibody sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms known in the art needed to achieve maximal alignment over the full-length of the sequences being compared.

An “isolated” nucleic acid molecule encoding an antibody, such as an anti-TfR antibody of the present disclosure, is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies herein is in a form other than in the form or setting in which it is found in nature. Isolated nucleic acid molecules therefore are distinguished from nucleic acid encoding the polypeptides and antibodies herein existing naturally in cells.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

The term “isolated molecule” (where the molecule is, for example, a polypeptide, a polynucleotide, or an antibody) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Accordingly, an anti-TfR antibody of the present disclosure, is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies herein is in a form other than in the form or setting in which it is found in nature. Isolated nucleic acid molecules therefore are distinguished from nucleic acid encoding the polypeptides and antibodies herein existing naturally in cells. Moreover, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

The term “epitope” refers to that portion of a molecule capable of being recognized by and bound by an antibody molecule, or antigen-binding portion thereof, at one or more of the antibody molecule's antigen-binding regions. Epitopes can consist of defined regions of primary secondary or tertiary protein structure and includes combinations of secondary structural units or structural domains of the target recognized by the antigen binding regions of the antibody, or antigen-binding portion thereof. Epitopes can likewise consist of a defined chemically active surface grouping of molecules such as amino acids or sugar side chains and have specific three-dimensional structural characteristics as well as specific charge characteristics. The term “antigenic epitope” as used herein, is defined as a portion of a polypeptide to which an antibody molecule can specifically bind as determined by any method well known in the art, for example, by conventional immunoassays, antibody competitive binding assays or by x-ray crystallography or related structural determination methods (for example NMR).

The term “binding affinity” or “K_{D}” refers to the dissociation rate of a particular antigen-antibody interaction. The K_{D} is the ratio of the rate of dissociation, also called the “off-rate (k_{off})”, to the association rate, or “on-rate (kₒₙ)”. Thus, K_{D} equals k_{off}/kₒₙ and is expressed as a molar concentration (M). It follows that the smaller the K_{D}, the stronger the affinity of binding. Therefore, a K_{D} of 1 □M indicates weak binding affinity compared to a K_{D} of 1 nM. K_{D} values for antibodies can be determined using methods well established in the art. One method for determining the K_{D} of an antibody is by using surface plasmon resonance (SPR), typically using a biosensor system such as a Biacore® system.

The phrase “effective amount” or “therapeutically effective amount” as used herein refers to an amount necessary (at dosages and for periods of time and for the means of administration) to achieve the desired therapeutic result. An effective amount is at least the minimal amount, but less than a toxic amount, of an active agent which is necessary to impart therapeutic benefit to a subject.

The term “inhibit” or “neutralize” as used herein with respect to bioactivity of an antibody molecule of the invention means the ability of the antibody to substantially antagonize, prohibit, prevent, restrain, slow, disrupt, eliminate, stop, reduce or reverse for example progression or severity of that which is being inhibited including, but not limited to, a biological activity or binding interaction of the antibody molecule to TfR.

As used herein, “vector” means a construct, which is capable of delivering, and, preferably, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

The term “treating”, as used herein, unless otherwise indicated, means reversing, alleviating, inhibiting the progress of, delaying the progression of, delaying the onset of, or preventing the disorder or condition to which such term applies, or one or more symptoms of such disorder or condition. The term “treatment”, as used herein, unless otherwise indicated, refers to the act of treating as defined above. The term “treating” also includes adjuvant and neoadjuvant treatment of a subject. For the avoidance of doubt, reference herein to “treatment” includes reference to curative, palliative and prophylactic treatment. For the avoidance of doubt, references herein to “treatment” also include references to curative, palliative and prophylactic treatment.

The term “comprising” and variations thereof (e.g., comprises, includes, etc.) do not have a limiting meaning where these terms appear in the description and claims.

All scientific and technical terms used herein have meanings commonly used in the art unless otherwise specified. The definitions provided herein are to facilitate understanding of certain terms used frequently in this application and are not meant to exclude a reasonable interpretation of those terms in the context of the present disclosure.

It is understood that aspect and embodiments of the present disclosure described herein include “comprising”, “consisting,” and “consisting essentially of” aspects and embodiments.

Unless otherwise indicated, all numbers in the description and the claims expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviations found in their respective testing measurements.

The above summary of the present invention is not intended to describe each disclosed embodiment or every implementation of the present invention. The description that follows more particularly exemplifies illustrative embodiments.

### BRIEF DESCRIPTION OF DRAWINGS

The present disclosure describes monoclonal antibodies, and fragments thereof, with binding affinity to human transferrin receptor (TfR) antibodies whose characteristics, affinity and kinetic binding property determined using Bio-Layer Interferometry (BLI) are described in the figures.

FIG. 1. Illustrates the Kinetics and binding affinities of human transferrin receptor (TfR) antibodies and represents the kinetic binding graphs determined using BLI method with Octet 96-Red instrument. Protein A sensors (Fortebio) were used to capture anti-TfR IgG. During all incubation steps, sample temperature was set to room temperature with 1000 rpm shaking. Individual purified TfR antibodies were loaded onto sensors. After antibody capture, the antibody-loaded sensors were subject to a solution flow containing huTfR-ECD-His at designated concentrations: (indicated by the colored lines under each graphs) and for a defined time (shown in X-axis). After binding, the sensors were incubated in blank kinetics buffer to allow free dissociation of TfR for designated time. The binding kinetics parameters were calculated by the software from Fortebio (version 11) using 1:1 binding model with global fitting. KD is calculated by kdis/kon and is presented in the table

FIG. 2. Illustrates that TfR monoclonal antibodies do not compete with transferrin (TF), a natural ligand for TfR. Using the BLI method with Octet 96-Red instrument. Purified candidate TfR antibodies were loaded on Protein A sensors (Fortebio). Individual purified TfR antibodies were loaded onto sensors and after antibody capture, the antibody-loaded sensors were subject to a binding solution containing huTfR-ECD at designated concentrations for a defined time. The TF competition binding assay comprised huTfR-ECD mixed with a saturating concentration of huTF (10× of huTfR ECD).

FIG. 3. Illustrates the Kinetics and binding affinities of murine transferrin receptor (TfR) antibodies and represents the kinetic binding graphs determined using BLI method with Octet 96-Red instrument. Protein A sensors (Fortebio) were used to capture anti-TfR IgG. Individual purified TfR antibodies were loaded onto Protein A sensors and subject to a solution flow containing muTfR-ECD-His at designated concentrations: (indicated by the colored lines under each graphs) and for a defined time (shown in X-axis). After binding, the sensors were incubated in blank kinetics buffer to allow free dissociation of TfR for designated time.

FIG. 4: Illustrates the binding kinetics parameters were calculated by the software from Fortebio (version 11) using 1:1 binding model with global fitting. KD is calculated by kdis/kon as determined from the FIG. 3 data.

FIG. 5: Illustrates increased presence of TfR monoclonal antibodies in brain tissue in comparison with the isotype control antibody. To demonstrate the ability of the TfR monoclonal antibodies to transit the blood brain barrier, following IP injection of antibodies, a serum sample was collected, then the mouse brain was harvested and perfused with DPBS. The antibody concentration in brain lysate and serum was determined using sandwich ELISA in which a bispecific antibody was captured on a plate-coated anti-human Fc. The amount of antibody was then quantified using anti-human Fab-HRP. As seen in the graph the injection of TfR antibody resulted in the detection of more significantly more brain penetration than did the injection of a control antibody

FIG. 6. Illustrates that TfR monoclonal antibody facilitates transit of a fused protein into the brain. The diagram illustrates the TfR antibody fusion protein construction using knob/hole pairing in Fc. The fusion protein sequences used in the example are from the R1D2 and R2D3 domains of VEGFRs and the sequences were fused to human Fc fragment at upper hinge region. Anti TfR antibody Fab and a control IgG Fab were fused to the C-terminus of the Fc using a 3×GGGS linker as illustrated in graphic. The confocal images demonstrate the detection of TfR antibody fusion protein in mouse brain parenchyma, but not that of isotype control antibody fusion protein using immunofluorescence staining. Twenty-four hours after intraperitoneal (ip) injection of the mice with either of the antibody fusion proteins or control, the mouse brains were harvested and perfused with DPBS and fixed in PFA. The confocal images represent 40 μm floating brain tissue sections that were stained overnight with fluorescent anti-human IgG and TO-PRO-3 (labels nucleus) and were taken using Leica TS5 system. Scale bar=20 μm, magnification 63×.

FIG. 7. Illustrates the kinetics of the TfR monoclonal antibody fusion protein as well as the control IgG fusion protein (described in FIG. 6) in both brain and blood of mice following a single intraperitoneal injection. At the designated time following intraperitoneal injection of fusion proteins, serum was first collected and then mouse brain was harvested and perfused with DPBS. The antibody concentration in brain lysate (a) or serum (b) was determined using sandwich ELISA: Briefly, TfR antibody fusion or control antibody fusion was captured on a plate-coated human VEGFA165 (which binds to the fusion of VEGFR), then the amount of antibody present was detected using anti-human Fc-HRP. Sampling points (X axis) indicate days (d) post injection of TfR antibody or the control antibody fusion protein.

FIG. 8. Screening and characterization of anti muTfR mAbs. The process of identifying anti-muTfR Ab4. A total of 400 scFv phage colonies were picked using the Qpix instrument from the 3^{rd} round of panning output; and 38 clones were found to be positive in phage ELISA against muTfR-His. After sequencing, 6 unique scFv clones were converted to full IgG1. BLI assay showed 6 antibodies were able to bind to muTfR-His.

FIG. 9. Characterization of VEGF-Trap/aTfR bispecific antibodies. A. Design of VEGF-Trap and VEGF-Trap/aTfR bispecific antibodies. In the VEGF-Trap/moAb4 design, knob-into-hole mutations were introduced to promote heterodimerization. The TfR Fab was fused to the C-terminus of the CH3. Although not depicted, the human Fc regions contain LALAPG mutations to abolish Fc-mediated immune effector functions. B. Titration curves showing dose-dependent binding of TfR by VEGF-Trap/aTfR bispecific antibodies. VEGF-Trap/biAb4 showed significantly stronger binding to TfR than that of VEGF-Trap/moAb4, n=3 independent repeats. Data points with error bars represent mean±SD.

FIG. 10. Characterization of VEGF-Trap/aTfR bispecific antibody brain entry. A. Illustration showing the design of bispecific antibodies. Antibody concentrations in perfused brains at designated time points after treatment. C. Western blotting showing the level of total TfR in mouse brain lysates 24 hours after treatment. The Western blotting signals were quantified and shown in a bar graph. For all the animal studies, n=5 mice per group. Error bars represent mean±SD. For the statistical analysis, ns=not statistically different, ***P<0.001, two-tailed Student t-test.

FIG. 11. VEGF-Trap/moAb4 bispecific antibody significantly enhanced the anti-angiogenic efficacy of VEGF-Trap. A. Illustration showing the design of bispecific antibodies used in this figure. B. Immunofluorescent staining showing the level of fluorescently labeled albumin in U-87 MG tumors, which serves as an indicator of BBB integrity. Scale bar=20 □m. The immunofluorescence data was quantified and showed in a bar graph, n=3 independent mice. C. Immunofluorescent staining showing the level of CD31 in U-87 MG tumors. Scale bar-20 Dm. The immunofluorescence data was quantified and showed in a bar graph, n=3 independent mice. Bar graphs with error bars represent mean±SD. For the statistical analysis, ns=not statistically different, ***P<0.001, two-tailed Student t-test.

FIG. 12: phage ELISA results showing only the human-cynomolgus TfR cross-reactive clones identified from round 3 panning output. Plate was coated with TfR proteins, then phage particles were incubated with the antigen for 2 hours. Then, anti-M13 HRP was added to detect the bound phage. The phage clone number is used consistently in the entire document to label individual antibodies discovered.

FIG. 13: the phage ELISA OD450 values of unique phage clones (from clones presented in FIG. 1).

FIG. 14: Octet studies to screen 18 purified IgG1 molecules against TfR extra cellular domain (ECD) and Cyno TfR ECD. Antibodies (30 μg/mL) were loaded onto Protein A biosensors, then binding to huTfR or CyTfR is studied using 200 nM antigen concentration. Maximum Octet association signals were plotted.

FIG. 15: Antibody affinity measurement curves for 4G1 against human TfR ECD.

FIG. 16: Antibody affinity measurement curves for 4G1 against Cyno TfR ECD.

FIG. 17: Antibody affinity measurement curves for 5B6 against human TfR ECD.

FIG. 18: Antibody affinity measurement curves for 5B6 against Cyno TfR ECD.

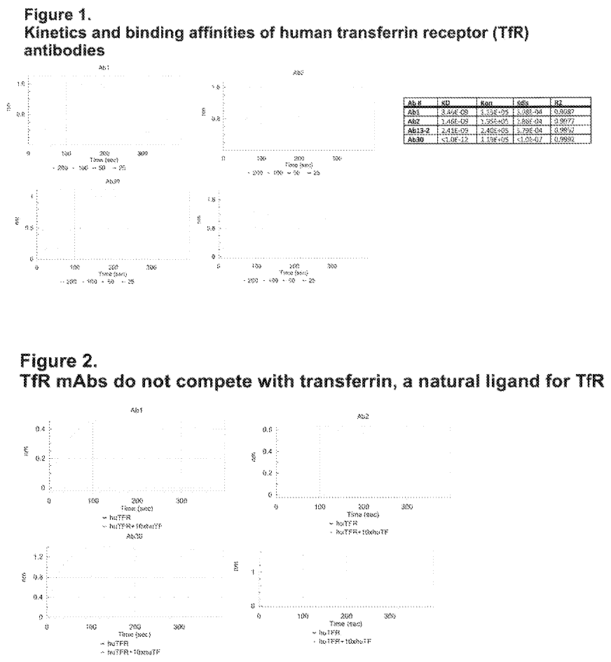

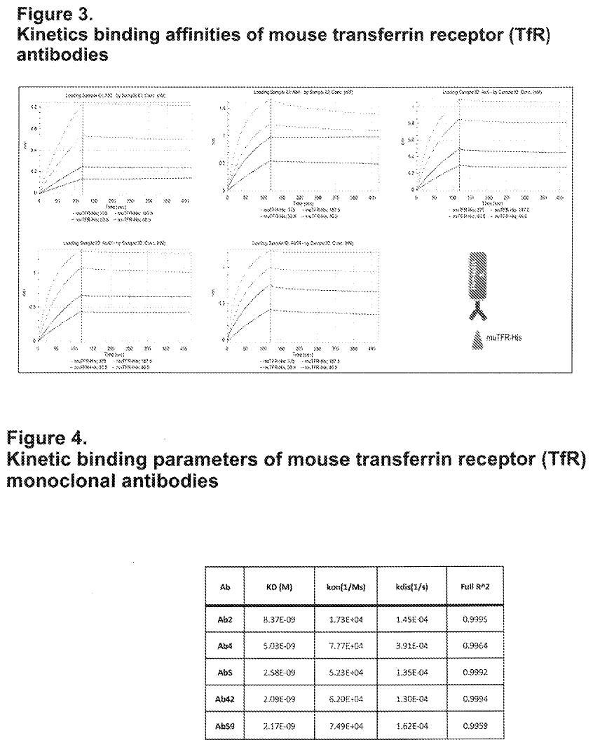

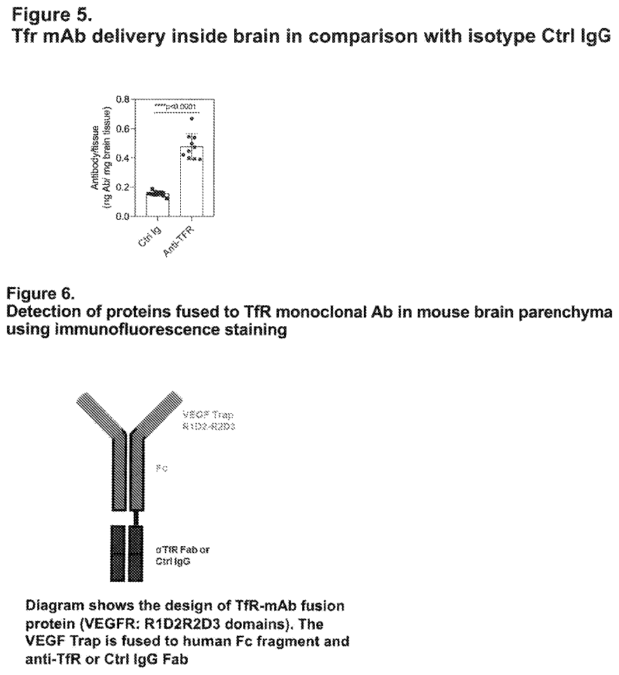

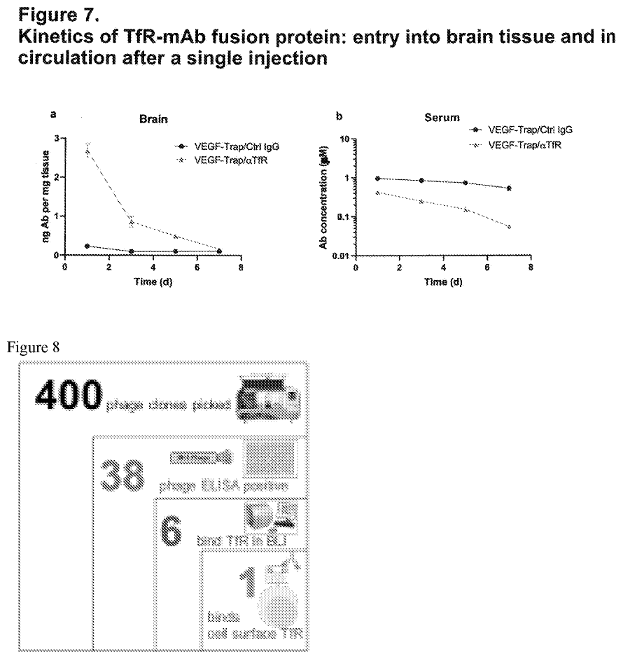

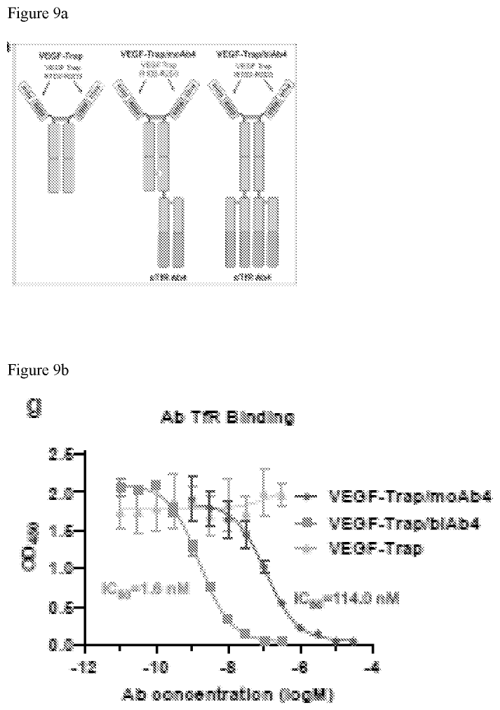

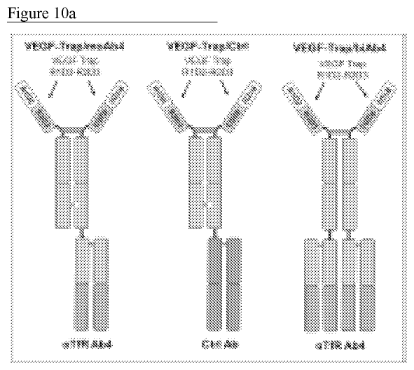

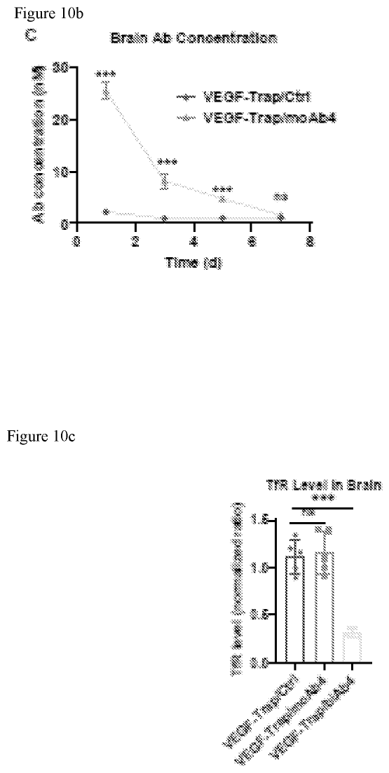

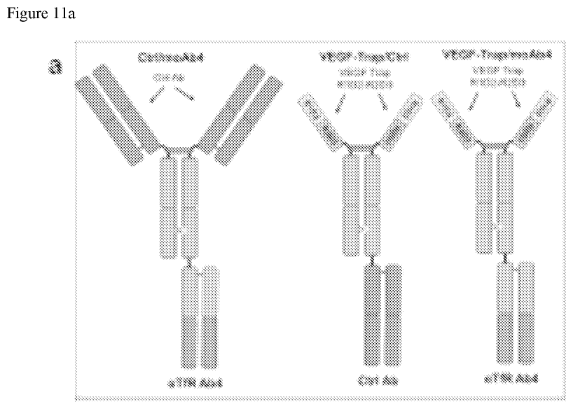

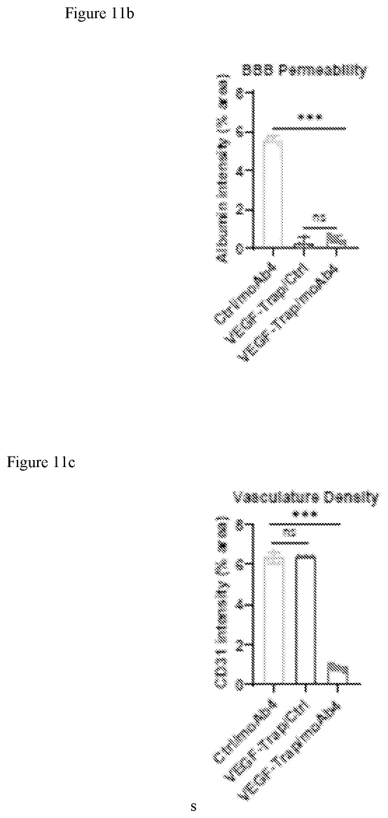

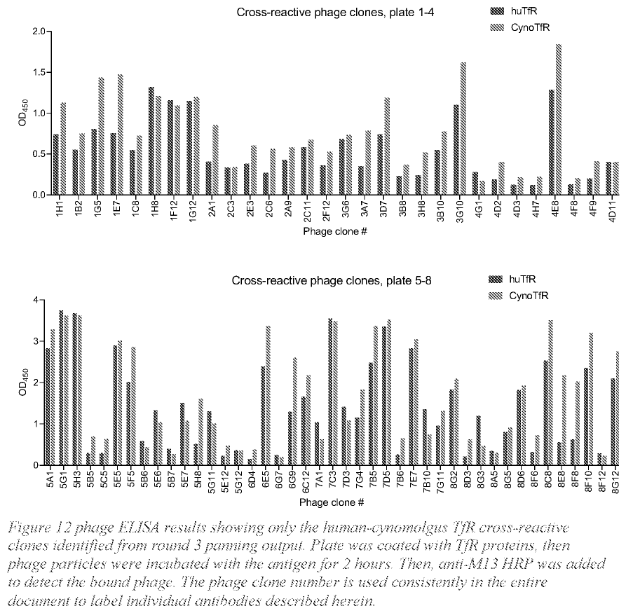

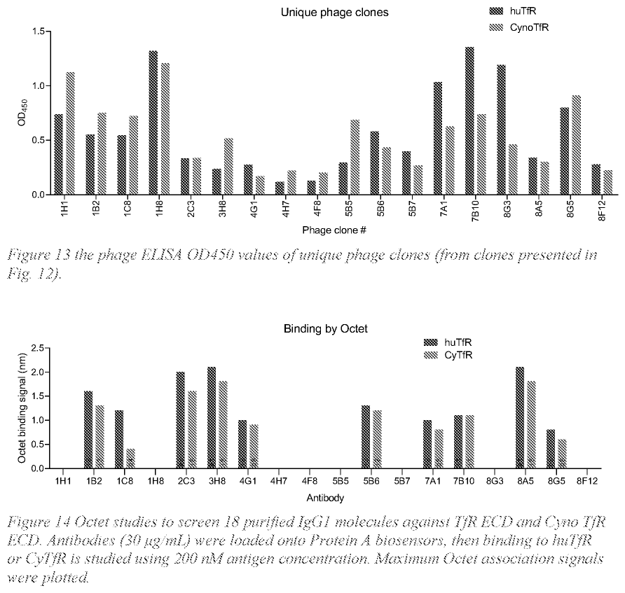

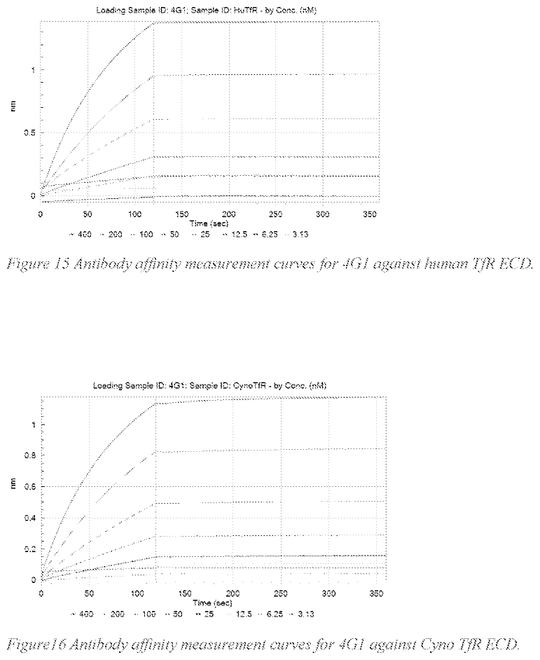

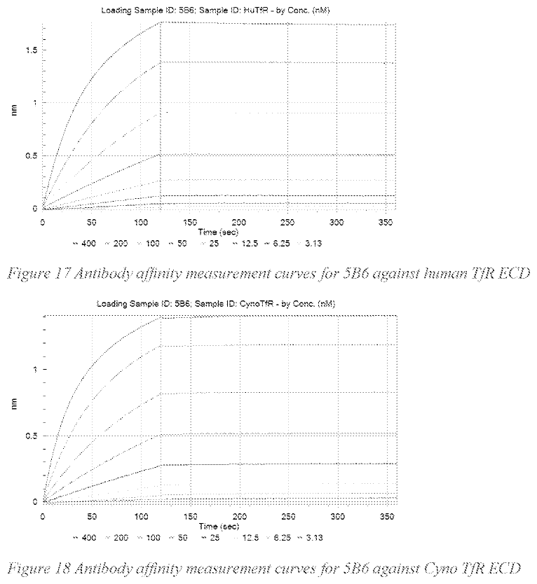

### DETAILED DESCRIPTION

The present disclosure describes a panel of monoclonal antibodies, and fragments thereof, with binding affinity to TfR. The antibodies bind to TfR noncompetitively with transferrin, therefore having no effects on transferrin binding and delivery into cells (N. Zhang, Z. An, and P. Zhao; MAbs. 14:2057269 (2022); DOI: 10.1080/19420862.2022.2057269). The antibodies of the present disclosure can be fused with therapeutic oligopeptides or conjugated with therapeutic small molecules, both of which can be transported (with the anti-TfR antibody) across the blood brain barrier into the brain.

### Antibodies of the Embodiments

In certain embodiments, an antibody or a fragment thereof that binds to at least a portion of TfR protein and facilitates transfer of the antibody or fragment, or conjugates thereof, across the blood-brain barrier. As used herein, the term “antibody” is intended to refer broadly to any immunologic binding agent, such as IgG, IgM, IgA, IgD, IgE, and genetically modified IgG as well as polypeptides comprising antibody CDR domains that retain antigen binding activity. The antibody may be selected from the group consisting of a chimeric antibody, an affinity matured antibody, a polyclonal antibody, a monoclonal antibody, a humanized antibody, a human antibody, or an antigen-binding antibody fragment or a natural or synthetic ligand. Preferably, the TfR-binding antibody is a monoclonal antibody or a humanized antibody.

An “antibody molecule” encompasses an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), for example IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

The term “antigen binding portion” of an antibody molecule, as used herein, refers to one or more fragments of an intact antibody that retain the ability to specifically bind to the target molecule (e.g., TfR). Antigen binding functions of an antibody molecule can be performed by fragments of an intact antibody. Examples of binding fragments encompassed within the term “antigen binding portion” of an antibody molecule include Fab; Fab′; F(ab′)2; an Fd fragment consisting of the V_{H} and CH1 domains; an Fv fragment consisting of the V_{L} and V_{H} domains of a single arm of an antibody; a single domain antibody (dAb) fragment, and an isolated complementarity determining region (CDR).

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. The Fc region of an immunoglobulin generally comprises two constant domains, CH2 and CH3. As is known in the art, an Fc region can be present in dimer or monomeric form.

A “variable region” of an antibody refers to the variable region of the antibody light chain or the variable region of the antibody heavy chain, either alone or in combination. As known in the art, the variable regions of the heavy and light chain each consist of four framework regions (FRs) connected by three complementarity determining regions (CDRs) also known as hypervariable regions, contribute to the formation of the antigen binding site of antibodies. When choosing FR to flank CDRs, for example when humanizing or optimizing an antibody, FRs from antibodies which contain CDR sequences in the same canonical class are preferred.

As used herein the term “conservative substitution” refers to replacement of an amino acid with another amino acid which does not significantly deleteriously change the functional activity. A preferred example of a “conservative substitution” is the replacement of one amino acid with another amino acid which has a value greater than 0 in a BLOSUM 62 substitution matrix (see Henikoff & Henikoff, 1992, PNAS 89: 10915-10919).

Thus, by known means and as described herein, monoclonal antibodies, antibody fragments, and binding domains and CDRs (including engineered forms of any of the foregoing) may be created that are specific to the TfR protein, one or more of its respective epitopes, or conjugates of any of the foregoing, whether such antigens or epitopes are isolated from natural sources or are synthetic derivatives or variants of the natural compounds.

Examples of antibody fragments suitable for the present embodiments include, without limitation: (i) the Fab fragment, consisting of V_{L}, V_{H}, CL, and CHI domains; (ii) the “Fd” fragment consisting of the V_{H} and CHI domains; (iii) the “Fv” fragment consisting of the V_{L} and V_{H} domains of a single antibody; (iv) the “dAb” fragment, which consists of a V_{H} domain; (v) isolated CDR regions; (vi) F(ab′)2 fragments, a bivalent fragment comprising two linked Fab fragments; (vii) single chain Fv molecules (“scFv”), wherein a V_{H} domain and a V_{L} domain are linked by a peptide linker that allows the two domains to associate to form a binding domain; (viii) bi-specific single chain Fv dimers (see, for example, U.S. Pat. No. 5,091,513); and (ix) diabodies, multivalent or multispecific fragments constructed by gene fusion (see, for example, US Patent App. Pub. No. 20050214860, which is incorporated herein by reference in its entirety). Fv, scFv, or diabody molecules may be stabilized by the incorporation of disulphide bridges linking the V_{H} and V_{L} domains. Minibodies comprising a scFv joined to a CH3 domain may also be made (See, for example, Hu et al, 1996, “Minibody: A Novel Engineered Anti-Carcinoembryonic Antigen Antibody Fragment (Single-Chain Fv-CH3) Which Exhibits Rapid, High-Level Targeting of Xenografts”, Cancer Res. 56:3055-3061, which is incorporated herein by reference in its entirety).

Antibody-like binding peptidomimetics are also contemplated in embodiments. Liu et al. (Murali, R.; Liu, Q.; Cheng, X.; Berezov, A.; Richter, M.; Furuchi, K.; Greene, M. L; Zhang, R. Antibody like peptidomimetics as large scale immunodetection probes. Cell. Mol. Biol. (Noisy-le-grand) 2003, 49:209--216, which is incorporated herein by reference in its entirety) describe “antibody like binding peptidomimetics” (ABiPs), which are peptides that act as pared-down antibodies and have certain advantages of longer serum half-life as well as less cumbersome synthesis methods.

A monoclonal antibody (or “MAb”) is a single species of antibody wherein every antibody molecule recognizes the same epitope because all antibody producing cells are derived from a single B-lymphocyte cell line. The methods for generating monoclonal antibodies (MAbs) generally begin along the same lines as those for preparing polyclonal antibodies. In some embodiments, rodents such as mice and rats are used in generating monoclonal antibodies. In some embodiments, rabbit, sheep, or frog cells are used in generating monoclonal antibodies. The use of rats is well known and may provide certain advantages. Mice (e.g., BALB/c mice) are routinely used and generally give a high percentage of stable fusions.

Hybridoma technology involves the fusion of a single B lymphocyte from a mouse previously immunized, for example, with a TfR antigen with an immortal myeloma cell (usually mouse myeloma). This technology provides a method to propagate a single antibody producing cell for an indefinite number of generations, such that unlimited quantities of structurally identical antibodies having the same antigen or epitope specificity (monoclonal antibodies) may be produced.

Plasma B cells (CD45+CD5−CD19+) may be isolated from freshly prepared rabbit peripheral blood mononuclear cells of immunized rabbits and further selected for TfR binding cells. After enrichment of antibody producing B cells, total RNA may be isolated and cDNA synthesized. DNA sequences of antibody variable regions from both heavy chains and light chains may be amplified, constructed into a phage display Fab expression vector, and transformed into E. coli. TfR specific binding Fab may be selected out through multiple rounds enrichment panning and sequenced. Selected TfR binding hits may be expressed as full-length IgG in rabbit and rabbit/human chimeric forms using a mammalian expression vector system in human embryonic kidney (HEK293) cells (Invitrogen) and purified using a protein G resin with a fast protein liquid chromatography (FPLC) separation unit.

In one embodiment, the antibody is a chimeric antibody, for example, an antibody comprising antigen binding sequences from a non-human donor grafted to a heterologous non-human, human, or humanized sequence (e.g., framework and/or constant domain sequences). Methods have been developed to replace light and heavy chain constant domains of the monoclonal antibody with analogous domains of human origin, leaving the variable regions of the foreign antibody intact. Alternatively, “fully human” monoclonal antibodies are produced in mice transgenic for human immunoglobulin genes. Methods have also been developed to convert variable domains of monoclonal antibodies to more human form by recombinantly constructing antibody variable domains having both rodent, for example, mouse, and human amino acid sequences. In “humanized” monoclonal antibodies, only the hypervariable CDR is derived from mouse monoclonal antibodies, and the framework and constant regions are derived from human amino acid sequences (see, for example, U.S. Pat. Nos. 5,091,513 and 6,881,557, which are incorporated herein by reference in their entirety). It is thought that replacing amino acid sequences in the antibody that are characteristic of rodents with amino acid sequences found in the corresponding position of human antibodies will reduce the likelihood of adverse immune reaction during therapeutic use. A hybridoma or other cell producing an antibody may also be subject to genetic mutation or other changes, which may or may not alter the binding specificity of antibodies produced by the hybridoma.

Methods for producing polyclonal antibodies in various animal species, as well as for producing monoclonal antibodies of various types, including humanized, chimeric, and fully human, are well known in the art and highly predictable. For example, the following U.S. patents and patent applications, which are incorporated herein by reference in their entirety, provide enabling descriptions of such methods: U.S. Patent Application Nos. 2004/0126828 and 2002/0172677; and U.S. Pat. Nos. 3,817,837; 3,850,752; 3,939,350; 3,996,345; 4,196,265; 4,275,149; 4,277,437; 4,366,241; 4,469,797; 4,472,509; 4,606,855; 4,703,003; 4,742,159; 4,767,720; 4,816,567; 4,867,973; 4,938,948; 4,946,778; 5,021,236; 5,164,296; 5,196,066; 5,223,409; 5,403,484; 5,420,253; 5,565,332; 5,571,698; 5,627,052; 5,656,434; 5,770,376; 5,789,208; 5,821,337; 5,844,091; 5,858,657; 5,861,155; 5,871,907; 5,969,108; 6,054,297; 6,165,464; 6,365,157; 6,406,867; 6,709,659; 6,709,873; 6,753,407; 6,814,965; 6,849,259; 6,861,572; 6,875,434; and 6,891,024.

Antibodies may be produced from any animal source, including birds and mammals. Preferably, the antibodies are ovine, murine (e.g., mouse and rat), rabbit, goat, guinea pig, camel, horse, or chicken. In addition, newer technology permits the development of and screening for human antibodies from human combinatorial antibody libraries. For example, bacteriophage antibody expression technology allows specific antibodies to be produced in the absence of animal immunization, as described in U.S. Pat. No. 6,946,546, which is incorporated herein by reference.

In some embodiments, the production of polynucleotides encoding TrF-binding monoclonal antibodies comprised using human phage antibody libraries panned with recombinantly-expressed TrF-His select to enrich and isolate high-affinity TrF-binding bacteriophage. The DNA sequence of each bacteriophage clone was determined and the sequences were analyzed using GeneBank IgBLAST to identify germline V(D)J gene segments. Individual VH and VL genes were mapped to the germline of major IGL and IGH locus. CDR sequences were annotated according to IMGT (http://www.imgt.org/) nomenclature.

DNA fragments encoding VH and VL chains were amplified by PCR using gene specific primers. The PCR products of VH and VL gene fragments were gel-extracted and purified to make full length heavy chain (HC) and light chain (LC) DNA constructs using an infusion cloning kit (In-Fusion® HD Cloning kit, Clontech).

Expression of anti-TrF monoclonal antibodies: Human anti-TrF antibodies were produced in mammalian cells (Expi 293 cells from Thermo Fisher) by transiently transfecting HEK293 cells with paired HC and LC containing DNA constructs. Antibodies in the culture medium were purified (isolated) using Protein A resin according to a method based on the manufacturer's (Repligen) instructions.

It is fully expected that antibodies to TfR (and conjugates thereof) will, by binding to TfR, have the ability to cross the blood-brain barrier (BBB), bringing (e.g., bound or conjugated) therapeutic medications therewith. Certain animal species may be less preferable for generating therapeutic antibodies because they may be more likely to cause allergic response due to activation of the complement system through the “Fc” portion of the antibody. However, whole antibodies may be enzymatically digested into “Fc” (complement binding) fragment, and into antibody fragments having the binding domain or CDR. Removal of the Fc portion reduces the likelihood that the antigen antibody fragment will elicit an undesirable immunological response, and thus, antibodies without Fc may be preferential for prophylactic or therapeutic treatments. As described above, antibodies may also be constructed so as to be chimeric or partially or fully human, so as to reduce or eliminate the adverse immunological consequences resulting from administering to an animal an antibody that has been produced in, or has sequences from, other species.

Substitutional variants typically contain the exchange of one amino acid for another at one or more sites within the monoclonal antibody protein and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Proteins (e.g., monoclonal antibodies) of the present disclosure may be isolated (e.g., enriched and/or purified to some degree) and/or may be recombinant or synthesized in vitro. Alternatively, a nonrecombinant or recombinant protein may be isolated from bacteria. It is also contemplated that a bacteria containing such a variant may be implemented in compositions and methods. Consequently, a protein need not be isolated.

Thus, the present disclosure provides an isolated or recombinant monoclonal antibody that specifically binds to TfR. In certain aspects, an antibody that competes for the binding of TfR with 1B2, 1C8, 2C3, 3H8, 4G1, 5B6, 7A1, 7B10, 8A5 or 8G5 monoclonal antibody (each disclosed and described herein) is provided. In certain aspects, the antibody may comprise all or part of the heavy chain variable region and/or light chain variable region of the 1B2, 1C8, 2C3, 3H8, 4G1, 5B6, 7A1, 7B10, 8A5, 8G5, mTfR-2, mTfR-4, mTfR5, mTfR-42, mTfR-59, hTfR-1, hTfR-1, hTfR-1, and hTfR-1 monoclonal antibodies.

It is contemplated that in compositions of the present disclosure there is between about 0.001 mg and about 10 mg of total polypeptide, peptide, and/or protein per ml. Thus, the concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein). Of this, about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% may be an antibody that binds TfR.

An antibody or preferably an immunological portion of an antibody, can be chemically conjugated to, or expressed as, a fusion protein with other proteins. For purposes of this specification and the accompanying claims, all such fused proteins are included in the definition of antibodies or an immunological portion of an antibody.

Embodiments provide antibodies and antibody-like molecules against TrF, polypeptides and peptides that are linked to at least one agent to form an antibody conjugate or payload. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules that have been attached to antibodies include toxins, therapeutic enzymes, antibiotics, radio-labeled nucleotides and the like. By contrast, a reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3a-6a-diphenylglycouril attached to the antibody. Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate.

### Chimeric Antigen Receptors

As used herein the term “chimeric antigen receptor” or “CAR” refers to an artificially constructed hybrid protein or polypeptide containing an antigen-binding domain of an antibody (e.g., a single chain variable fragment (scFv)) linked to a domain or signaling, e.g., T-cell signaling or T-cell activation domains, that activates an immune cell, e.g., a T cell or a NK cell. CARs are capable of redirecting the immune cell specificity and reactivity toward a selected target in a non-MHC-restricted manner, taking advantage of the antigen-binding properties of monoclonal antibodies. This non-MHC-restricted antigen recognition confers on immune cells expressing CARs the ability to recognize an antigen independent of processing, thus bypassing a mechanism of tumor escape. In another aspect, provided is a chimeric antigen receptor (CAR) protein comprising an antigen-binding fragment as provided herein. In another aspect, provided is an isolated nucleic acid that encodes a CAR protein as provided herein.

In another aspect, an engineered cell comprising the isolated nucleic acid as provided herein. In certain embodiments, the engineered cell is a T cell, NK cell, or myeloid cell. In another aspect, the present disclosure provides immune cells which express a chimeric antigen receptor (CAR). In some embodiments, the CAR comprises an antigen-binding fragment provided herein. In some embodiments, the CAR protein includes from the N-terminus to the C-terminus: a leader peptide, an anti-TfR heavy chain variable domain, a linker domain, an anti-TfR light chain variable domain, a human IgG1-CH2-CH3 domain, a spacer region, a CD28 transmembrane domain, an anti-TfR intracellular co-stimulatory signaling and a CD3z intracellular T cell signaling domain.

In certain embodiments, the chimeric antigen receptor comprising an antigen-binding domain at least 80%, 85%, 90%, 91%, 92%, 93%, 94%, 95%, 96%, 97%, 98%, 99% or 100% identical to the antigen-binding domain of any one of the TfR-specific monoclonal antibodies disclosed herein. In certain embodiments, the engineered cell expresses an antigen-binding domain at least 80%, 85%, 90%, 91%, 92%, 93%, 94%, 95%, 96%, 97%, 98%, 99% or 100% identical to the antigen-binding domain of any one of the TfR-specific monoclonal antibodies disclosed herein.

### Treatment of Diseases

Certain aspects of the present embodiments can be used to prevent treat, or diagnose, a disease or disorder (e.g., cancers such as glioblastoma, for example and certain dementias such as Alzheimer's Disease, for example) in a human. TfR activity may be increased or reduced by any TfR-binding antibodies. Preferably, such antibodies would be an anti-TfR antibody.

“Treatment” and “treating” refer to administration or application of a therapeutic agent to a subject or performance of a procedure or modality on a subject for the purpose of obtaining a therapeutic benefit of a disease or health-related condition. For example, a treatment may include administration of a pharmaceutically effective amount of an antibody that modulates TfR biological activity.

Treatment may be accomplished by perfusion, direct injection, or local application of the therapeutic agent to the affected area. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

“Subject” and “patient” refer to either a human or non-human, such as primates, mammals, and vertebrates. In particular embodiments, the subject is a human.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease.

### Pharmaceutical Preparations

Where clinical application of a therapeutic composition containing an antibody is undertaken, it will generally be beneficial to prepare a pharmaceutical or therapeutic composition appropriate for the intended application. In certain embodiments, pharmaceutical compositions may comprise, for example, at least about 0.1% of an active compound. In other embodiments, an active compound may comprise between about 2% to about 75% of the weight of the unit, or between about 25% to about 60%, for example, and any range derivable therein.

The therapeutic compositions of the present embodiments are advantageously administered in the form of injectable compositions either as liquid solutions or suspensions; solid forms suitable for solution in, or suspension in, liquid prior to injection may also be prepared. These preparations also may be emulsified.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “unit dose” or “dosage” refers to physically discrete units suitable for use in a subject, each unit containing a predetermined quantity of the therapeutic composition calculated to produce the desired responses discussed above in association with its administration, i.e., the appropriate route and treatment regimen. The quantity to be administered, both according to number of treatments and unit dose, depends on the effect desired. The actual dosage amount of a composition of the present embodiments administered to a patient or subject can be determined by physical and physiological factors, such as body weight, the age, health, and sex of the subject, the type of disease being treated, the extent of disease penetration, previous or concurrent therapeutic interventions, idiopathy of the patient, the route of administration, and the potency, stability, and toxicity of the particular therapeutic substance. For example, a dose may also comprise from about 1 mg/kg/body weight to about 1000 mg/kg/body weight (this such range includes intervening doses) or more per administration, and any range derivable therein. In non-limiting examples of a derivable range from the numbers listed herein, a range of about 5 mg/kg/body weight to about 100 mg/kg/body weight, about 5 mg/kg/body weight to about 500 mg/kg/body weight, etc., can be administered. The practitioner responsible for administration will, in any event, determine the concentration of active ingredient(s) in a composition and appropriate dose(s) for the individual subject.

The active compounds can be formulated for parenteral administration, e.g., formulated for injection via the intravenous, intramuscular, sub-cutaneous, or even intraperitoneal routes. Typically, such compositions can be prepared as either liquid solutions or suspensions; solid forms suitable for use to prepare solutions or suspensions upon the addition of a liquid prior to injection can also be prepared; and the preparations can also be emulsified.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form preferably should be sterile and preferably should be fluid to the extent that it may be easily injected. It also should be stable under the conditions of manufacture and storage and preferably should be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

### Kits and Diagnostics

In various aspects of the embodiments, a kit is envisioned containing therapeutic agents and/or other therapeutic and delivery agents. In some embodiments, the present embodiments contemplate a kit for preparing and/or administering a therapy of the embodiments. The kit may comprise one or more sealed vials containing any of the pharmaceutical compositions of the present embodiments. The kit may include, for example, at least one anti-TfR antibody as well as reagents to prepare, formulate, and/or administer the components of the embodiments or perform one or more steps of the inventive methods. In some embodiments, the kit may also comprise a suitable container, which is a container that will not react with components of the kit, such as an Eppendorf tube, an assay plate, a syringe, a bottle, or a tube. The container may be made from sterilizable materials such as plastic or glass.

The kit may further include an instruction sheet that outlines the procedural steps of the methods set forth herein, and will follow substantially the same procedures as described herein or are known to those of ordinary skill in the art. The instruction information may be in a computer readable media containing machine-readable instructions that, when executed using a computer, cause the display of a real or virtual procedure of delivering a pharmaceutically effective amount of a therapeutic agent.

### EXAMPLES

Unless noted otherwise, data generated from the experiments and Examples described hereinbelow can be found in “Enhanced anti-angiogenetic effect of transferrin receptor-mediated delivery of VEGF-trap in a glioblastoma mouse model”, MABS, 2022, VOL. 14, NO. 1, e2057269 (12 pages); which is incorporated herein by reference in its entirety.)

### Experimental Results

### Generation of TfR Antibodies

To generate antibodies that bind muTfR, a screening strategy from a phage-displayed scFv human antibody library was utilized (Zhao, S., et al., Partial Leptin Reduction as an Insulin Sensitization and Weight Loss Strategy. Cell Metab, 2019. 30(4): p. 706-719.e6). Briefly, 38 scFv clones that bind to the muTfR extracellular domain (ECD) were enriched by phage panning and phage ELISA and were identified from a total of 400 phage clones picked from the 3^{rd} round of panning. The 38 muTfR binding scFv clones were then converted into human IgG1 for binding confirmation by the bio-layer interferometry (BLI) assay. 6 IgG1 antibodies were confirmed that bind to muTfR in the BLI assay. Surprisingly, when the 6 IgG1 antibodies were screened for binding the muTfR expressed on cell surface of 293T cells, only 1 of 6 antibodies (Ab4) bound cell surface expressed TfR.

Charactering the Competition of Ab4 with Transferrin (Tf, which is the Natural Ligand of TfR.

Tf binds to TfR at high affinity at neutral pH (Giannetti, A. M., et al., Mechanism for multiple ligand recognition by the human transferrin receptor. PLoS Biol, 2003. 1(3): p. E51). Tf presents in serum at a very high concentration of about 3 mg/mL (Wessling-Resnick, M., Crossing the Iron Gate: Why and How Transferrin Receptors Mediate Viral Entry. Annual review of nutrition, 2018. 38: p. 431-458). Therefore, any antibody that competes with Tf will not be able to bind to TfR, rendering the antibody ineffective. Moreover, competing with Tf binding to TfR may impede the normal iron delivery into cells. HEK293T cells overexpressing muTfR were co-incubated the Ab4 with an excessively high concentration of muTf (10 mM) and detected no differences in antibody binding to cell surface muTfR, suggesting that Ab4 binds to muTfR specifically on cell surface without being blocked by Tf. While not wishing to be bound by any specific mechanism, Ab4 may bind to the apical domain of TfR. Based on the construction of a chimeric huTfR receptor with its apical domain replaced by the corresponding apical domain from muTfR (huTfR-muTfR apical domain); and observed that the muTfR apical domain alone is sufficient to enable Ab4 binding to the chimeric huTfR-muTfR apical domain receptor. Ab4 does not cross-react with the human TfR, although the percent of identity between the apical domains of the mouse and human TfRs is 70%.

### VEGF-Trap Bispecific Antibody Characterization

Two bispecific antibodies were created by incorporating VEGF-Trap and muTfR Ab4. The VEGF-Trap was designed based on aflibercept, which is the fusion protein of the D2 domain of VEGFR1, D3 domain of VEGFR2, and human Fc fragment (Holash, J., et al., VEGF-Trap: a VEGF blocker with potent antitumor effects. Proc Natl Acad Sci USA, 2002. 99(17): p. 11393-8). Ab4 was fused to the C terminus of the VEGF-Trap in Fab format. TfR targeting antibodies with full Fc effector functions have been shown to deplete reticulocytes and cause acute toxicities (Couch, J. A., et al., Addressing safety liabilities of TfR bispecific antibodies that cross the blood-brain barrier. Sci Transl Med, 2013. 5(183): p. 183ra57, 1-12). To avoid Fc-mediated effector functions, LALAPG mutations (L234A, L235A, and P329G) were introduced to abolish interactions with Fc receptors in humans and in mice (Wang, X., M. Mathieu, and R. J. Brezski, IgG Fc engineering to modulate antibody effector functions. Protein & cell, 2018. 9(1): p. 63-73; Schlothauer, T., et al., Novel human IgG1 and IgG4 Fc-engineered antibodies with completely abolished immune effector functions. Protein Eng Des Sel, 2016. 29(10): p. 457-466). For the monovalent TfR bispecific design (VEGF-Trap/moAb4), the KiH mutations (knob: T366W and S354C; hole: T366S, L368A, Y407V, and Y349C) were incorporated to promote heterodimerization between the heavy chains (Schaefer, W., et al., Immunoglobulin domain crossover as a generic approach for the production of bispecific IgG antibodies. Proc Natl Acad Sci USA, 2011. 108(27): p. 11187-92). The TfR Ab fusion arm was introduced with the “hole” mutations, while the other arm bears the “knob” mutations. For the bivalent TfR bispecific design (VEGF-Trap/biAb4), a homodimer of VEGF-Trap fusion with Ab4 from the N-terminus to the C-terminus was used, resulting a bivalency for both VEGF-Trap and Ab4.

To characterize the bispecific antibodies a BLI-based sandwich capture assay. The bispecific antibodies were first captured onto sensors via VEGF165A. After equilibrium in blank buffer, the sensor-captured bispecific antibodies were then incubated with muTfR ECD. The VEGF-Trap/TfR bispecific antibodies were shown simultaneously binding to both VEGF165 and TfR. To further validate the antigen binding, we introduced another three control groups that missed one of the three binding partners (VEGFA, Ab, or TfR). Omitting VEGFA showed a complete flat curve, which confirmed the observed binding signals in curves A and E are dependent on the proteins being captured by VEGFA, Curves B and F). Omitting Ab showed a flat curve when the sensors were dipped into TfR solution, which confirmed the observed TfR binding signals in curves A and E are dependent on the existence of antibodies. Finally, the flat curves without TfR confirmed the observed binding signals in curves A and E were antibodies binding to TfR. Taken together, these data showed that both the bivalent and monovalent TfR bispecific antibodies VEGF-Trap/moAb4 and VEGF-Trap/biAb4 can simultaneously engage both VEGF and TfR.

Ab4 does not Interfere with TfR from Binding to its Ligand Tf, Suggesting that the Antibody Will not Interfere with the Natural Functions of TfR.

To demonstrate that the bispecific antibodies can trigger cellular endocytosis, which facilitates effective transcytosis of the antibodies through TfR. Using the mouse endothelial BEnd.3 cells, we showed concentration-dependent endocytosis of the bispecific antibodies bearing either bivalent or monovalent TfR Ab4. As a negative control, endocytosis was abolished when the cells and antibodies were incubated at 4° C. Of note, bivalent and monovalent TfR Ab-bearing bispecific antibodies showed similar levels of endocytosis across all concentrations.

### Antibody-Mediated TfR Endocytosis has an Impact on the Level of TfR Cell Surface Expression.

Naturally, TfR endocytosis delivers transferrin into endosomes, where the Tf releases iron, and the TfR-Tf complex recycles back to the cell surface (Wessling-Resnick, M., Crossing the Iron Gate: Why and How Transferrin Receptors Mediate Viral Entry. Annual review of nutrition, 2018. 38: p. 431-458). We measured TfR cell surface levels after incubation with bispecific antibodies. Similar to the VEGF-Trap negative control, monovalent TfR Ab-bearing bispecific antibody VEGF-Trap/moAb4 showed no reduction of cell surface TfR levels. In contrast, the bivalent bispecific antibody (VEGF-Trap/biAb4)-treated BEnd.3 cells demonstrated concentration-dependent reduction of cell surface TfR levels, and at 100 nM, the surface TfR was reduced to undetectable levels. As a control, co-incubation with the lysosomal inhibitor Baf significantly decreased or eliminated the reduction of surface TfR levels by the bivalent bispecific antibody VEGF-Trap/biAb4. While not wishing to be bound by any specific mechanism the data suggests that the bivalent TfR antibody VEGF-Trap/biAb4 induces the decrease of cell surface TfR level by promoting its lysosomal degradation. The possibility that the bivalent antibody induced TfR re-localization intracellularly was eliminated by treating BEnd.3 cells with the two bispecific antibodies and measuring total TfR protein levels by Western blotting. The total TfR levels in BEnd.3 cells are similar between VEGF-Trap-treated and VEGF-Trap/moAb4-treated groups. In contrast, the VEGF-Trap/biAb4 treatment significantly diminished the total TfR protein levels. As a control, co-incubation of lysosomal inhibitor Baf was able to prevent the decrease of total TfR levels mediated by VEGF-Trap/biAb4 treatment, confirming that the bivalent antibody induces TfR lysosomal degradation.

Human endothelial cell line HUVEC has been the gold standard to evaluate the efficacy of anti-angiogenesis therapeutics (Holash, J., et al., VEGF-Trap: a VEGF blocker with potent antitumor effects. Proc Natl Acad Sci USA, 2002. 99(17): p. 11393-8; Robinson, C. J., et al., The World Health Organization reference reagent for vascular endothelial growth factor, VEGF165. Growth Factors, 2006. 24(4): p. 285-90) and the inhibition of VEGFA-stimulated HUVEC cell proliferation by the two bispecific antibodies under the conditions of depleted growth factors and cytokines. VEGF-Trap/biAb4 and VEGF-Trap/moAb4 bispecific antibodies showed similar dose-dependent inhibition of VEGFA-mediated HUVEC proliferation as the VEGF-Trap positive control.

### Binding Affinity of the TfR Bispecific Antibodies to TfR by Competition ELISA.

ELISA plate was coated with muTfR ECD and a series of concentrations of TfR bispecific antibodies were added in the presence of 1 nM of biotinylated Ab4. The 1 nM Ab4 concentration was predetermined to be sensitive enough in quantifying the amount of unbound TfR, yet the concentration was low enough to not interfere the binding of bispecific TfR antibodies. Both the VEGF-Trap/moAb4 and VEGF-Trap/biAb4 bispecific antibodies showed dose-dependent binding to muTfR, as indicated by the decreased OD₄₅₀ signals from the biotinylated Ab4. In contrast, VEGF-Trap/biAb4 showed a significantly stronger (about 100-fold) occupation (binding) of TfR as compared to the VEGF-Trap/moAb4. Without being bound by theory, this suggests that avidity may play a significant role in the TfR binding for the bivalent bispecific antibody.

### Biodistribution of VEGF-Trap TfR Bispecific Antibodies in the Brain and Serum of Mice

A day after a single intraperitoneal (IP) injection at 20 mg/kg of the antibodies, serum and the brains of the mice were collected. Twenty (20) mg/kg was chosen as the therapeutic dose according to previous publications. The brains were collected following thorough PBS perfusion to avoid the interference from residue blood in the vasculature. A sandwich ELISA was used to quantify the concentration of bispecific antibodies inside the brain. In the sandwich ELISA, the bispecific antibody was first captured by plate coated VEGFA, and then the captured antibody was detected by a secondary antibody. The VEGF-Trap/moAb4 bispecific antibody was present at a significantly higher concentration in the brain than were the VEGF-Trap/biAb4 and the VEGF-Trap. The VEGF-Trap/moAb4 showed a 10-fold increase in brain concentration over the VEGF-Trap/Ctrl and a 5-fold increase over the VEGF-Trap/biAb4. Even though the VEGF-Trap/biAb4 induces significant TfR lysosomal degradation, it still showed a 2-fold increase in brain concentration over the VEGF-Trap/Ctrl. A time course tracking showed that the concentrations of VEGF-Trap/moAb4 in the brain decreased as time elapse, and 24 hours after injection was observed as the highest concentration in the brain. During the same time course, the VEGF-Trap/Ctrl maintained a constantly low level of less than 1 nM.

When serum concentrations of the bispecific antibodies were quantified by the same sandwich ELISA. Serum concentrations of VEGF-Trap/moAb4 and VEGF-Trap/biAb4 bispecific antibodies were significantly lower than that of the VEGF-Trap/Ctrl. The VEGF-Trap/biAb4 concentration was 60% of the VEGF-Trap/moAb4 in the sera. Serum antibody concentrations over the course of a week provide a more complete PK profile. The VEGF-Trap/Ctrl experienced only a 30% decrease over the week, but in comparison, the VEGF-Trap/moAb4 bispecific antibody experienced a significantly more rapid clearance over the same time period.

### Location of the Antibodies in the Brain by Immunofluorescence Staining.

As shown in, the VEGF-Trap/moAb4 showed prominent brain parenchyma distribution. CD31 was co-stained to discern blood vessels and the antibody is trapped inside the blood vessel. In contrast, the VEGF-Trap/biAb4 showed localization within the blood vessel and therefore was likely trapped in the blood vessel without entering the brain parenchyma. Of note, the VEGF-Trap/Ctrl showed almost no distribution in either the blood vessel or the brain parenchyma.

Since the VEGF-Trap/biAb4 induced quick degradation of TfR in vitro, it was tested in vivo. The VEGF-Trap/biAb4 induced a significant decrease in the TfR concentration in the brain as measured by Western blotting. In comparison, VEGF-Trap/Ctrl and the VEGF-Trap/moAb4 induced no changes in the amount of brain TfR. Without being bound by theory, these collective data suggest that the VEGF-Trap/biAb4 bispecific antibody induces degradation of TfR in vivo.

### Inhibition of Tumor Angiogenesis

To determine if delivering VEGF-Trap via TfR bispecific antibody can improve the anti-angiogenesis effect of VEGF-Trap by overcoming the BBB blockade in the U-87 MG model, a human GBM model with known BBB leakage (Brighi, C., et al., Comparative study of preclinical mouse models of high-grade glioma for nanomedicine research: the importance of reproducing blood-brain barrier heterogeneity. Theranostics, 2020. 10(14): p. 6361-6371).

Three antibodies were used in the U-87 MG GBM studies. The change of BBB permeability after VEGF-Trap treatment was first validated. To assess BBB permeability, animals were injected with fluorescently labeled albumin molecules 2 hours before sacrifice. Ctrl/moAb4-treated mice showed significant BBB leakage as indicated by the albumin signals in the tumor. In comparison, the VEGF-Trap/Ctrl treatment resulted in significantly lower albumin signals in the tumor, which indicates low BBB permeability. Similar to VEGF-Trap/Ctrl, the amount of detected albumin in VEGF-Trap/moAb4-treated tumors were also low. Without being bound by theory, these collective data suggest that VEGF-Trap treatment may restore the integrity of the BBB with low permeability to macromolecules. The TfR-targeted VEGF-Trap/moAb4 bispecific antibody enhanced the inhibition of angiogenesis by the increased brain access of VEGF-Trap. The angiogenesis in the tumor was characterized by immunostaining of the endothelial cell marker CD31 (Scholz, A., et al., Endothelial cell-derived angiopoietin-2 is a therapeutic target in treatment-naive and bevacizumab-resistant glioblastoma. EMBO Mol Med, 2016. 8(1): p. 39-57). VEGF-Trap/moAb4 bispecific antibody treatment showed significantly reduced CD31 intensity in the tumor in comparison to Ctrl/moAb4 while the control antibody construct VEGF-Trap/Ctrl, demonstrated no difference of CD31 intensity between Ctrl/moAb4 and VEGF-Trap/Ctrl was observed. Without being bound by theory, these collective data suggest enhanced VEGF-Trap/moAb4 bispecific brain entry was translated into improved anti-angiogenesis efficacy.

### Examples: Materials and Methods Utilized

### Cell Lines

HEK293T, U-87 MG, and BEnd.3 cell lines were acquired from the American Type Culture Collection (ATCC) and cultured in DMEM+10% FBS. HUVEC was also acquired from the ATCC and maintained in F-12K medium supplemented with 0.1 mg/mL heparin, 10% FBS, 30 μg/mL Endothelial Cell Growth Supplement (ECGS).

### Panning of Phage-Displayed Antibody Library

A phage-displayed scFv antibody library was prepared previously (Zhao, S., et al., Partial Leptin Reduction as an Insulin Sensitization and Weight Loss Strategy. Cell Metab, 2019. 30(4): p. 706-719.e6). Panning of the library for muTfR specific antibodies was carried out as described previously with modifications (Zhao, S., et al., Partial Leptin Reduction as an Insulin Sensitization and Weight Loss Strategy. Cell Metab, 2019. 30(4): p. 706-719.e6). Briefly, MaxiSorp Nunc-Immuno tubes (Thermo Fisher Scientific) were coated with 20 □g/mL muTfR-His in DPBS overnight at 4° C. Unbound antigen was removed after washing with DPBS. After blocking the surface with 5% milk in DPBS, the phage library was incubated with the coated-muTfR for 2 hours at room temperature in 5% milk. After washing with PBS+0.05% tween-20 to remove unbound phage, captured phage was eluted by incubating with 100 mM TEA for 20 min. Eluted phage-infected log-phase growing E. coli TG1 were amplified on 2× YTAG agar 500 cm² square plate (Corning) at 30° C. overnight. The amplified phage infected TG1 was used to prepare the phage for the next round of panning using the M13KO7 helper phage. The enrichment process was done in three rounds using the output from the previous round as the input for the next round.

After three rounds of panning, the output titer was measured and single colonies were used to prepare phage for ELISA. High-binding ELISA plates (Corning) were coated with muTfR-His at 2 □g/mL overnight at 4° C. After blocking with 5% milk in PBS, phage prepared from single TG1 colonies in 5% milk PBS was incubated with coated muTfR for 1 hour at room temperature. After washing with PBS+0.05% Tween-20, anti-M13-HRP (Santa Cruz Biotechnology) was added at 1:2000 concentration and incubated for 1 hour at room temperature. After washing with PBS+0.05% Tween-20, TMB substrate (Thermo Fisher Scientific) was added and incubated for 5 min before stopping by 1N H₂SO₄. OD values were read at 450 nm. Top 20% high-binding clones were selected. Phagemids were extracted using Qiagen BioRobot Universal System in 96-well format. After DNA sequencing, sequences were analyzed using the IMGT V-quest service to identify antibody sequences with unique CDR3 regions.

Conversion of Phage scFv to IgG

Unique scFv clones were converted into human IgG1 using mixed universal primers with degeneracy as reported previously (Zhao, S., et al., Partial Leptin Reduction as an Insulin Sensitization and Weight Loss Strategy. Cell Metab, 2019. 30(4): p. 706-719.e6). Individual heavy and light variable chains were amplified using PrimeStar GXL polymerase (Takara Bio). Gel-purified variable chain fragments were cloned into digested vectors using In-Fusion HD cloning enzyme mix (Takara Bio). After the converted plasmid was sequenced, sequences of verified IgG plasmids were transfected into Expi293 cells at the 2-mL scale. After culturing for 5 days, cells were removed and antibody-containing supernatant was collected for screening assay.

For milligram-scale antibody purification, Expi293-produced antibodies were purified using CaptivA Protein A affinity resin (Repligen) and eluted with 0.1M glycine (pH=2.5) and then neutralized with 1/20 volume 1M Tris-HCl (pH=9). Buffer exchange to DPBS was done using Amicon Ultra-15 ultrafiltration units (Mw cutoff=30 k) (MilliporeSigma).

### TfR-Expressing 293T Generation

HEK293T expressing full-length mouse and human TfR or the chimeric receptor were generated using lentivirus. Briefly, the receptor genes were cloned into the pCDH-CMV-MCS-EF1α-Puro vector downstream of the CMV promoter. The 293T cell lines were generated by transducing with packaged lentivirus (generated using the transfer plasmid, pCMV-VSV-G (Addgene 8454), pCMV delta R8.2 (Addgene 12263)). Cells expressing the transgene were selected by 1 μg/mL puromycin until a sufficient number of cells with transgene emerged.

### Bispecific Antibody Validation by BLI

Streptavidin sensors (Fortebio) were used to capture biotinylated VEGFA proteins (Sino Biological). During all incubation steps, samples were kept at room temperature with 1000 rpm shaking. In the VEGFA loading step, 100 nM biotinylated VEGFA proteins were incubated with the sensors for the designated time. In the bispecific antibody interaction steps, 200 nM antibodies were used. In the muTfR incubation step, 100 nM muTfR-His (Sino Biological) were used. Between incubations, the sensors were dipped into blank kinetic buffers to allow the free dissociation of proteins.

### Antibody Endocytosis

A total of 5×10⁴ BEnd.3 cells were incubated with antibodies at designated concentrations and temperature for 2 hours. The antibodies were pre-labeled with Alexa Fluor 488 NHS (ThermoFisher). After incubation, unbound antibodies were removed by centrifugation at 500 g for 5 min. Trypan blue solution (0.2%) was incubated with cells for 5 min to quench the cell surface-bound antibody fluorescence. Cells were then transferred into a V-bottom 96-well plate and washed twice by 350 g 5 min centrifugation. The endocytosis was quantified using the iQue3 high throughput flow cytometer (Sartorius) with at least 10,000 live cells collected.

### Immunoblotting

Cell lysate or brain lysates were obtained by lysing cells or brain tissues using NP-40 lysis buffer (1% NP40, 50 mM Tris-HCl, pH=8, 150 mM NaCl) with Halt™ Protease and Phosphatase Inhibitor Cocktail (100×) (ThermoFisher) for 1 hour with shaking. After removing debris by centrifugation, the total protein amount was normalized by Pierce BCA Protein Assay Kit (ThermoFisher). Protein samples were resolved by 10% SDS-polyacrylamide gels (BIO-RAD) and later transferred onto Immun-Blot PVDF membranes (BIO-RAD). Proteins were probed with specific primary antibodies and secondary antibodies diluted in 5% BSA TBST (Zhong, L., et al., Amyloid-beta modulates microglial responses by binding to the triggering receptor expressed on myeloid cells 2 (TREM2). Mol Neurodegener, 2018. 13(1): p. 15; Zhao, Y., et al., TREM2 Is a Receptor for P-Amyloid that Mediates Microglial Function. Neuron, 2018. 97(5): p. 1023-1031.e7; Chen, H.-M., et al., Blocking immunoinhibitory receptor LILRB2 reprograms tumor-associated myeloid cells and promotes antitumor immunity. The Journal of Clinical Investigation, 2018. 128(12): p. 5647-5662). Antibodies used were TfR (Santa Cruz, 1:1000) and actin-beta (Santa Cruz, 1:1000). The immunoreactive bands were visualized with the West Pico PLUS Chemiluminescent Substrate (ThermoFisher). The immunoreactive bands were quantified using ImageJ. Three independent treatment replicates were conducted with the representative immunoblot shown.

### HUVEC Cell Growth Assay

HUVEC cells maintained in the full growth medium were seeded 1 d before the assay into 96-well plates at the density of 1×10⁴ per well in assay medium (F12K+2% FBS) with 50 ng/mL human VEGFA (Sino Biological) but without the growth factor supplement. To start the assay, the medium was replaced with assay medium with designated antibodies and cultured for another 2 days. The cell viability was quantified using MTS assay (Promega) according to the manufacturer's protocol.

### TfR Occupation Assay

High-binding ELISA plates (Corning) were coated with muTfR-His at 2 μg/mL overnight at 4° C. After blocking with 1% BSA PBS, individual antibodies (at designated concentrations) and 1 nM biotinylated TfR Ab4 in 1% BSA PBS were incubated with coated muTfR for 2 hours at room temperature. After washing with PBS+0.05% Tween-20, streptavidin-HRP (Jackson ImmunoResearch) was added at 1:5000 concentration and incubated for 1 hour at room temperature. After washing with PBS+0.05% Tween-20, TMB substrate (Thermo Fisher Scientific) was added and incubated for 5 min before being stopped by 1N H₂SO₄. OD values were read at 450 nm.

### Antibody Brain Distribution Study

The animal experiments were conducted according to the institutional guidelines with approved protocol AWC-19-0051. BALB/C mice (female, 8-week-old, Jackson Laboratory) were randomly grouped into 5 mice per group. Mice received intraperitoneal injection of antibodies (20 mg/kg) in 0.1 mL DPBS. At the designated time points, blood was collected via tail vein and mice then received transcardial perfusion at 2 mL/min by DPBS for 10 min. Brains were collected with half flash-frozen in liquid nitrogen and another half prepared for cryo-sectioning. For immunofluorescence, the half mouse brains were dipped into 4% PFA for 1 d, then 30% sucrose for 2 d before being embedded into OCT medium (Sakura) and sectioned using Leica Cryostat CM1950 into 40 □m floating sections. The floating sections were stored at 4° C. in PBS with 0.01% sodium azide until use.

### Measurement of Antibody Concentration in Brain and Serum

High-binding ELISA plates (Corning) were coated with human VEGFA (Sino Biological) at 2 μg/mL overnight at 4° C. After blocking with 1% BSA PBS, individual brain lysates were incubated with coated VEGFA for 2 hours at room temperature. After washing with PBS+0.05% Tween-20, anti-human Fc-HRP (Jackson ImmunoResearch) was added at 1:5000 concentration and incubated for 1 hour at room temperature. After washing with PBS+0.05% Tween-20, TMB substrate (Thermo Fisher Scientific) was added and incubated for 5 min before being stopped by 1N H₂SO₄. OD values were read at 450 nm. Standard curves were established using purified corresponding bispecific antibodies following the same method described above.

### Immunofluorescence Staining of Mouse Brains

Floating sections were first blocked in 1% BSA PBS with 0.3% Triton X-100 for 2 hours, then stained with corresponding antibodies: CD31 (1:500, R&D system), human Fc (1:1000, Jackson Immunoresearch), or streptavidin-Alexa Fluor 488 (1:500, Jackson Immunoresearch) in 1% BSA PBS with 0.3% Triton X-100 for overnight at 4° C. with gentle rocking. After washing in PBS 0.3% Triton X-100, corresponding secondary antibodies with fluorescent labeling were incubated with brain slices for 2 hours at 4° C. with gentle rocking. The nucleus was stained with TO-PRO-3 (2 μM) in DPBS for 30 min. Brains slices were imaged using a Leica confocal microscope.

### U-87 MG Xenograft Model

NSG mice (female, 8-week-old, Jackson Laboratory) were randomly grouped into 3 mice per group. The mice were implanted with 5×10⁵ U-87 MG cells in the caudate nucleus using a stereotaxic injection frame. Five days after tumor implantation, mice received an intraperitoneal injection with designated antibodies at 20 mg/kg in 0.2 mL sterile PBS. Four days after injection, all mice were sacrificed and the brains were preserved and cryo-sectioned as described above. For observing mouse survival, the body weight was recorded daily. Any mouse that reaches a 20% bodyweight decrease is considered reaching the experiment endpoint and was euthanized.

### Protein Sequence Analysis

Protein sequence alignment was performed using the T-Coffee multiple sequence alignment server and the alignment figures were generated in ESPript—http://espript.ibcp.fr. (Robert, X. and P. Gouet, Deciphering key features in protein structures with the new ENDscript server. Nucleic Acids Res, 2014. 42(Web Server issue): p. W320-4) The crystal structure was visualized using DeepView-Swiss-PdbViewer, ver 4.1.

### Statistical Analysis

GraphPad Prism (v8, GraphPad Software) was used to generate plots and perform statistical analysis. Statistical differences were determined to be significant at p<0.05 using a two-tailed Student t-test. Data are presented as mean±SD.

The embodiments described above and illustrated in the figures are presented by way of example only and are not intended as a limitation upon the concepts and principles of the present disclosure. As such, it will be appreciated by one having ordinary skill in the art that various changes in the elements and their configuration and arrangement are possible without departing from the spirit and scope of the present disclosure. Various features and aspects of the present disclosure are set forth in the following embodiments.

### Example 1: Identification of Human and Cynomolgus Tfr Cross-Reactive Clone

Panning to identify cross-reactive clones. Cross-reactive clones were enriched and identified by panning round 2 human TfR phage output against cynomolgus TfR.

Panning method: Detailed panning was carried out by panning against human TfR in round 1 and 2 and panning against cynomolgus TfR in round 3. Proteins were purchased from Sino Biologicals: huTfR 11020-H07H, cynomolgus TfR 90253-C07H. During the panning process, 0.1 mg/mL human transferrin (Tf, R&D 2914-HT) was included in the solution in order to block the phages that compete with Tf for binding TfR.

Detailed panning method: A phage-displayed scFv antibody library was prepared previously (S. Zhao et al., Partial Leptin Reduction as an Insulin Sensitization and Weight Loss Strategy. Cell metabolism 30, 706-719.e706 (2019)). Panning of the library for TfR specific antibodies was carried out as described previously with modifications. Briefly, MaxiSorp Nunc-Immuno tubes (Thermo Fisher Scientific) were coated with 20 μg/mL TfR proteins in DPBS overnight at 4° C. Unbound antigen was removed after washing with DPBS. After blocking the surface with 5% milk in DPBS, the phage library was incubated with the coated-TfR for 2 hours at room temperature in 5% milk. After washing with PBS+0.05% tween-20 to remove unbound phage, captured phage was eluted by incubating with 100 mM TEA for 20 min. Eluted phage-infected log-phase growing E. coli TG1 and then were amplified on 2× YTAG agar 500 cm² square plate (Corning) at 30° C. overnight. The amplified phage infected TG1 was used to prepare the phage for the next round of panning using the M13KO7 helper phage. The enrichment process was done in three rounds using the output from the previous round as the input for the next round.

Phage ELISA method: after three rounds of panning, the output titer was measured and single colonies were used to prepare phage for ELISA. High-binding ELISA plates (Corning) were coated with TfR at 2 μg/mL overnight at 4° C. After blocking with 5% milk in PBS, phage prepared from single TG1 colonies in 5% milk PBS was incubated with coated TfR for 1 hour at room temperature. After washing with PBS+0.05% Tween-20, anti-M13-HRP (Santa Cruz Biotechnology) was added at 1:2000 concentration and incubated for 1 hour at room temperature. After washing with PBS+0.05% Tween-20, TMB substrate (Thermo Fisher Scientific) was added and incubated for 5 min before being stopped by 1N H₂SO₄. OD values were read at 450 nm. Top 20% high-binding clones were selected. Phagemids were extracted using Qiagen BioRobot Universal System in 96-well format. After DNA sequencing, sequences were analyzed using the IMGT V-quest service to identify antibody sequences with unique CDR3 regions.

Panning results, phage ELISA: Cross-reactive clones were identified after round 3 panning. In the output, about 8% of the clones showed cross-reactivity to both human and cynomolgus TfR.

Panning results, unique clones: Then, all clones were sequenced and 18 unique antibody scFv sequences were identified.

Conversion into human IgG1 format: 18 unique IgG molecules were converted and produced in Expi293F cell. The proteins were purified using Protein A agarose beads.

Screening purified antibodies for binding TfR:

The purified antibodies were studied for binding human TfR ECD and Cyno TfR ECD using Octet.

Conversion into human IgG1 format: 18 unique IgG molecules were converted and produced in Expi293F cell. The proteins were purified using Protein A agarose beads.

Screening purified antibodies for binding TfR: The purified antibodies were studied for binding human TfR ECD and Cyno TfR ECD using Octet. Within the 18 candidates, 10 antibodies show binding to both human and cyno TfR ECD: 1B2, 1C8, 2C3, 3H8, 4G1, 5B6, 7A1, 7B10, 8A5, 8G5.

KD measurement of 4G1 and 5B6: From the 10 binding candidates identified, 4G1 and 5B6 were selected as the final candidates, and their KD values were further measured using a serial dilution of antigen concentrations (data in Table A). 4G1 and 5B6 have satisfactory KD values.

TABLE A
antibody binding kinetics profile of 4G1 and 5B6.

| Antigen | Antibody | K_{D} (M) | kₒₙ(1/Ms) | k_{dis}(1/s) | R² |
|---|---|---|---|---|---|
| HuTfR | 4G1 | <1.0E−12 | 7.99E+04 | <1.0E−07 | 0.9981 |
| HuTfR | 5B6 | 5.75E−10 | 5.18E+04 | 2.98E−05 | 0.9992 |
| CynoTfR | 4G1 | <1.0E−12 | 9.45E+04 | <1.0E−07 | 0.9971 |
| CynoTfR | 5B6 | <1.0E−12 | 8.79E+04 | <1.0E−07 | 0.9992 |

### LIST OF EMBODIMENTS

1. An isolated monoclonal antibody, wherein the antibody specifically binds to TfR and wherein the antibody competes for binding of the TfR epitopes with an antibody selected from the group consisting of 1B2, 1C8, 2C3, 3H8, 4G1, 5B6, 7A1, 7B10, 8A5, 8G5, mTfR-2, mTfR-4, mTfR5, mTfR-42, mTfR-59, hTfR-1, hTfR-1, hTfR-1, and hTfR-1.
2. The antibody of Embodiment 1, or antigen-binding fragment thereof, wherein the antibody comprises:
(a) a first V_{L} region at least 80% identical to 1B2 VL CDR1 IMGT (SEQ ID NO: 156), 1C8 VL CDR1 IMGT (SEQ ID NO: 158), 2C3 VL CDR1 IMGT (SEQ ID NO: 160), 3H8 VL CDR1 IMGT (SEQ ID NO: 162), 4G1 VLCDR1 IMGT (SEQ ID NO: 164), 5B6VL CDR1 IMGT (SEQ ID NO: 166), 7A1 VL CDR1 IMGT (SEQ ID NO: 168), 7B10 VL CDR1 IMGT (SEQ ID NO: 170), 8A5 VL CDR1 IMGT (SEQ ID NO: 172), 8G5 VL CDR1 IMGT (SEQ ID NO: 174), mTfR-2LCDR1-AA (SEQ ID NO: 28), mTfR-4LCDR1-AA (SEQ ID NO: 31), mTfR-5LCDR1-AA (SEQ ID NO: 34), mTfR-42LCDR1-AA (SEQ ID NO: 37), mTfR-59LCDR1-AA (SEQ ID NO: 40), hTfR-1LCDR1-AA (SEQ ID NO: 43), hTfR-20LCDR1-AA (SEQ ID NO: 46), hTfR-13LCDR1-AA (SEQ ID NO: 49), or hTfR-30LCDR1-AA (SEQ ID NO: 52);
(b) a second V_{L} CDR at least 80% identical to a tripeptide selected from the group consisting of QDS, KAS, AAS, GND, GTS, YDS, EVS, LGS, SNI, ANS, SNN, DDN, RNN, EDN, and DVS;
(c) a third V_{L} CDR at least 80% identical to 1B2 VL CDR3 IMGT (SEQ ID NO: 157), 1C8 VL CDR3 IMGT (SEQ ID NO: 159), 2C3 VL CDR3 IMGT (SEQ ID NO: 161), 3H8 L CDR3 IMGT (SEQ ID NO: 163), 4G1 VL CDR3 IMGT (SEQ ID NO: 165), 5B6 VL CDR3 IMGT (SEQ ID NO: 167), 7A1 VL CDR3 IMGT (SEQ ID NO: 169), 7B10 VL CDR3 IMGT (SEQ ID NO: 171), 8A5 VL CDR3 IMGT (SEQ ID NO: 173), 8G5 VL CDR3 IMGT (SEQ ID NO: 175), mTfR-2LCDR3-AA (SEQ ID NO: 30), mTfR-4LCDR3-AA (SEQ ID NO: 33), mTfR-5LCDR3-AA (SEQ ID NO: 36), mTfR-42LCDR3-AA (SEQ ID NO: 39), mTfR-59LCDR3-AA (SEQ ID NO: 42), hTfR-1LCDR3-AA (SEQ ID NO: 45), hTfR-20LCDR3-AA (SEQ ID NO: 48), hTfR-13LCDR3-AA (SEQ ID NO: 51), or hTfR-30LCDR3-AA (SEQ ID NO: 54);
(d) a first V_{H} CDR at least 80% identical to 1B2 VH CDR1 IMGT (SEQ ID NO: 126), 1C8 VH CDR1 IMGT (SEQ ID NO: 129), 2C3 VH CDR1 IMGT (SEQ ID NO: 132), 3H8 VH CDR1 IMGT (SEQ ID NO: 135), 4G1 VH CDR1 IMGT (SEQ ID NO: 138), 5B6 VH CDR1 IMGT (SEQ ID NO: 141), 7A1 VH CDR1 IMGT (SEQ ID NO: 144), 7B10 VH CDR1 IMGT (SEQ ID NO: 147), 8A5 VH CDR1 IMGT (SEQ ID NO: 150), 8G5 VH CDR1 IMGT (SEQ ID NO: 153), mTfR-2LHCDR1-AA (SEQ ID NO: 1), mTfR-4HCDR1-AA (SEQ ID NO: 4), mTfR-5HCDR1-AA (SEQ ID NO: 7), mTfR-42HCDR1-AA (SEQ ID NO: 10), mTfR-59HCDR1-AA (SEQ ID NO: 13), hTfR-1HCDR1-AA (SEQ ID NO: 16), hTfR-20HCDR1-AA (SEQ ID NO: 19), hTfR-13HCDR1-AA (SEQ ID NO: 22), or hTfR-30HCDR1-AA (SEQ ID NO: 25);
(e) a second V_{H} CDR at least 80% identical to 1B2 VH CDR2 IMGT (SEQ ID NO: 127), 1C8 VH CDR2 IMGT (SEQ ID NO: 130), 2C3 VH CDR2 IMGT (SEQ ID NO: 133), 3H8 VH CDR2 IMGT (SEQ ID NO: 136), 4G1 VH CDR2 IMGT (SEQ ID NO: 139), 5B6 VH CDR2 IMGT (SEQ ID NO: 142), 7A1 VH CDR2 IMGT (SEQ ID NO: 145), 7B10 VH CDR2 IMGT (SEQ ID NO: 148), 8A5 VH CDR2 IMGT (SEQ ID NO: 151), 8G5 VH CDR2 IMGT (SEQ ID NO: 154), mTfR-2LHCDR2-AA (SEQ ID NO: 2), mTfR-4HCDR2-AA (SEQ ID NO: 5), mTfR-5HCDR2-AA (SEQ ID NO: 8), mTfR-42-HCDR2-AA (SEQ ID NO: 11), mTfR-59HCDR2-AA (SEQ ID NO: 14), hTfR-1HCDR2-AA (SEQ ID NO: 17), hTfR-20HCDR2-AA (SEQ ID NO: 20), hTfR-13HCDR2-AA (SEQ ID NO: 23), or hTfR-30HCDR2-AA (SEQ ID NO: 26); and
(f) a third V_{H} CDR at least 80% identical to 1B2 VH CDR3 IMGT (SEQ ID NO: 28), 1C8 VH CDR3 IMGT (SEQ ID NO: 131), 2C3 VH CDR3 IMGT (SEQ ID NO: 134), 3H8 VH CDR3 IMGT (SEQ ID NO: 137), 4G1 VH CDR3 IMGT (SEQ ID NO: 140), 5B6 VH CDR3 IMGT (SEQ ID NO: 143), 7A1 VH CDR3 IMGT (SEQ ID NO: 146), 7B10 VH CDR3 IMGT (SEQ ID NO: 149), 8A5 VH CDR3 IMGT (SEQ ID NO: 152), 8G5 VH CDR3 IMGT (SEQ ID NO: 155), mTfR-2LHCDR3-AA (SEQ ID NO: 3), mTfR-4HCDR3-AA (SEQ ID NO: 6), mTfR-5CDR3-AA (SEQ ID NO: 9), mTfR-42HCDR3-AA (SEQ ID NO: 12), mTfR-59HCDR3-AA (SEQ ID NO: 15), hTfR-1HCDR3-AA (SEQ ID NO: 18), hTfR-20HCDR3-AA (SEQ ID NO: 21), hTfR-13HCDR3-AA (SEQ ID NO: 24), or hTfR-30HCDR3-AA (SEQ ID NO: 27).
   3. The isolated antibody of Embodiment 2, wherein the antibody comprises:
(a) a first V_{H} CDR is identical to (SEQ ID NO: 126);
(b) a second V_{H} CDR is identical to (SEQ ID NO: 127);
(c) a third V_{H} CDR is identical to (SEQ ID NO: 128);
(d) a first V_{L} CDR is identical to (SEQ ID NO: 156);
(e) a second V_{L} CDR is identical to the tripeptide QDS; and
(f) a third V_{L} CDR is identical to (SEQ ID NO: 157).
   4. The isolated antibody of Embodiment 2, wherein the antibody comprises:
(a) a first V_{H} CDR is identical to (SEQ ID NO: 129);
(b) a second V_{H} CDR is identical to (SEQ ID NO: 130);
(c) a third V_{H} CDR is identical to (SEQ ID NO: 131);
(d) a first V_{L} CDR is identical to (SEQ ID NO: 158);
(e) a second V_{L} CDR is identical to the tripeptide KAS; and
(f) a third V_{L} CDR is identical to (SEQ ID NO: 159).
   5. The isolated antibody of Embodiment 2, wherein the antibody comprises:
(a) a first V_{H} CDR is identical to (SEQ ID NO: 132);
(b) a second V_{H} CDR is identical to (SEQ ID NO: 133);
(c) a third V_{H} CDR is identical to (SEQ ID NO: 134);
(d) a first V_{L} CDR is identical to (SEQ ID NO: 160);
(e) a second V_{L} CDR is identical to the tripeptide AAS; and
(f) a third V_{L} CDR is identical to (SEQ ID NO: 161).
   6. The isolated antibody of Embodiment 2, wherein the antibody comprises:
(a) a first V_{H} CDR is identical to (SEQ ID NO: 135);
(b) a second V_{H} CDR is identical to (SEQ ID NO: 136);
(c) a third V_{H} CDR is identical to (SEQ ID NO: 137);
(d) a first V_{L} CDR is identical to (SEQ ID NO: 162);
(e) a second V_{L} CDR is identical to the tripeptide GND; and
(f) a third V_{L} CDR is identical to (SEQ ID NO: 163).
   7. The isolated antibody of Embodiment 2, wherein the antibody comprises:
(a) a first V_{H} CDR is identical to (SEQ ID NO: 138);
(b) a second V_{H} CDR is identical to (SEQ ID NO: 139);
(c) a third V_{H} CDR is identical to (SEQ ID NO: 140);
(d) a first V_{L} CDR is identical to (SEQ ID NO: 164);
(e) a second V_{L} CDR is identical to the tripeptide GTS; and
(f) a third V_{L} CDR is identical to (SEQ ID NO: 165).
   8. The isolated antibody of Embodiment 2, wherein the antibody comprises:
(a) a first V_{H} CDR is identical to (SEQ ID NO: 141);
(b) a second V_{H} CDR is identical to (SEQ ID NO: 142);
(c) a third V_{H} CDR is identical to (SEQ ID NO: 143);
(d) a first V_{L} CDR is identical to (SEQ ID NO: 166);
(e) a second V_{L} CDR is identical to the tripeptide YDS; and
(f) a third V_{L} CDR is identical to (SEQ ID NO: 167).
   9. The isolated antibody of Embodiment 2, wherein the antibody comprises:
(a) a first V_{H} CDR is identical to (SEQ ID NO: 144);
(b) a second V_{H} CDR is identical to (SEQ ID NO: 145);
(c) a third V_{H} CDR is identical to (SEQ ID NO: 146);
(d) a first V_{L} CDR is identical to (SEQ ID NO: 168);
(e) a second V_{L} CDR is identical to the tripeptide EVS; and
(f) a third V_{L} CDR is identical to (SEQ ID NO: 169).
   10. The isolated antibody of Embodiment 2, wherein the antibody comprises:
(a) a first V_{H} CDR is identical to (SEQ ID NO: 147);
(b) a second V_{H} CDR is identical to (SEQ ID NO: 148);
(c) a third V_{H} CDR is identical to (SEQ ID NO: 149);
(d) a first V_{L} CDR is identical to (SEQ ID NO: 170);
(e) a second V_{L} CDR is identical to the tripeptide LGS; and
(f) a third V_{L} CDR is identical to (SEQ ID NO: 171).
   11. The isolated antibody of Embodiment 2, wherein the antibody comprises:
(a) a first V_{H} CDR is identical to (SEQ ID NO: 150);
(b) a second V_{H} CDR is identical to (SEQ ID NO: 151);
(c) a third V_{H} CDR is identical to (SEQ ID NO: 152);
(d) a first V_{L} CDR is identical to (SEQ ID NO: 172);
(e) a second V_{L} CDR is identical to the tripeptide SNI; and
(f) a third V_{L} CDR is identical to (SEQ ID NO: 173).
   12. The isolated antibody of Embodiment 2, wherein the antibody comprises:
(a) a first V_{H} CDR is identical to (SEQ ID NO: 153);
(b) a second V_{H} CDR is identical to (SEQ ID NO: 154);
(c) a third V_{H} CDR is identical to (SEQ ID NO: 155);
(d) a first V_{L} CDR is identical to (SEQ ID NO: 174);
(e) a second V_{L} CDR is identical to the tripeptide ANS; and
(f) a third V_{L} CDR is identical to (SEQ ID NO: 175).
   13. The isolated antibody of Embodiment 2, wherein the antibody comprises:
(a) a first V_{H} CDR is identical to SEQ ID NO: 1;
(b) a second V_{H} CDR is identical to SEQ ID NO: 2;
(c) a third V_{H} CDR is identical to SEQ ID NO: 3;
(d) a first V_{L} CDR is identical to SEQ ID NO: 28;
(e) a second V_{L} CDR is identical to the tripeptide SNN; and
(f) a third V_{L} CDR is identical to SEQ ID NO: 29.
   14. The isolated antibody of Embodiment 2, wherein the antibody comprises:
(a) a first V_{H} CDR is identical to SEQ ID NO: 4;
(b) a second V_{H} CDR is identical to SEQ ID NO: 5;
(c) a third V_{H} CDR is identical to SEQ ID NO: 6;
(d) a first V_{L} CDR is identical to SEQ ID NO: 30;
(e) a second V_{L} CDR is identical to the tripeptide DDN; and
(f) a third V_{L} CDR is identical to SEQ ID NO: 31.
   15. The isolated antibody of Embodiment 2, wherein the antibody comprises:
(a) a first V_{H} CDR is identical to SEQ ID NO: 7;
(b) a second V_{H} CDR is identical to SEQ ID NO: 8;
(c) a third V_{H} CDR is identical to SEQ ID NO: 9;
(d) a first V_{L} CDR is identical to SEQ ID NO: 32;
(e) a second V_{L} CDR is identical to the tripeptide RNN; and
(f) a third V_{L} CDR is identical to SEQ ID NO: 33.
   16. The isolated antibody of Embodiment 2, wherein the antibody comprises:
(a) a first V_{H} CDR is identical to SEQ ID NO: 10;
(b) a second V_{H} CDR is identical to SEQ ID NO: 11;
(c) a third V_{H} CDR is identical to SEQ ID NO: 12;
(d) a first V_{L} CDR is identical to SEQ ID NO: 34;
(e) a second V_{L} CDR is identical to the tripeptide AAS; and
(f) a third V_{L} CDR is identical to SEQ ID NO: 35.
   17. The isolated antibody of Embodiment 2, wherein the antibody comprises:
(a) a first V_{H} CDR is identical to SEQ ID NO: 13;
(b) a second V_{H} CDR is identical to SEQ ID NO: 14;
(c) a third V_{H} CDR is identical to SEQ ID NO: 15;
(d) a first V_{L} CDR is identical to SEQ ID NO: 36;
(e) a second V_{L} CDR is identical to the tripeptide EDN; and
(f) a third V_{L} CDR is identical to SEQ ID NO: 37.
   18. The isolated antibody of Embodiment 2, wherein the antibody comprises:
(a) a first V_{H} CDR is identical to SEQ ID NO: 16;
(b) a second V_{H} CDR is identical to SEQ ID NO: 17;
(c) a third V_{H} CDR is identical to SEQ ID NO: 18;
(d) a first V_{L} CDR is identical to SEQ ID NO: 38;
(e) a second V_{L} CDR is identical to the tripeptide DVS; and
(f) a third V_{L} CDR is identical to SEQ ID NO: 39.
   19. The isolated antibody of Embodiment 2, wherein the antibody comprises:
(a) a first V_{H} CDR is identical to SEQ ID NO: 19;
(b) a second V_{H} CDR is identical to SEQ ID NO: 20;
(c) a third V_{H} CDR is identical to SEQ ID NO: 21;
(d) a first V_{L} CDR is identical to SEQ ID NO: 40;
(e) a second V_{L} CDR is identical to the tripeptide AAS; and
(f) a third V_{L} CDR is identical to SEQ ID NO: 41.
   20. The isolated antibody of Embodiment 2, wherein the antibody comprises:
(a) a first V_{H} CDR is identical to SEQ ID NO: 22;
(b) a second V_{H} CDR is identical to SEQ ID NO: 23;
(c) a third V_{H} CDR is identical to SEQ ID NO: 24;
(d) a first V_{L} CDR is identical to SEQ ID NO: 42;
(e) a second V_{L} CDR is identical to the tripeptide AAS; and
(f) a third V_{L} CDR is identical to SEQ ID NO: 43.
   21. The isolated antibody of Embodiment 2, wherein the antibody comprises:
(a) a first V_{H} CDR is identical to SEQ ID NO: 25;
(b) a second V_{H} CDR is identical to SEQ ID NO: 26;
(c) a third V_{H} CDR is identical to SEQ ID NO: 27;
(d) a first V_{L} CDR is identical to SEQ ID NO: 44;
(e) a second V_{L} CDR is identical to the tripeptide EVS; and
(f) a third V_{L} CDR is identical to SEQ ID NO: 45.
   22. The antibody of Embodiment 2, wherein the antibody comprises:
(i) a V_{H} domain at least about 80% identical to the V_{H} domain of 1B2 or the humanized V_{H} domain of 1B2 amino acid sequence; and a V_{L} domain at least about 80% identical to the V_{L} domain of 1B2 or the humanized V_{L} domain of the 1B2 amino acid sequence;
(ii) a V_{H} domain at least about 80% identical to the V_{H} domain of 1C8 or the humanized V_{H} domain of 1C8 amino acid sequence; and a V_{L} domain at least about 80% identical to the V_{L} domain of 1C8 or the humanized V_{L} domain of the 1C8 amino acid sequence;
(iii) a V_{H} domain at least about 80% identical to the V_{H} domain of 2C3 or the humanized V_{H} domain of 2C3 amino acid sequence; and a V_{L} domain at least about 80% identical to the V_{L} domain of 2C3 or the humanized V_{L} domain of the 2C3 amino acid sequence;
(iv) a V_{H} domain at least about 80% identical to the V_{H} domain of 3H8 or the humanized V_{H} domain of 3H8 amino acid sequence; and a V_{L} domain at least about 80% identical to the V_{L} domain of 3H8 or the humanized V_{L} domain of the 3H8 amino acid sequence;
(v) a V_{H} domain at least about 80% identical to the V_{H} domain of 4G1 or the humanized V_{H} domain of 4G1 amino acid sequence; and a V_{L} domain at least about 80% identical to the V_{L} domain of 4G1 or the humanized V_{L} domain of the 4G1 amino acid sequence;
(vi) a V_{H} domain at least about 80% identical to the V_{H} domain of 5B6 or the humanized V_{H} domain of 5B6 amino acid sequence; and a V_{L} domain at least about 80% identical to the V_{L} domain of 5B6 or the humanized V_{L} domain of the 5B6 amino acid sequence;
(vii) a V_{H} domain at least about 80% identical to the V_{H} domain of 7A1 or the humanized V_{H} domain of 7A1 amino acid sequence; and a V_{L} domain at least about 80% identical to the V_{L} domain of 7A1 or the humanized V_{L} domain of the 7A1 amino acid sequence;
(viii) a V_{H} domain at least about 80% identical to the V_{H} domain of 7B10 or the humanized V_{H} domain of 7B10 amino acid sequence; and a V_{L} domain at least about 80% identical to the V_{L} domain of 7B10 or the humanized V_{L} domain of the 7B10 amino acid sequence;
(ix) a V_{H} domain at least about 80% identical to the V_{H} domain of 8A5 or the humanized V_{H} domain of 8A5 amino acid sequence; and a V_{L} domain at least about 80% identical to the V_{L} domain of 8A5 or the humanized V_{L} domain of the 8A5 amino acid sequence;
(x) a V_{H} domain at least about 80% identical to the V_{H} domain of 8G5 amino acid sequence or the humanized V_{H} domain of 8G5 amino acid sequence; and a V_{L} domain at least about 80% identical to the V_{L} domain of 8G5 amino acid sequence or the humanized V_{L} domain of 8G5 VL amino acid sequence;
(xi) a V_{H} domain at least about 80% identical to the V_{H} domain of mTfR-2H-AA (SEQ ID NO: 46) or the humanized V_{H} domain of mTfR-2H-AA; and a V_{L} domain at least about 80% identical to the V_{L} domain of mTfR-2L-AA (SEQ ID NO: 55) or the humanized V_{L} domain of mTfR-2L-AA;
(xii) a V_{H} domain at least about 80% identical to the V_{H} domain of mTfR-4H-AA (SEQ ID NO: 47) or the humanized V_{H} domain of mTfR-4H-AA; and a V_{L} domain at least about 80% identical to the V_{L} domain of mTfR-4L-AA (SEQ ID NO: 56) or the humanized V_{L} domain of mTfR-4L-AA;
(xiii) a V_{H} domain at least about 80% identical to the V_{H} domain of mTfR-5H-AA (SEQ ID NO: 48) or the humanized V_{H} domain of mTfR-5H-AA; and a V_{L} domain at least about 80% identical to the V_{L} domain of mTfR-5L-AA (SEQ ID NO: 57) or the humanized V_{L} domain of mTfR-5L-AA;
(xiv) a V_{H} domain at least about 80% identical to the V_{H} domain of mTfR-42H-AA (SEQ ID NO: 49) or the humanized V_{H} domain of mTfR-42H-AA; and a V_{L} domain at least about 80% identical to the V_{L} domain of mTfR-42L-AA (SEQ ID NO: 58) or the humanized V_{L} domain of mTfR-42L-AA;
(xv) a V_{H} domain at least about 80% identical to the V_{H} domain of mTfR-59H-AA (SEQ ID NO: 50) or the humanized V_{H} domain of mTfR-59H-AA; and a V_{L} domain at least about 80% identical to the V_{L} domain of mTfR-59L-AA (SEQ ID NO: 59) or the humanized V_{L} domain of mTfR-59L-AA;
(xvi) a V_{H} domain at least about 80% identical to the V_{H} domain of hTfR-1H-AA (SEQ ID NO: 51) or the humanized V_{H} domain of hTfR-1H-AA; and a V_{L} domain at least about 80% identical to the V_{L} domain of hTfR-1L-AA (SEQ ID NO: 60) or the humanized V_{L} domain of hTfR-1L-AA;
(xvii) a V_{H} domain at least about 80% identical to the V_{H} domain of hTfR-20H-AA (SEQ ID NO: 52) or the humanized V_{H} domain of hTfR-20H-AA; and a V_{L} domain at least about 80% identical to the V_{L} domain of hTfR-20L-AA (SEQ ID NO: 61) or the humanized V_{L} domain of hTfR-20L-AA;
(xviii) a V_{H} domain at least about 80% identical to the V_{H} domain of hTfR-13H-AA (SEQ ID NO: 53) or the humanized V_{H} domain of hTfR-13H-AA; and a V_{L} domain at least about 80% identical to the V_{L} domain of hTfR-13L-AA (SEQ ID NO: 62) or the humanized V_{L} domain of hTfR-13L-AA; or
(xix) a V_{H} domain at least about 80% identical to the V_{H} domain of hTfR-30H-AA (SEQ ID NO: 54) or the humanized V_{H} domain of hTfR-30H-AA; and a V_{L} domain at least about 80% identical to the V_{L} domain of hTfR-30L-AA (SEQ ID NO: 63) or the humanized V_{L} domain of hTfR-30L-AA.
   23. The antibody of any one of Embodiments 1-22, wherein the antibody is recombinant.
   24. The antibody of any one of Embodiments 1-22, wherein the antibody is an IgG, IgM, IgA or an antigen binding fragment thereof.
   25. The antibody of any one of Embodiments 1-22, wherein the antibody is a Fab′, a F(ab′)2, a F(ab′)3, a monovalent scFv, a bivalent scFv, or a single domain antibody.
   26. The antibody of any one of Embodiments 1-22, wherein the antibody is a human, humanized antibody or de-immunized antibody.
   27. The antibody of any one of Embodiments 1-22, wherein the antibody is conjugated to an imaging agent.
   28. A chimeric antigen receptor comprising an antigen-binding domain at least 80% identical to an antigen-binding domain of the monoclonal antibody of any one of the preceding Embodiments.
   29. A composition comprising an antibody of any one of Embodiments 1-28 in a pharmaceutically acceptable carrier.
   30. An isolated polynucleotide molecule comprising a nucleic acid sequence encoding an antibody of any one of Embodiments 1-27.
   31. A recombinant polypeptide comprising an antibody V_{H} domain comprising CDRs 1-3 of the V_{H} domain of; CDRs 1-3 of the V_{H} domain of 1B2; CDRs 1-3 of the V_{H} domain of 1C8; CDRs 1-3 of the V_{H} domain of 2C3; CDRs 1-3 of the V_{H} domain of 3H8; CDRs 1-3 of the V_{H} domain of 4G1; CDRs 1-3 of the V_{H} domain of 5B6; CDRs 1-3 of the V_{H} domain of 7A1; CDRs 1-3 of the V_{H} domain of 7B10; CDRs 1-3 of the V_{H} domain of 8A5 or CDRs 1-3 of the V_{H} domain of 8G5.
   32. A recombinant polypeptide comprising an antibody V_{L} domain comprising CDRs 1-3 of the V_{L} domain of 1B2; CDRs 1-3 of the V_{L} domain of 1C8; CDRs 1-3 of the V_{L} domain of 2C3; CDRs 1-3 of the V_{L} domain of 3H8; CDRs 1-3 of the V_{L} domain of 4G1; CDRs 1-3 of the V_{L} domain of 5B6; CDRs 1-3 of the V_{L} domain of 7A1; CDRs 1-3 of the V_{L} domain of 7B10; CDRs 1-3 of the V_{L} domain 8A5; or CDRs 1-3 of the V_{L} domain 8G5.
   33. An isolated polynucleotide molecule comprising a nucleic acid sequence encoding a polypeptide of Embodiment 30 or 31.
   34. A host cell comprising one or more polynucleotide molecule(s) encoding an antibody of any one of Embodiments 1-28 or a recombinant polypeptide of Embodiment 30 or 31.
   35. The host cell of Embodiment 34, wherein the host cell is a mammalian cell, a yeast cell, a bacterial cell, a ciliate cell or an insect cell.
   36. An expression vector comprising a polynucleotide having at least 95% identity to the nucleic acid sequence that encodes the variable region of the heavy chain 1B2 (SEQ ID NO: 176), 1C8 (SEQ ID NO: 177), 2C3 (SEQ ID NO: 178), 3H8 (SEQ ID NO: 179), 4G1 (SEQ ID NO: 180), 5B6 (SEQ ID NO: 181), 7A1 (SEQ ID NO: 182), 7B10 (SEQ ID NO: 183), 8A5 (SEQ ID NO: 184), 8G5 (SEQ ID NO: 185 mTfR-2H (SEQ ID NO: 46), mTfR-4H (SEQ ID NO: 47), mTfR-5H (SEQ ID NO: 48), mTfR-42H (SEQ ID NO: 49), mTfR-59H (SEQ ID NO: 50), hTfR-1H (SEQ ID NO: 51), hTfR-20H (SEQ ID NO: 52), hTfR-13H (SEQ ID NO: 53), or hTfR-30H (SEQ ID NO: 54).
   37. A method of manufacturing an antibody comprising:
(a) expressing one or more polynucleotide molecule(s) encoding a V_{L} and V_{H} chain of an antibody of any one of Embodiments 1-22 in a cell; and
(b) purifying the antibody from the cell and/or a fluid medium in which the cell is disposed.
   38. A method for treating a subject having a neurologic disorder or brain cancer comprising administering an effective amount of an antibody of any one of Embodiments 1-27 to the subject.
   39. The method of Embodiment 38, wherein the antibody is in a pharmaceutically suitable composition.
   40. The method of Embodiment 38, wherein the antibody is administered systemically.
   41. The method of Embodiment 38, wherein the antibody is administered intravenously, intradermally, intratumorally, intramuscularly, intraperitoneally, subcutaneously, intrathecally or locally.
   42. An isolated bispecific antibody, wherein a portion of the bispecific antibody specifically binds to TfR.
   43. The bispecific antibody of Embodiment 42, wherein the bispecific antibody specifically binds to VEGF-Trap fused with a monovalent TfR, wherein the antibody can be endocytosed by vascular epithelial cells in vivo without causing undue induction of TfR degradation.
   44. The bispecific antibody of Embodiment 42 or Embodiment 43, wherein the antibody can facilitate in vivo an attainment of VEGF concentrations in brain tissue that are greater than 10-fold higher than VEGF concentrations outside the brain tissue.
   45. The bispecific antibody of any one of Embodiments 42 through 44, wherein at least the antibody is derived from a murine antibody.
   46. The bispecific antibody of any one of Embodiments 42 through 45, wherein at least the antibody is derived from a human antibody.

1-26. (canceled)

27. An isolated monoclonal antibody, wherein the antibody specifically binds to TfR and wherein the antibody competes for binding of the TfR epitopes with an antibody selected from the group consisting of 1B2, 1C8, 2C3, 3H8, 4G1, 5B6, 7A1, 7B10, 8A5, 8G5, mTfR-2, mTfR-4, mTfR5, mTfR-42, mTfR-59, hTfR-1, hTfR-1, hTfR-1, and hTfR-1.

28. The antibody of claim 27, or antigen-binding fragment thereof, wherein the antibody comprises:
(a) a first V_{L} region at least 80% identical to 1B2 VL CDR1 IMGT (SEQ ID NO: 156), 1C8 VL CDR1 IMGT (SEQ ID NO: 158), 2C3 VL CDR1 IMGT (SEQ ID NO: 160), 3H8 VL CDR1 IMGT (SEQ ID NO: 162), 4G1 VLCDR1 IMGT (SEQ ID NO: 164), 5B6VL CDR1 IMGT (SEQ ID NO: 166), 7A1 VL CDR1 IMGT (SEQ ID NO: 168), 7B10 VL CDR1 IMGT (SEQ ID NO: 170), 8A5 VL CDR1 IMGT (SEQ ID NO: 172), 8G5 VL CDR1 IMGT (SEQ ID NO: 174), mTfR-2LCDR1-AA (SEQ ID NO: 28), mTfR-4LCDR1-AA (SEQ ID NO: 31), mTfR-5LCDR1-AA (SEQ ID NO: 34), mTfR-42LCDR1-AA (SEQ ID NO: 37), mTfR-59LCDR1-AA (SEQ ID NO: 40), hTfR-1LCDR1-AA (SEQ ID NO: 43), hTfR-20LCDR1-AA (SEQ ID NO: 46), hTfR-13LCDR1-AA (SEQ ID NO: 49), or hTfR-30LCDR1-AA (SEQ ID NO: 52);
(b) a second V_{L} CDR at least 80% identical to a tripeptide selected from the group consisting of QDS, KAS, AAS, GND, GTS, YDS, EVS, LGS, SNI, ANS, SNN, DDN, RNN, EDN, and DVS;
(c) a third V_{L} CDR at least 80% identical to 1B2 VL CDR3 IMGT (SEQ ID NO: 157), 1C8 VL CDR3 IMGT (SEQ ID NO: 159), 2C3 VL CDR3 IMGT (SEQ ID NO: 161), 3H8 L CDR3 IMGT (SEQ ID NO: 163), 4G1 VL CDR3 IMGT (SEQ ID NO: 165), 5B6 VL CDR3 IMGT (SEQ ID NO: 167), 7A1 VL CDR3 IMGT (SEQ ID NO: 169), 7B10 VL CDR3 IMGT (SEQ ID NO: 171), 8A5 VL CDR3 IMGT (SEQ ID NO: 173), 8G5 VL CDR3 IMGT (SEQ ID NO: 175), mTfR-2LCDR3-AA (SEQ ID NO: 30), mTfR-4LCDR3-AA (SEQ ID NO: 33), mTfR-5LCDR3-AA (SEQ ID NO: 36), mTfR-42LCDR3-AA (SEQ ID NO: 39), mTfR-59LCDR3-AA (SEQ ID NO: 42), hTfR-1LCDR3-AA (SEQ ID NO: 45), hTfR-20LCDR3-AA (SEQ ID NO: 48), hTfR-13LCDR3-AA (SEQ ID NO: 51), or hTfR-30LCDR3-AA (SEQ ID NO: 54);
(d) a first V_{H} CDR at least 80% identical to 1B2 VH CDR1 IMGT (SEQ ID NO: 126), 1C8 VH CDR1 IMGT (SEQ ID NO: 129), 2C3 VH CDR1 IMGT (SEQ ID NO: 132), 3H8 VH CDR1 IMGT (SEQ ID NO: 135), 4G1 VH CDR1 IMGT (SEQ ID NO: 138), 5B6 VH CDR1 IMGT (SEQ ID NO: 141), 7A1 VH CDR1 IMGT (SEQ ID NO: 144), 7B10 VH CDR1 IMGT (SEQ ID NO: 147), 8A5 VH CDR1 IMGT (SEQ ID NO: 150), 8G5 VH CDR1 IMGT (SEQ ID NO: 153), mTfR-2LHCDR1-AA (SEQ ID NO: 1), mTfR-4HCDR1-AA (SEQ ID NO: 4), mTfR-5HCDR1-AA (SEQ ID NO: 7), mTfR-42HCDR1-AA (SEQ ID NO: 10), mTfR-59HCDR1-AA (SEQ ID NO: 13), hTfR-1HCDR1-AA (SEQ ID NO: 16), hTfR-20HCDR1-AA (SEQ ID NO: 19), hTfR-13HCDR1-AA (SEQ ID NO: 22), or hTfR-30HCDR1-AA (SEQ ID NO: 25);
(e) a second V_{H} CDR at least 80% identical to 1B2 VH CDR2 IMGT (SEQ ID NO: 127), 1C8 VH CDR2 IMGT (SEQ ID NO: 130), 2C3 VH CDR2 IMGT (SEQ ID NO: 133), 3H8 VH CDR2 IMGT (SEQ ID NO: 136), 4G1 VH CDR2 IMGT (SEQ ID NO: 139), 5B6 VH CDR2 IMGT (SEQ ID NO: 142), 7A1 VH CDR2 IMGT (SEQ ID NO: 145), 7B10 VH CDR2 IMGT (SEQ ID NO: 148), 8A5 VH CDR2 IMGT (SEQ ID NO: 151), 8G5 VH CDR2 IMGT (SEQ ID NO: 154), mTfR-2LHCDR2-AA (SEQ ID NO: 2), mTfR-4HCDR2-AA (SEQ ID NO: 5), mTfR-5HCDR2-AA (SEQ ID NO: 8), mTfR-42-HCDR2-AA (SEQ ID NO: 11), mTfR-59HCDR2-AA (SEQ ID NO: 14), hTfR-1HCDR2-AA (SEQ ID NO: 17), hTfR-20HCDR2-AA (SEQ ID NO: 20), hTfR-13HCDR2-AA (SEQ ID NO: 23), or hTfR-30HCDR2-AA (SEQ ID NO: 26); and
(f) a third V_{H} CDR at least 80% identical to 1B2 VH CDR3 IMGT (SEQ ID NO: 28), 1C8 VH CDR3 IMGT (SEQ ID NO: 131), 2C3 VH CDR3 IMGT (SEQ ID NO: 134), 3H8 VH CDR3 IMGT (SEQ ID NO: 137), 4G1 VH CDR3 IMGT (SEQ ID NO: 140), 5B6 VH CDR3 IMGT (SEQ ID NO: 143), 7A1 VH CDR3 IMGT (SEQ ID NO: 146), 7B10 VH CDR3 IMGT (SEQ ID NO: 149), 8A5 VH CDR3 IMGT (SEQ ID NO: 152), 8G5 VH CDR3 IMGT (SEQ ID NO: 155), mTfR-2LHCDR3-AA (SEQ ID NO: 3), mTfR-4HCDR3-AA (SEQ ID NO: 6), mTfR-5CDR3-AA (SEQ ID NO: 9), mTfR-42HCDR3-AA (SEQ ID NO: 12), mTfR-59HCDR3-AA (SEQ ID NO: 15), hTfR-1HCDR3-AA (SEQ ID NO: 18), hTfR-20HCDR3-AA (SEQ ID NO: 21), hTfR-13HCDR3-AA (SEQ ID NO: 24), or hTfR-30HCDR3-AA (SEQ ID NO: 27).

29. The antibody of claim 28, wherein the antibody comprises:
(i) a V_{H} domain at least about 80% identical to the V_{H} domain of 1B2 or the humanized V_{H} domain of 1B2 amino acid sequence; and a V_{L} domain at least about 80% identical to the V_{L} domain of 1B2 or the humanized V_{L} domain of the 1B2 amino acid sequence;
(ii) a V_{H} domain at least about 80% identical to the V_{H} domain of 1C8 or the humanized V_{H} domain of 1C8 amino acid sequence; and a V_{L} domain at least about 80% identical to the V_{L} domain of 1C8 or the humanized V_{L} domain of the 1C8 amino acid sequence;
(iii) a V_{H} domain at least about 80% identical to the V_{H} domain of 2C3 or the humanized V_{H} domain of 2C3 amino acid sequence; and a V_{L} domain at least about 80% identical to the V_{L} domain of 2C3 or the humanized V_{L} domain of the 2C3 amino acid sequence;
(iv) a V_{H} domain at least about 80% identical to the V_{H} domain of 3H8 or the humanized V_{H} domain of 3H8 amino acid sequence; and a V_{L} domain at least about 80% identical to the V_{L} domain of 3H8 or the humanized V_{L} domain of the 3H8 amino acid sequence;
(v) a V_{H} domain at least about 80% identical to the V_{H} domain of 4G1 or the humanized V_{H} domain of 4G1 amino acid sequence; and a V_{L} domain at least about 80% identical to the V_{L} domain of 4G1 or the humanized V_{L} domain of the 4G1 amino acid sequence;
(vi) a V_{H} domain at least about 80% identical to the V_{H} domain of 5B6 or the humanized V_{H} domain of 5B6 amino acid sequence; and a V_{L} domain at least about 80% identical to the V_{L} domain of 5B6 or the humanized V_{L} domain of the 5B6 amino acid sequence;
(vii) a V_{H} domain at least about 80% identical to the V_{H} domain of 7A1 or the humanized V_{H} domain of 7A1 amino acid sequence; and a V_{L} domain at least about 80% identical to the V_{L} domain of 7A1 or the humanized V_{L} domain of the 7A1 amino acid sequence;
(viii) a V_{H} domain at least about 80% identical to the V_{H} domain of 7B10 or the humanized V_{H} domain of 7B10 amino acid sequence; and a V_{L} domain at least about 80% identical to the V_{L} domain of 7B10 or the humanized V_{L} domain of the 7B10 amino acid sequence;
(ix) a V_{H} domain at least about 80% identical to the V_{H} domain of 8A5 or the humanized V_{H} domain of 8A5 amino acid sequence; and a V_{L} domain at least about 80% identical to the V_{L} domain of 8A5 or the humanized V_{L} domain of the 8A5 amino acid sequence;
(x) a V_{H} domain at least about 80% identical to the V_{H} domain of 8G5 amino acid sequence or the humanized V_{H} domain of 8G5 amino acid sequence; and a V_{L} domain at least about 80% identical to the V_{L} domain of 8G5 amino acid sequence or the humanized V_{L} domain of 8G5 V_{L} amino acid sequence;
(xi) a V_{H} domain at least about 80% identical to the V_{H} domain of mTfR-2H-AA (SEQ ID NO: 46) or the humanized V_{H} domain of mTfR-2H-AA; and a V_{L} domain at least about 80% identical to the V_{L} domain of mTfR-2L-AA (SEQ ID NO: 55) or the humanized V_{L} domain of mTfR-2L-AA;
(xii) a V_{H} domain at least about 80% identical to the V_{H} domain of mTfR-4H-AA (SEQ ID NO: 47) or the humanized V_{H} domain of mTfR-4H-AA; and a V_{L} domain at least about 80% identical to the V_{L} domain of mTfR-4L-AA (SEQ ID NO: 56) or the humanized V_{L} domain of mTfR-4L-AA;
(xiii) a V_{H} domain at least about 80% identical to the V_{H} domain of mTfR-5H-AA (SEQ ID NO: 48) or the humanized V_{H} domain of mTfR-5H-AA; and a V_{L} domain at least about 80% identical to the V_{L} domain of mTfR-5L-AA (SEQ ID NO: 57) or the humanized V_{L} domain of mTfR-5L-AA;
(xiv) a V_{H} domain at least about 80% identical to the V_{H} domain of mTfR-42H-AA (SEQ ID NO: 49) or the humanized V_{H} domain of mTfR-42H-AA; and a V_{L} domain at least about 80% identical to the V_{L} domain of mTfR-42L-AA (SEQ ID NO: 58) or the humanized V_{L} domain of mTfR-42L-AA;
(xv) a V_{H} domain at least about 80% identical to the V_{H} domain of mTfR-59H-AA (SEQ ID NO: 50) or the humanized V_{H} domain of mTfR-59H-AA; and a V_{L} domain at least about 80% identical to the V_{L} domain of mTfR-59L-AA (SEQ ID NO: 59) or the humanized V_{L} domain of mTfR-59L-AA;
(xvi) a V_{H} domain at least about 80% identical to the V_{H} domain of hTfR-1H-AA (SEQ ID NO: 51) or the humanized V_{H} domain of hTfR-1H-AA; and a V_{L} domain at least about 80% identical to the V_{L} domain of hTfR-1L-AA (SEQ ID NO: 60) or the humanized V_{L} domain of hTfR-1L-AA;
(xvii) a V_{H} domain at least about 80% identical to the V_{H} domain of hTfR-20H-AA (SEQ ID NO: 52) or the humanized V_{H} domain of hTfR-20H-AA; and a V_{L} domain at least about 80% identical to the V_{L} domain of hTfR-20L-AA (SEQ ID NO: 61) or the humanized V_{L} domain of hTfR-20L-AA;
(xviii) a V_{H} domain at least about 80% identical to the V_{H} domain of hTfR-13H-AA (SEQ ID NO: 53) or the humanized V_{H} domain of hTfR-13H-AA; and a V_{L} domain at least about 80% identical to the V_{L} domain of hTfR-13L-AA (SEQ ID NO: 62) or the humanized V_{L} domain of hTfR-13L-AA; or
(xix) a V_{H} domain at least about 80% identical to the V_{H} domain of hTfR-30H-AA (SEQ ID NO: 54) or the humanized V_{H} domain of hTfR-30H-AA; and a V_{L} domain at least about 80% identical to the V_{L} domain of hTfR-30L-AA (SEQ ID NO: 63) or the humanized V_{L} domain of hTfR-30L-AA.

30. The antibody of any one of claims 27-29, wherein the antibody is recombinant.

31. The antibody of any one of claims 27-30, wherein the antibody is an IgG, IgM, IgA or an antigen binding fragment thereof.

32. The antibody of any one of claims 27-31, wherein the antibody is a Fab′, a F(ab′)2, a F(ab′)3, a monovalent scFv, a bivalent scFv, or a single domain antibody.

33. The antibody of any one of claims 27-32, wherein the antibody is a human, humanized antibody or de-immunized antibody.

34. The antibody of any one of claims 27-33, wherein the antibody is conjugated to an imaging agent.

35. A chimeric antigen receptor comprising an antigen-binding domain at least 80% identical to an antigen-binding domain of the monoclonal antibody of any one of claims 27-34.

36. A composition comprising an antibody of any one of claims 27-34 in a pharmaceutically acceptable carrier.

37. An isolated polynucleotide molecule comprising a nucleic acid sequence encoding an antibody of any one of claims 27-34.

38. A recombinant polypeptide comprising an antibody V_{H} domain comprising CDRs 1-3 of the V_{H} domain of; CDRs 1-3 of the V_{H} domain of 1B2; CDRs 1-3 of the V_{H} domain of 1C8; CDRs 1-3 of the V_{H} domain of 2C3; CDRs 1-3 of the V_{H} domain of 3H8; CDRs 1-3 of the V_{H} domain of 4G1; CDRs 1-3 of the V_{H} domain of 5B6; CDRs 1-3 of the V_{H} domain of 7A1; CDRs 1-3 of the V_{H} domain of 7B10; CDRs 1-3 of the V_{H} domain of 8A5 or CDRs 1-3 of the V_{H} domain of 8G5.

39. A recombinant polypeptide comprising an antibody V_{L} domain comprising CDRs 1-3 of the V_{L} domain of 1B2; CDRs 1-3 of the V_{L} domain of 1C8; CDRs 1-3 of the V_{L} domain of 2C3; CDRs 1-3 of the V_{L} domain of 3H8; CDRs 1-3 of the V_{L} domain of 4G1; CDRs 1-3 of the V_{L} domain of 5B6; CDRs 1-3 of the V_{L} domain of 7A1; CDRs 1-3 of the V_{L} domain of 7B10; CDRs 1-3 of the V_{L} domain 8A5; or CDRs 1-3 of the V_{L} domain 8G5.

40. An isolated polynucleotide molecule comprising a nucleic acid sequence encoding a polypeptide of claim 38 or 39.

41. A host cell comprising one or more polynucleotide molecule(s) encoding an antibody of any one of claims 27-34 or a recombinant polypeptide of claim 38 or 39.

42. The host cell of claim 41, wherein the host cell is a mammalian cell, a yeast cell, a bacterial cell, a ciliate cell or an insect cell.

43. An expression vector comprising a polynucleotide having at least 95% identity to the nucleic acid that encodes the variable region of the heavy chain polypeptide of 1B2 (SEQ ID NO: 176), 1C8 (SEQ ID NO: 177), 2C3 (SEQ ID NO: 178), 3H8 (SEQ ID NO: 179), 4G1 (SEQ ID NO: 180), 5B6 (SEQ ID NO: 181), 7A1 (SEQ ID NO: 182), 7B10 (SEQ ID NO: 183), 8A5 (SEQ ID NO: 184), 8G5 (SEQ ID NO: 185 mTfR-2H (SEQ ID NO: 46), mTfR-4H (SEQ ID NO: 47), mTfR-5H (SEQ ID NO: 48), mTfR-42H (SEQ ID NO: 49), mTfR-59H (SEQ ID NO: 50), hTfR-1H (SEQ ID NO: 51), hTfR-20H (SEQ ID NO: 52), hTfR-13H (SEQ ID NO: 53), or hTfR-30H (SEQ ID NO: 54).

44. A method of manufacturing an antibody comprising:
(a) expressing one or more polynucleotide molecule(s) encoding a V_{L} and V_{H} chain of an antibody of any one of claims 27-34 in a cell; and
(b) purifying the antibody from the cell and/or a fluid medium in which the cell is disposed.

45. A method for treating a subject having a neurologic disorder or brain cancer comprising administering an effective amount of an antibody of any one of claims 27-34 to the subject.

46. The method of claim 45, wherein the antibody is in a pharmaceutically suitable composition.

47. The method of claim 45 or 46, wherein the antibody is administered systemically.

48. The method of claim 45 or 46, wherein the antibody is administered intravenously, intradermally, intratumorally, intramuscularly, intraperitoneally, subcutaneously, intrathecally or locally.

49. An isolated bispecific antibody, wherein a portion of the bispecific antibody specifically binds to TfR.

50. The bispecific antibody of claim 49, wherein the bispecific antibody specifically binds to VEGF-Trap fused with a monovalent TfR, wherein the antibody can be endocytosed by vascular epithelial cells in vivo without causing undue induction of TfR degradation.

51. The bispecific antibody of claim 49 or 50, wherein the antibody can facilitate in vivo an attainment of VEGF concentrations in brain tissue that are greater than 10-fold higher than VEGF concentrations outside the brain tissue.

52. The bispecific antibody of any one of claims 49-51, wherein at least the antibody is derived from a murine antibody.

53. The bispecific antibody of any one of claims 49-52, wherein at least the antibody is derived from a human antibody.


SEQUENCE LISTING (410 sequences)

SEQ ID NO: 1 - protein, 8 aa, Homo sapiens
GGTFSSYA

SEQ ID NO: 2 - protein, 8 aa, Homo sapiens
IIPIFGTA

SEQ ID NO: 3 - protein, 13 aa, Homo sapiens
ARFAFFSLASEDY

SEQ ID NO: 4 - protein, 8 aa, Homo sapiens
GFTFSWYT

SEQ ID NO: 5 - protein, 8 aa, Homo sapiens
ISSSSSDM

SEQ ID NO: 6 - protein, 19 aa, Homo sapiens
ARAHYYDSTSLNHFYGMDV

SEQ ID NO: 7 - protein, 8 aa, Homo sapiens
GFTFDDYA

SEQ ID NO: 8 - protein, 8 aa, Homo sapiens
ISWNSGSI

SEQ ID NO: 9 - protein, 15 aa, Homo sapiens
AKVSSQSGSVAGFDY

SEQ ID NO: 10 - protein, 8 aa, Homo sapiens
GGTFSSYA

SEQ ID NO: 11 - protein, 8 aa, Homo sapiens
IIPIFGTA

SEQ ID NO: 12 - protein, 10 aa, Homo sapiens
ARGATRGMDV

SEQ ID NO: 13 - protein, 8 aa, Homo sapiens
GFTFSSYA

SEQ ID NO: 14 - protein, 8 aa, Homo sapiens
ISYDGSNK

SEQ ID NO: 15 - protein, 16 aa, Homo sapiens
ARGGPYYDFWSDGMDV

SEQ ID NO: 16 - protein, 8 aa, Homo sapiens
GGTFSSYA

SEQ ID NO: 17 - protein, 8 aa, Homo sapiens
IIPILGIA

SEQ ID NO: 18 - protein, 14 aa, Homo sapiens
ARDRDTMVRGGMDV

SEQ ID NO: 19 - protein, 8 aa, Homo sapiens
GYTFSGRP

SEQ ID NO: 20 - protein, 8 aa, Homo sapiens
INPNNGGT

SEQ ID NO: 21 - protein, 12 aa, Homo sapiens
ASRPQYWGGLDV

SEQ ID NO: 22 - protein, 8 aa, Homo sapiens
GYTFSGRP

SEQ ID NO: 23 - protein, 8 aa, Homo sapiens
INPNNGGT

SEQ ID NO: 24 - protein, 12 aa, Homo sapiens
ASRPQYWGGLDV

SEQ ID NO: 25 - protein, 8 aa, Homo sapiens
GFTFSIYS

SEQ ID NO: 26 - protein, 8 aa, Homo sapiens
IGGGGTSI

SEQ ID NO: 27 - protein, 11 aa, Homo sapiens
ARGKPATGFDY

SEQ ID NO: 28 - protein, 8 aa, Homo sapiens
SSNIGSNT

SEQ ID NO: 29 - protein, 11 aa, Homo sapiens
AAWDDSLNGWV

SEQ ID NO: 30 - protein, 6 aa, Homo sapiens
NIGSRT

SEQ ID NO: 31 - protein, 11 aa, Homo sapiens
QVWDSSSDHLV

SEQ ID NO: 32 - protein, 8 aa, Homo sapiens
SSNIGANY

SEQ ID NO: 33 - protein, 11 aa, Homo sapiens
AGWDDSLSGVL

SEQ ID NO: 34 - protein, 6 aa, Homo sapiens
QSISNF

SEQ ID NO: 35 - protein, 9 aa, Homo sapiens
QQSSSIPLT

SEQ ID NO: 36 - protein, 8 aa, Homo sapiens
SGSIASNY

SEQ ID NO: 37 - protein, 9 aa, Homo sapiens
QSYDSSNYV

SEQ ID NO: 38 - protein, 9 aa, Homo sapiens
GSDIGGHNY

SEQ ID NO: 39 - protein, 9 aa, Homo sapiens
SSYAGSNVV

SEQ ID NO: 40 - protein, 6 aa, Homo sapiens
QGISSW

SEQ ID NO: 41 - protein, 10 aa, Homo sapiens
QQANSFLPLT

SEQ ID NO: 42 - protein, 6 aa, Homo sapiens
QGISSW

SEQ ID NO: 43 - protein, 10 aa, Homo sapiens
QQANSFLPLT

SEQ ID NO: 44 - protein, 9 aa, Homo sapiens
SSDVGGYNY

SEQ ID NO: 45 - protein, 11 aa, Homo sapiens
SSYAGGYTPYV

SEQ ID NO: 46 - protein, 120 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARFAFFSLASEDYWGQGTLVIVSS

SEQ ID NO: 47 - protein, 126 aa, Homo sapiens
EVQLVESGGGLVKPGGSLRLSCAASGFTFSWYTMNWVRQAPGKGLEWVSSISSSSSDMYYADSVKGRFTISRDNAKNSLYLQIDSLRAEDTAVYYCARAHYYDSTSLNHFYGMDVWGQGTTVTVSS

SEQ ID NO: 48 - protein, 122 aa, Homo sapiens
QVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKVSSQSGSVAGFDYWGQGTLVSVSS

SEQ ID NO: 49 - protein, 117 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGATRGMDVWGQGTLVTVSS

SEQ ID NO: 50 - protein, 123 aa, Homo sapiens
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARGGPYYDFWSDGMDVWGQGTMVTVSS

SEQ ID NO: 51 - protein, 121 aa, Homo sp.
QVQLMQSGAEVKKPGSSVKVSCKASGGTFSSYAISWVRQAPGQGLEWMGRIIPILGIANYAQKFQGRVTITADKSTSTAYMELSSLRSEDTAVYYCARDRDTMVRGGMDVWGQGTMVTVSS

SEQ ID NO: 52 - protein, 119 aa, Homo sapiens
QFRLVQSGAEVKKPGASVKVSCKASGYTFSGRPIHWVRQAPGQGLEWVGWINPNNGGTNYAQNFQGRVTMTRDTSITTAYMELSGLRSDDTAVYYCASRPQYWGGLDVWGQGTMVTVSS

SEQ ID NO: 53 - protein, 119 aa, Homo sapiens
QFHLMQSGAEVKKPGASVKVSCKASGYTFSGRPIHWVRQAPGQGLEWVGWINPNNGGTNYAQNFQGRVTMTRDTSITTAYMELSGLRSDDTAVYYCASRPQYWGGLDVWGQGTMITVSS

SEQ ID NO: 54 - protein, 118 aa, Homo sapiens
EVQLVQSGGGLAQPGGSLRLSCAASGFTFSIYSMNWVRQAPGKGLEWVSYIGGGGTSIYYADSVKGRFTISRDNAKNSLSLQMNSLRAEDTGVYYCARGKPATGFDYWGQGTLVSVSS

SEQ ID NO: 55 - protein, 110 aa, Homo sapiens
NFMLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGWVFGGGTQLTVL

SEQ ID NO: 56 - protein, 108 aa, Homo sapiens
QSVLTQPPSVSVAPGKTARITCGGNNIGSRTVHWYQQRPGQAPMLVVSDDNDRPSGIPERFSGSNSGNTATLTITRVEAGDEADYYCQVWDSSSDHLVFGGGTQLTVL

SEQ ID NO: 57 - protein, 110 aa, Homo sapiens
QPVLTQPPSASGTPGQRVTISCSGSSSNIGANYVYWYQQLPGTAPKLLIYRNNQRPSGVPDRFSGSKSGTSASLAIGGLRSEDEADYYCAGWDDSLSGVLFGGGTKVTVL

SEQ ID NO: 58 - protein, 107 aa, Homo sapiens
DIVMTQSPSSLSASVGDRVTITCRASQSISNFLNWYQQKPGKAPKLLIYAASSLQSGVPSRFSGSRSGTDFTLTISSLQPEDFATYYCQQSSSIPLTFGGGTKLEIK

SEQ ID NO: 59 - protein, 110 aa, Homo sapiens
QSVLTQPHSVSESPGKTVTISCTRSSGSIASNYVQWYQQRPGSSPTTVIYEDNQRPSGVPDRFSGSIDSSSNSASLTISGLKTEDEADYYCQSYDSSNYVFGTGTKVTVL

SEQ ID NO: 60 - protein, 109 aa, Homo sapiens
QSALNQPPSASGSPGQSVTISCTGTGSDIGGHNYVSWYQQHPGKAPKLMIYDVSKRPSRVPDRFSGSKSGNTASLTVSGLQAEDEADYYCSSYAGSNVVFGAGTKVTVL

SEQ ID NO: 61 - protein, 108 aa, Homo sapiens
DIQMTQSPSSVSASVGDRVTITCRASQGISSWLAWYQQKPGKAPKLLIYAASSLQSGVPSRFSGSGSGTDFTLTISSLQPEDFATYYCQQANSFLPLTFGGGTKVDIK

SEQ ID NO: 62 - protein, 108 aa, Homo sapiens
DIVMTQSPSSVSASVGDRVTITCRASQGISSWLAWYQQKPGKAPKLLIYAASSLQSGVPSRFSGSGSGTDFTLTISSLQPEDFATYYCQQANSFLPLTFGGGTKVDIK

SEQ ID NO: 63 - protein, 111 aa, Homo sapiens
QSVLTQPRSVSGSPGQSVTISCTGTSSDVGGYNYVSWYQQHPGKAPKLMISEVSKRPSGVPDRFSGSKSGNTASLTVSGLQAEDEADYYCSSYAGGYTPYVFGTGTKVTVL

SEQ ID NO: 64 - DNA, 24 bp, Homo sapiens
  source 1..24: mol_type = other DNA
ggaggcaccttcagcagctatgct

SEQ ID NO: 65 - DNA, 24 bp, Homo sapiens
  source 1..24: mol_type = other DNA
atcatccctatctttggtacagca

SEQ ID NO: 66 - DNA, 39 bp, Homo sapiens
  source 1..39: mol_type = other DNA
gcgaggttcgcctttttttctctagcctccgaggactac

SEQ ID NO: 67 - DNA, 24 bp, Homo sapiens
  source 1..24: mol_type = other DNA
ggattcaccttcagttggtacacc

SEQ ID NO: 68 - DNA, 24 bp, Homo sapiens
  source 1..24: mol_type = other DNA
attagtagtagtagtagtgacatg

SEQ ID NO: 69 - DNA, 57 bp, Homo sapiens
  source 1..57: mol_type = other DNA
gcgagagcccattattatgatagtactagtttaaaccacttctatggtatggacgtc

SEQ ID NO: 70 - DNA, 24 bp, Homo sapiens
  source 1..24: mol_type = other DNA
ggattcacctttgatgattatgcc

SEQ ID NO: 71 - DNA, 24 bp, Homo sapiens
  source 1..24: mol_type = other DNA
attagttggaatagtggtagcata

SEQ ID NO: 72 - DNA, 45 bp, Homo sapiens
  source 1..45: mol_type = other DNA
gcaaaagtttcttcgcaatcggggtcagtggctggttttgactac

SEQ ID NO: 73 - DNA, 24 bp, Homo sapiens
  source 1..24: mol_type = other DNA
ggaggcaccttcagcagctatgct

SEQ ID NO: 74 - DNA, 24 bp, Homo sapiens
  source 1..24: mol_type = other DNA
atcatccctatctttggtacagca

SEQ ID NO: 75 - DNA, 30 bp, Homo sapiens
  source 1..30: mol_type = other DNA
gcgagaggagctacccggggtatggacgtc

SEQ ID NO: 76 - DNA, 24 bp, Homo sapiens
  source 1..24: mol_type = other DNA
ggattcaccttcagtagctatgct

SEQ ID NO: 77 - DNA, 24 bp, Homo sapiens
  source 1..24: mol_type = other DNA
atatcatatgatggaagcaataaa

SEQ ID NO: 78 - DNA, 48 bp, Homo sapiens
  source 1..48: mol_type = other DNA
gcgagagggggcccgtattacgatttttggagtgacggtatggacgtc

SEQ ID NO: 79 - DNA, 24 bp, Homo sapiens
  source 1..24: mol_type = other DNA
ggaggcaccttcagcagctatgct

SEQ ID NO: 80 - DNA, 24 bp, Homo sapiens
  source 1..24: mol_type = other DNA
atcatccctatccttggtatagca

SEQ ID NO: 81 - DNA, 42 bp, Homo sapiens
  source 1..42: mol_type = other DNA
gcgagagatcgggatactatggttcgggggggcatggacgtc

SEQ ID NO: 82 - DNA, 24 bp, Homo sapiens
  source 1..24: mol_type = other DNA
ggatacaccttcagcggccgccct

SEQ ID NO: 83 - DNA, 24 bp, Homo sapiens
  source 1..24: mol_type = other DNA
atcaaccctaacaatggtggcaca

SEQ ID NO: 84 - DNA, 36 bp, Homo sapiens
  source 1..36: mol_type = other DNA
gcgagtcgacctcaatattgggggggtttggacgtc

SEQ ID NO: 85 - DNA, 24 bp, Homo sapiens
  source 1..24: mol_type = other DNA
ggatacaccttcagcggccgccct

SEQ ID NO: 86 - DNA, 24 bp, Homo sapiens
  source 1..24: mol_type = other DNA
atcaaccctaacaatggtggcaca

SEQ ID NO: 87 - DNA, 36 bp, Homo sapiens; identical to SEQ ID NO: 84
  source 1..36: mol_type = other DNA

SEQ ID NO: 88 - DNA, 24 bp, Homo sapiens
  source 1..24: mol_type = other DNA
ggattcaccttcagtatctatagc

SEQ ID NO: 89 - DNA, 24 bp, Homo sapiens
  source 1..24: mol_type = other DNA
attggcggtggtggtacttccata

SEQ ID NO: 90 - DNA, 33 bp, Homo sapiens
  source 1..33: mol_type = other DNA
gcgagagggaagcctgctaccggatttgactac

SEQ ID NO: 91 - DNA, 24 bp, Homo sapiens
  source 1..24: mol_type = other DNA
agctccaacatcggaagtaatact

SEQ ID NO: 92 - DNA, 33 bp, Homo sapiens
  source 1..33: mol_type = other DNA
gcagcatgggatgacagcctgaatggttgggtg

SEQ ID NO: 93 - DNA, 18 bp, Homo sapiens
  source 1..18: mol_type = other DNA
aacattggaagtagaact

SEQ ID NO: 94 - DNA, 33 bp, Homo sapiens
  source 1..33: mol_type = other DNA
caggtgtgggatagtagtagtgatcatctggtg

SEQ ID NO: 95 - DNA, 24 bp, Homo sapiens
  source 1..24: mol_type = other DNA
agctccaacatcggagctaattat

SEQ ID NO: 96 - DNA, 33 bp, Homo sapiens
  source 1..33: mol_type = other DNA
gcaggatgggatgacagtctgagtggtgtacta

SEQ ID NO: 97 - DNA, 18 bp, Homo sapiens
  source 1..18: mol_type = other DNA
cagagcattagcaacttt

SEQ ID NO: 98 - DNA, 27 bp, Homo sapiens
  source 1..27: mol_type = other DNA
caacagagttccagtataccactcact

SEQ ID NO: 99 - DNA, 24 bp, Homo sapiens
  source 1..24: mol_type = other DNA
agtggcagcattgccagcaactat

SEQ ID NO: 100 - DNA, 27 bp, Homo sapiens
  source 1..27: mol_type = other DNA
cagtcttatgatagcagcaattatgtc

SEQ ID NO: 101 - DNA, 27 bp, Homo sapiens
  source 1..27: mol_type = other DNA
ggcagtgacattggtggtcataactat

SEQ ID NO: 102 - DNA, 27 bp, Homo sapiens
  source 1..27: mol_type = other DNA
agctcgtatgcaggcagtaatgtggta

SEQ ID NO: 103 - DNA, 30 bp, Homo sapiens
  source 1..30: mol_type = other DNA
caacaggctaacagtttccttcctctcact

SEQ ID NO: 104 - DNA, 18 bp, Homo sapiens
  source 1..18: mol_type = other DNA
cagggtattagcagctgg

SEQ ID NO: 105 - DNA, 30 bp, Homo sapiens; identical to SEQ ID NO: 103
  source 1..30: mol_type = other DNA

SEQ ID NO: 106 - DNA, 27 bp, Homo sapiens
  source 1..27: mol_type = other DNA
agcagtgatgttggtggttataactat

SEQ ID NO: 107 - DNA, 33 bp, Homo sapiens
  source 1..33: mol_type = other DNA
tcctcatatgcaggcgggtacaccccctatgtc

SEQ ID NO: 108 - DNA, 360 bp, Homo sapiens
  source 1..360: mol_type = other DNA
caggtgcagctggtgcaatctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgaggttcgcctttttttctctagcctccgaggactactggggccagggaaccctggtcatcgtctcctca

SEQ ID NO: 109 - DNA, 378 bp, Homo sapiens
  source 1..378: mol_type = other DNA
gaagtgcagctggtggagtctgggggaggcctggtcaagcctggggggtccctgagactctcttgtgcagcctctggattcaccttcagttggtacaccatgaactgggtccgccaggctccagggaaggggctggagtgggtctcatccattagtagtagtagtagtgacatgtattatgcagactcagtgaagggccgattcaccatctccagagacaacgccaagaactcactttatctgcaaatagacagcctgagagccgaggacacggctgtgtattattgtgcgagagcccattattatgatagtactagtttaaaccacttctatggtatggacgtctggggccaggggaccacggtcaccgtctcctca

SEQ ID NO: 110 - DNA, 366 bp, Homo sapiens
  source 1..366: mol_type = other DNA
caggtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaagtttcttcgcaatcggggtcagtggctggttttgactactggggccagggaaccctggtcagcgtctcctca

SEQ ID NO: 111 - DNA, 351 bp, Homo sapiens
  source 1..351: mol_type = other DNA
caggtgcagctggtgcaatctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagaggagctacccggggtatggacgtctggggccaaggcaccctggtcaccgtctcttca

SEQ ID NO: 112 - DNA, 369 bp, Homo sapiens
  source 1..369: mol_type = other DNA
caggtgcagctggtggagtctgggggaggcgtggtccagcctgggaggtccctgagactctcctgtgcagcctctggattcaccttcagtagctatgctatgcactgggtccgccaggctccaggcaaggggctggagtgggtggcagttatatcatatgatggaagcaataaatactacgcagactccgtgaagggccgattcaccatctccagagacaattccaagaacacgctgtatctgcaaatgaacagcctgagagctgaggacacggctgtgtattactgtgcgagagggggcccgtattacgatttttggagtgacggtatggacgtctggggccaagggacaatggtcaccgtctcctca

SEQ ID NO: 113 - DNA, 363 bp, Homo sapiens
  source 1..363: mol_type = other DNA
caggtgcagttgatgcagtctggggctgaggtgaagaagcctgggtcctcggtgaaggtctcctgcaaggcttctggaggcaccttcagcagctatgctatcagctgggtgcgacaggcccctggacaagggcttgagtggatgggaaggatcatccctatccttggtatagcaaactacgcacagaagttccagggcagagtcacgattaccgcggacaaatccacgagcacagcctacatggagctgagcagcctgagatctgaggacacggccgtgtattactgtgcgagagatcgggatactatggttcgggggggcatggacgtctggggccaagggacaatggtcaccgtctcttca

SEQ ID NO: 114 - DNA, 420 bp, Homo sapiens
  source 1..420: mol_type = other DNA
cagtttcgcctggtgcagtctggggctgaggtgaagaagcctggggcctcagtgaaggtctcctgcaaggcttctggatacaccttcagcggccgccctatacactgggtgcgacaggcccccggacaagggcttgagtgggtgggatggatcaaccctaacaatggtggcacaaattatgcgcagaattttcagggcagggtcaccatgaccagggacacgtccatcaccacagcctacatggaactgagcggactgagatctgacgacacggccgtatattactgtgcgagtcgacctcaatattgggggggtttggacgtctggggccaagggacaatggtcaccgtctcttcagatactatggttcgggggggcatggacgtctggggccaagggacaatggtcaccgtctcttca

SEQ ID NO: 115 - DNA, 357 bp, Homo sapiens
  source 1..357: mol_type = other DNA
cagtttcacctgatgcagtctggggctgaggtgaagaagcctggggcctcagtgaaggtctcctgcaaggcttctggatacaccttcagcggccgccctatacactgggtgcgacaggcccccggacaagggcttgagtgggtgggatggatcaaccctaacaatggtggcacaaattatgcgcagaattttcagggcagggtcaccatgaccagggacacgtccatcaccacagcctacatggaactgagcggactgagatctgacgacacggccgtatattactgtgcgagtcgacctcaatattgggggggtttggacgtctggggccaagggacaatgatcacggtctcttcc

SEQ ID NO: 116 - DNA, 354 bp, Homo sapiens
  source 1..354: mol_type = other DNA
gaggtgcagctggtgcagtctgggggaggcttggcacagcctggggggtccctgagactctcctgtgcagcctctggattcaccttcagtatctatagcatgaattgggtccgccaggctccagggaagggactggagtgggtttcatacattggcggtggtggtacttccatatactacgcagactctgtgaagggccgattcaccatctccagagacaatgccaagaactcactgtctctgcaaatgaacagcctgagagccgaggacacgggtgtgtattactgtgcgagagggaagcctgctaccggatttgactactggggccagggaaccctggtcagcgtctcctca

SEQ ID NO: 117 - DNA, 330 bp, Homo sapiens
  source 1..330: mol_type = other DNA
aattttatgctgactcagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggttgggtgttcggcggagggacccagctgaccgtccta

SEQ ID NO: 118 - DNA, 18 bp, Homo sapiens
  source 1..18: mol_type = other DNA
cagggtattagcagctgg

SEQ ID NO: 119 - DNA, 330 bp, Homo sapiens
  source 1..330: mol_type = other DNA
cagcctgtgctgactcagccaccctcagcgtctgggacccccgggcagagggtcaccatttcttgttctggaagcagctccaacatcggagctaattatgtctactggtaccagcagctcccaggaacggcccccaaactcctcatctataggaataatcagcggccctcaggggtccctgaccgattctctggctccaagtctggcacctcagcctccctggccatcggtgggctccggtccgaggatgaggctgattattactgtgcaggatgggatgacagtctgagtggtgtactattcggcggagggaccaaggtcaccgtccta

SEQ ID NO: 120 - DNA, 321 bp, Homo sapiens
  source 1..321: mol_type = other DNA
gacatcgtgatgacccagtctccatcctccctgtctgcatctgtaggagacagagtcaccatcacttgccgggcaagtcagagcattagcaactttttaaattggtatcagcagaaaccagggaaagcccctaagctcctgatctatgctgcatccagtttgcaaagtggggtcccatcaaggttcagtggcagtcgatctgggacagatttcactctcaccatcagcagtctgcaacctgaagattttgcaacttactactgtcaacagagttccagtataccactcactttcggcggagggaccaagctggagatcaaa

SEQ ID NO: 121 - DNA, 330 bp, Homo sapiens
  source 1..330: mol_type = other DNA
cagtctgtgctgactcagccccactctgtgtcggagtctccggggaagacggtaaccatctcctgcacccgcagcagtggcagcattgccagcaactatgtgcagtggtaccagcagcgcccgggcagttcccccaccactgtgatctatgaggataaccaaagaccctctggggtccctgatcggttctctggctccatcgacagctcctccaactctgcctccctcaccatctctggactgaagactgaggacgaggctgactactactgtcagtcttatgatagcagcaattatgtcttcggaactgggaccaaggtcaccgtccta

SEQ ID NO: 122 - DNA, 327 bp, Homo sapiens
  source 1..327: mol_type = other DNA
cagtctgccctgaatcagcctccctccgcgtccgggtctcctggacagtcagtcaccatctcctgcactggaaccggcagtgacattggtggtcataactatgtctcctggtaccaacagcacccaggcaaggcccccaaactcatgatttatgacgtcagtaagcggccctcacgggtccctgatcgcttctctggctccaagtctggcaacacggcctccctgaccgtctctgggctccaggctgaggatgaggcagattattactgcagctcgtatgcaggcagtaatgtggtattcggcgcagggaccaaggtcaccgtccta

SEQ ID NO: 123 - DNA, 324 bp, Homo sapiens
  source 1..324: mol_type = other DNA
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaacaggctaacagtttccttcctctcactttcggcggagggaccaaagtggatatcaaa

SEQ ID NO: 124 - DNA, 324 bp, Homo sapiens
  source 1..324: mol_type = other DNA
gacatcgtgatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaacaggctaacagtttccttcctctcactttcggcggagggaccaaagtggatatcaaa

SEQ ID NO: 125 - DNA, 333 bp, Homo sapiens
  source 1..333: mol_type = other DNA
cagtctgtgctgactcagcctcgctcagtgtccgggtctcctggacagtcagtcaccatctcctgcactggaaccagcagtgatgttggtggttataactatgtctcctggtaccaacaacacccaggcaaagcccccaaactcatgatttctgaggtcagtaagcggccctcaggggtccctgatcgcttctctggctccaagtctggcaacacggcctccctgaccgtctctgggctccaggctgaggatgaggctgattattactgctcctcatatgcaggcgggtacaccccctatgtcttcggaactgggaccaaggtcaccgtccta

SEQ ID NO: 126 - protein, 8 aa, Homo sapiens
GYSFTSYW

SEQ ID NO: 127 - protein, 8 aa, Homo sp.
IDPSDSYT

SEQ ID NO: 128 - protein, 18 aa, Homo sapiens
ARHPLAYCGGDCQRGFDY

SEQ ID NO: 129 - protein, 8 aa, Homo sapiens
GGTFSSYA

SEQ ID NO: 130 - protein, 8 aa, Homo sapiens
IIPIFGTA

SEQ ID NO: 131 - protein, 12 aa, Homo sapiens
ARSLGWYYAFDI

SEQ ID NO: 132 - protein, 8 aa, Homo sapiens
GYTFTSYG

SEQ ID NO: 133 - protein, 8 aa, Homo sapiens
ISAYNGNT

SEQ ID NO: 134 - protein, 12 aa, Homo sapiens
ARSLGWYYAFDI

SEQ ID NO: 135 - protein, 8 aa, Homo sapiens
GYSFTSYW

SEQ ID NO: 136 - protein, 8 aa, Homo sapiens
ISAYNGNT

SEQ ID NO: 137 - protein, 16 aa, Homo sapiens
ARHPPRSTRGDYGMDV

SEQ ID NO: 138 - protein, 8 aa, Homo sapiens
GFSVSSYY

SEQ ID NO: 139 - protein, 7 aa, Homo sapiens
TYTDGST

SEQ ID NO: 140 - protein, 14 aa, Homo sapiens
AREGDGYNSGGLDY

SEQ ID NO: 141 - protein, 8 aa, Homo sapiens
GGTFSSYA

SEQ ID NO: 142 - protein, 8 aa, Homo sapiens
IIPIFGTA

SEQ ID NO: 143 - protein, 13 aa, Homo sapiens
AVSIFGVVIPLDY

SEQ ID NO: 144 - protein, 8 aa, Homo sapiens
GGTFSSYA

SEQ ID NO: 145 - protein, 8 aa, Homo sapiens
IIPIFGTA

SEQ ID NO: 146 - protein, 12 aa, Homo sapiens
ARSGYYNDAFDI

SEQ ID NO: 147 - protein, 8 aa, Homo sapiens
GYTFTSYG

SEQ ID NO: 148 - protein, 8 aa, Homo sapiens
ISAYNGNT

SEQ ID NO: 149 - protein, 17 aa, Homo sapiens
ARDQKLLHILYYYGMDV

SEQ ID NO: 150 - protein, 8 aa, Homo sapiens
GYSFTSYW

SEQ ID NO: 151 - protein, 8 aa, Homo sapiens
IDPSDSYT

SEQ ID NO: 152 - protein, 12 aa, Homo sapiens
ARARTWFDAFDI

SEQ ID NO: 153 - protein, 8 aa, Homo sapiens
GYTFTSYG

SEQ ID NO: 154 - protein, 8 aa, Homo sapiens
ISAYNGNT

SEQ ID NO: 155 - protein, 12 aa, Homo sapiens
ARVGYYYYGMDV

SEQ ID NO: 156 - protein, 6 aa, Homo sapiens
KLREDL

SEQ ID NO: 157 - protein, 10 aa, Homo sapiens
QAWDNSVDYV

SEQ ID NO: 158 - protein, 6 aa, Homo sapiens
QNINNW

SEQ ID NO: 159 - protein, 13 aa, Homo sapiens
QQYKNYPYSNLYT

SEQ ID NO: 160 - protein, 6 aa, Homo sapiens
QSISSY

SEQ ID NO: 161 - protein, 9 aa, Homo sapiens
QQSYSTPPT

SEQ ID NO: 162 - protein, 8 aa, Homo sapiens
NSNIGSYI

SEQ ID NO: 163 - protein, 11 aa, Homo sapiens
AAWDNILNNYV

SEQ ID NO: 164 - protein, 6 aa, Homo sapiens
QSIGTY

SEQ ID NO: 165 - protein, 9 aa, Homo sapiens
QQTYITPRT

SEQ ID NO: 166 - protein, 6 aa, Homo sapiens
NIGRKS

SEQ ID NO: 167 - protein, 11 aa, Homo sapiens
QVWDSSSASGV

SEQ ID NO: 168 - protein, 9 aa, Homo sapiens
NSDIGGYNY

SEQ ID NO: 169 - protein, 10 aa, Homo sapiens
SSYTSSSTLV

SEQ ID NO: 170 - protein, 11 aa, Homo sapiens
QSLLHSNGNNY

SEQ ID NO: 171 - protein, 9 aa, Homo sapiens
MQALQTAYT

SEQ ID NO: 172 - protein, 8 aa, Homo sapiens
SSNIGRYS

SEQ ID NO: 173 - protein, 11 aa, Homo sp.
AAWDDSLKGWV

SEQ ID NO: 174 - protein, 9 aa, Homo sapiens
SSNIGAGFD

SEQ ID NO: 175 - protein, 11 aa, Homo sapiens
QSYDRSLSGYV

SEQ ID NO: 176 - protein, 125 aa, Homo sapiens
QVQLVQSGAEVKKPGESLRISCKGSGYSFTSYWISWVRQMPGKGLEWMGRIDPSDSYTNYSPSFQGHVTISADKSISTAYLQWSSLKASDTAMYYCARHPLAYCGGDCQRGFDYWGQGTLVTVSS

SEQ ID NO: 177 - protein, 119 aa, Homo sapiens
QVQLVQSGAEVKKPGSSMKVSCKASGGTFSSYAISWVRQAPGQGLEWMGGIIPIFGTAKYAQKFQGRVTITADESTTTAYMELSSLSSEDTAVYYCARSLGWYYAFDIWGQGTMVTVSS

SEQ ID NO: 178 - protein, 125 aa, Homo sapiens
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDSRMSLEYAPGYGMDVWGQGTTVTVSS

SEQ ID NO: 179 - protein, 123 aa, Homo sapiens
QVQLVQSGAEVKKPGESLRISCKGSGYSFTSYWISWVRQMPGKGLEWMGRIDPSDSYTNYSPSFQGHVTISADKSISTAYLQWSSLKASDTAMYYCARHPPRSTRGDYGMDVWGQGTMVTVSS

SEQ ID NO: 180 - protein, 120 aa, Homo sapiens
EVQLLESGGGLIQPGGSLRLSCAASGFSVSSYYMNWVRQAPGKGLEWVSNTYTDGSTSYADSVGGRFSVSRDNSKNTLHLQMNSLRVEDTAVYYCAREGDGYNSGGLDYWGQGTLVTVSS

SEQ ID NO: 181 - protein, 120 aa, Homo sapiens
PVLLVQSGAEVKKPGSSVKVSCKASGGTFSSYAISWVRQAPGQGLEWMGGIIPIFGTANYAQKFQGRVTITADESTSTAYMELSSLRSEDTAVYYCAVSIFGVVIPLDYWGQGTLVIVSS

SEQ ID NO: 182 - protein, 119 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARSGYYNDAFDIWGQGTMVTVSS

SEQ ID NO: 183 - protein, 124 aa, Homo sapiens
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDQKLLHILYYYGMDVWGQGTTVIVSS

SEQ ID NO: 184 - protein, 119 aa, Homo sapiens
QVQLVQSGAEVKKPGESLRISCKGSGYSFTSYWISWVRQMPGKGLEWMGRIDPSDSYTNYSPSFQGHVTISADKSISTAYLQWSSLKASDTAIYYCARARTWFDAFDIWGQGTTVTVSS

SEQ ID NO: 185 - protein, 119 aa, Homo sapiens
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARVGYYYYGMDVWGQGTTVTVSS

SEQ ID NO: 186 - protein, 107 aa, Homo sapiens
QPVLTQPPSVSVSPGQTARITCSGDKLREDLTSWYQQKPGQSPVLVIYQDSKRPSGIPARFSGSISGSTATLTISGTQAVDEADYYCQAWDNSVDYVFGTGTKVTVL

SEQ ID NO: 187 - protein, 111 aa, Homo sapiens
DIVMTQSPSTLSASVGDRVTITCRASQNINNWLAWYQQKPGKAPKLLIYKASTAESGVPSRFSGSGFGTEFTLTISSLQPDDFASYYCQQYKNYPYSNLYTFGQGTKVDIK

SEQ ID NO: 188 - protein, 107 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGGGTKVDIK

SEQ ID NO: 189 - protein, 110 aa, Homo sapiens
QAVVTQPPSVSGTPGQRVTISCSGSNSNIGSYIVNWYQQLPGTAPKLLIWGNDRRPSGVPDRVSASKSGTSASLAISGLQSEDEGDYYCAAWDNILNNYVFGPGTQLTVL

SEQ ID NO: 190 - protein, 107 aa, Homo sapiens
DIQLTQSPASLSASLGDRVTITCRASQSIGTYLNWYQQRPGQAPKLLIFGTSSLQSGVPSRFSGSGSGTDFTLTISSLQPEDLATYYCQQTYITPRTFGQGTKVEIK

SEQ ID NO: 191 - protein, 108 aa, Homo sapiens
QPVLTQSPSVSLAPGQTAKITCGGTNIGRKSVHWYQQKPGQAPVMVIYYDSDRPSGIPERFAGSNSGNTATLTISRVEAGDEAVYYCQVWDSSSASGVFGGGTKVTVL

SEQ ID NO: 192 - protein, 110 aa, Homo sapiens
QSALTQPASVSGSPGQSITISCTGTNSDIGGYNYVSWYQQHPGKAPKLMIYEVSNRPSGVSNRFSGSKSGNTASLTISGLQAEDEAHYYCSSYTSSSTLVFGGGTKVTVL

SEQ ID NO: 193 - protein, 112 aa, Homo sapiens
DIVMTQSPLSLSVTPGEPASISCRSSQSLLHSNGNNYLDWYLQKPGQSPQLLIYLGSNRASGVPDRFSGSGSGTDFSLKINKVEAEDVGVYYCMQALQTAYTFGQGTKLEIK

SEQ ID NO: 194 - protein, 110 aa, Homo sapiens
QAVVTQPPSASGTPGQRVTISCSGGSSNIGRYSVNWYQQLPGTAPKLLINSNIQRPSGVPDRFSGSKSGTSASLAISGLQSEDEADYHCAAWDDSLKGWVFGGGTKVTVL

SEQ ID NO: 195 - protein, 111 aa, Homo sapiens
QSVLTQPPSVSGAPGQRVTISCTGSSSNIGAGFDVHWYQQLPGTAPKLLIYANSNRPSGVPDRFSASKSGTSASLAITGLQAEDEADYYCQSYDRSLSGYVFGSGTKVTVL

SEQ ID NO: 196 - protein, 25 aa, Homo sapiens
QVQLVQSGAEVKKPGESLRISCKGS

SEQ ID NO: 197 - protein, 17 aa, Homo sapiens
ISWVRQMPGKGLEWMGR

SEQ ID NO: 198 - protein, 38 aa, Homo sapiens
NYSPSFQGHVTISADKSISTAYLQWSSLKASDTAMYYC

SEQ ID NO: 199 - protein, 11 aa, Homo sapiens
WGQGTLVTVSS

SEQ ID NO: 200 - protein, 25 aa, Homo sapiens
QVQLVQSGAEVKKPGSSMKVSCKAS

SEQ ID NO: 201 - protein, 17 aa, Homo sapiens
ISWVRQAPGQGLEWMGG

SEQ ID NO: 202 - protein, 38 aa, Homo sapiens; identical to SEQ ID NO: 198

SEQ ID NO: 203 - protein, 11 aa, Homo sapiens
WGQGTMVTVSS

SEQ ID NO: 204 - protein, 25 aa, Homo sapiens [sequence omitted]

SEQ ID NO: 205 - protein, 17 aa, Homo sapiens
ISWVRQAPGQGLEWMGW

SEQ ID NO: 206 - protein, 38 aa, Homo sapiens [sequence omitted]

SEQ ID NO: 207 - protein, 11 aa, Homo sapiens
WGQGTTVTVSS

SEQ ID NO: 208 - protein, 25 aa, Homo sapiens
QVQLVQSGAEVKKPGESLRISCKGS

SEQ ID NO: 209 - protein, 17 aa, Homo sapiens
ISWVRQMPGKGLEWMGR

SEQ ID NO: 210 - protein, 38 aa, Homo sapiens; identical to SEQ ID NO: 198

SEQ ID NO: 211 - protein, 11 aa, Homo sapiens
WGQGTMVTVSS

SEQ ID NO: 212 - protein, 25 aa, Homo sapiens
EVQLLESGGGLIQPGGSLRLSCAAS

SEQ ID NO: 213 - protein, 17 aa, Homo sapiens
MNWVRQAPGKGLEWVSN

SEQ ID NO: 214 - protein, 38 aa, Homo sapiens
SYADSVGGRFSVSRDNSKNTLHLQMNSLRVEDTAVYYC

SEQ ID NO: 215 - protein, 11 aa, Homo sapiens
WGQGTMVTVSS

SEQ ID NO: 216 - protein, 25 aa, Homo sapiens
PVLLVQSGAEVKKPGSSVKVSCKAS

SEQ ID NO: 217 - protein, 17 aa, Homo sapiens
ISWVRQAPGQGLEWMGG

SEQ ID NO: 218 - protein, 38 aa, Homo sapiens; identical to SEQ ID NO: 214

SEQ ID NO: 219 - protein, 11 aa, Homo sapiens
WGQGTLVIVSS

SEQ ID NO: 220 - protein, 25 aa, Homo sapiens [sequence omitted]

SEQ ID NO: 221 - protein, 17 aa, Homo sapiens
ISWVRQAPGQGLEWMGG

SEQ ID NO: 222 - protein, 38 aa, Homo sapiens
NYAQKFQGRVTITADESTSTAYMELSSLRSEDTAVYYC

SEQ ID NO: 223 - protein, 11 aa, Homo sapiens
WGQGTMVTVSS

SEQ ID NO: 224 - protein, 25 aa, Homo sapiens [sequence omitted]

SEQ ID NO: 225 - protein, 17 aa, Homo sapiens
ISWVRQAPGQGLEWMGW

SEQ ID NO: 226 - protein, 38 aa, Homo sapiens [sequence omitted]

SEQ ID NO: 227 - protein, 11 aa, Homo sapiens
WGQGTTVIVSS

SEQ ID NO: 228 - protein, 25 aa, Homo sapiens
QVQLVQSGAEVKKPGESLRISCKGS

SEQ ID NO: 229 - protein, 17 aa, Homo sapiens
ISWVRQMPGKGLEWMGR

SEQ ID NO: 230 - protein, 38 aa, Homo sapiens
NYSPSFQGHVTISADKSISTAYLQWSSLKASDTAIYYC

SEQ ID NO: 231 - protein, 11 aa, Homo sapiens
WGQGTTVTVSS

SEQ ID NO: 232 - protein, 25 aa, Homo sapiens [sequence omitted]

SEQ ID NO: 233 - protein, 17 aa, Homo sapiens
ISWVRQAPGQGLEWMGW

SEQ ID NO: 234 - protein, 38 aa, Homo sapiens [sequence omitted]

SEQ ID NO: 235 - protein, 11 aa, Homo sapiens
WGQGTTVTVSS

SEQ ID NO: 236 - DNA, 324 bp, Homo sapiens
  source 1..324: mol_type = other DNA
cagtctgtgctgactcagccaccctcggtgtcagtggccccaggaaagacggccaggattacctgtgggggaaacaacattggaagtagaactgtacactggtaccagcagaggccaggccaggcccctatgttggtcgtctctgatgataacgaccggccctcagggatccctgagcgattctctggctccaactctgggaacacggccaccctgaccatcaccagggtcgaagccggggatgaggccgactattactgtcaggtgtgggatagtagtagtgatcatctggtgttcggcggagggacccagctgaccgtccta

SEQ ID NO: 237 - protein, 25 aa, Homo sapiens
QPVLTQPPSVSVSPGQTARITCSGD

SEQ ID NO: 238 - protein, 17 aa, Homo sapiens
TSWYQQKPGQSPVLVIY

SEQ ID NO: 239 - protein, 36 aa, Homo sapiens
KRPSGIPARFSGSISGSTATLTISGTQAVDEADYYC

SEQ ID NO: 240 - protein, 10 aa, Homo sapiens
FGTGTKVTVL

SEQ ID NO: 241 - protein, 26 aa, Homo sapiens
DIVMTQSPSTLSASVGDRVTITCRAS

SEQ ID NO: 242 - protein, 17 aa, Homo sapiens
LAWYQQKPGKAPKLLIY

SEQ ID NO: 243 - protein, 36 aa, Homo sapiens
TAESGVPSRFSGSGFGTEFTLTISSLQPDDFASYYC

SEQ ID NO: 244 - protein, 10 aa, Homo sapiens
FGQGTKVDIK

SEQ ID NO: 245 - protein, 26 aa, Homo sapiens [sequence omitted]

SEQ ID NO: 246 - protein, 17 aa, Homo sapiens
LNWYQQKPGKAPKLLIY

SEQ ID NO: 247 - protein, 36 aa, Homo sapiens
SLQSGVPSRFSGSGSGTDFTLTISSLQPEDFATYYC

SEQ ID NO: 248 - protein, 10 aa, Homo sapiens
FGGGTKVDIK

SEQ ID NO: 249 - protein, 25 aa, Homo sapiens
QAVVTQPPSVSGTPGQRVTISCSGS

SEQ ID NO: 250 - protein, 17 aa, Homo sapiens
VNWYQQLPGTAPKLLIW

SEQ ID NO: 251 - protein, 36 aa, Homo sapiens
RRPSGVPDRVSASKSGTSASLAISGLQSEDEGDYYC

SEQ ID NO: 252 - protein, 10 aa, Homo sapiens
FGPGTQLTVL

SEQ ID NO: 253 - protein, 26 aa, Homo sapiens
DIQLTQSPASLSASLGDRVTITCRAS

SEQ ID NO: 254 - protein, 17 aa, Homo sapiens
LNWYQQRPGQAPKLLIF

SEQ ID NO: 255 - protein, 36 aa, Homo sapiens
SLQSGVPSRFSGSGSGTDFTLTISSLQPEDLATYYC

SEQ ID NO: 256 - protein, 10 aa, Homo sapiens
FGQGTKVEIK

SEQ ID NO: 257 - protein, 25 aa, Homo sapiens
QPVLTQSPSVSLAPGQTAKITCGGT

SEQ ID NO: 258 - protein, 17 aa, Homo sapiens
VHWYQQKPGQAPVMVIY

SEQ ID NO: 259 - protein, 36 aa, Homo sapiens
DRPSGIPERFAGSNSGNTATLTISRVEAGDEAVYYC

SEQ ID NO: 260 - protein, 10 aa, Homo sapiens
FGGGTKVTVL

SEQ ID NO: 261 - protein, 25 aa, Homo sapiens
QSALTQPASVSGSPGQSITISCTGT

SEQ ID NO: 262 - protein, 17 aa, Homo sapiens
VSWYQQHPGKAPKLMIY

SEQ ID NO: 263 - protein, 36 aa, Homo sapiens
NRPSGVSNRFSGSKSGNTASLTISGLQAEDEAHYYC

SEQ ID NO: 264 - protein, 10 aa, Homo sapiens
FGGGTKVTVL

SEQ ID NO: 265 - protein, 26 aa, Homo sapiens
DIVMTQSPLSLSVTPGEPASISCRSS

SEQ ID NO: 266 - protein, 17 aa, Homo sapiens
LDWYLQKPGQSPQLLIY

SEQ ID NO: 267 - protein, 36 aa, Homo sapiens
NRASGVPDRFSGSGSGTDFSLKINKVEAEDVGVYYC

SEQ ID NO: 268 - protein, 10 aa, Homo sapiens
FGQGTKLEIK

SEQ ID NO: 269 - protein, 25 aa, Homo sapiens
QAVVTQPPSASGTPGQRVTISCSGG

SEQ ID NO: 270 - protein, 17 aa, Homo sapiens
VNWYQQLPGTAPKLLIN

SEQ ID NO: 271 - protein, 36 aa, Homo sapiens
QRPSGVPDRFSGSKSGTSASLAISGLQSEDEADYHC

SEQ ID NO: 272 - protein, 10 aa, Homo sapiens
FGGGTKVTVL

SEQ ID NO: 273 - protein, 25 aa, Homo sapiens
QSVLTQPPSVSGAPGQRVTISCTGS

SEQ ID NO: 274 - protein, 17 aa, Homo sapiens
VHWYQQLPGTAPKLLIY

SEQ ID NO: 275 - protein, 36 aa, Homo sapiens
NRPSGVPDRFSASKSGTSASLAITGLQAEDEADYYC

SEQ ID NO: 276 - protein, 10 aa, Homo sapiens
FGSGTKVTVL

SEQ ID NO: 277 - DNA, 24 bp, Homo sapiens
  source 1..24: mol_type = other DNA
ggatacagctttaccagctactgg

SEQ ID NO: 278 - DNA, 24 bp, Homo sapiens
  source 1..24: mol_type = other DNA
attgatcctagtgactcttatacc

SEQ ID NO: 279 - DNA, 54 bp, Homo sapiens
  source 1..54: mol_type = other DNA
gcgagacatcccctagcatattgtggtggtgactgccaacggggctttgactac

SEQ ID NO: 280 - DNA, 24 bp, Homo sapiens
  source 1..24: mol_type = other DNA
ggaggcaccttcagcagttatgct

SEQ ID NO: 281 - DNA, 24 bp, Homo sapiens
  source 1..24: mol_type = other DNA
atcatccctatctttggtacagca

SEQ ID NO: 282 - DNA, 36 bp, Homo sapiens
  source 1..36: mol_type = other DNA
gcgagaagtcttggctggtactatgcttttgatatc

SEQ ID NO: 283 - DNA, 24 bp, Homo sapiens
  source 1..24: mol_type = other DNA
ggttacacctttaccagctatggt

SEQ ID NO: 284 - DNA, 24 bp, Homo sapiens
  source 1..24: mol_type = other DNA
atcagcgcttacaatggtaacaca

SEQ ID NO: 285 - DNA, 54 bp, Homo sapiens
  source 1..54: mol_type = other DNA
gcgagagatagtagaatgtcactggagtatgctcccgggtacggaatggacgtc

SEQ ID NO: 286 - DNA, 24 bp, Homo sapiens
  source 1..24: mol_type = other DNA
ggatacagctttaccagctactgg

SEQ ID NO: 287 - DNA, 24 bp, Homo sapiens
  source 1..24: mol_type = other DNA
attgatcctagtgactcttatacc

SEQ ID NO: 288 - DNA, 48 bp, Homo sapiens
  source 1..48: mol_type = other DNA
gcgagacatcccccccgatcaacgcggggtgactacggtatggacgtc

SEQ ID NO: 289 - DNA, 24 bp, Homo sapiens
  source 1..24: mol_type = other DNA
gggttcagcgtcagtagttattac

SEQ ID NO: 290 - DNA, 21 bp, Homo sapiens
  source 1..21: mol_type = other DNA
acttataccgatgggagcaca

SEQ ID NO: 291 - DNA, 42 bp, Homo sapiens
  source 1..42: mol_type = other DNA
gcgagagaaggagatggctacaattcgggaggtcttgactac

SEQ ID NO: 292 - DNA, 24 bp, Homo sapiens
  source 1..24: mol_type = other DNA
ggaggcaccttcagcagctatgct

SEQ ID NO: 293 - DNA, 24 bp, Homo sapiens
  source 1..24: mol_type = other DNA
atcatccctatctttggtacagca

SEQ ID NO: 294 - DNA, 39 bp, Homo sapiens
  source 1..39: mol_type = other DNA
gcggtctcgatttttggagtggttattccactagactac

SEQ ID NO: 295 - DNA, 24 bp, Homo sapiens
  source 1..24: mol_type = other DNA
ggaggcaccttcagcagctatgct

SEQ ID NO: 296 - DNA, 24 bp, Homo sapiens
  source 1..24: mol_type = other DNA
atcatccctatctttggtacagca

SEQ ID NO: 297 - DNA, 36 bp, Homo sapiens
  source 1..36: mol_type = other DNA
gcgagaagtggttactataatgatgcttttgatatc

SEQ ID NO: 298 - DNA, 24 bp, Homo sapiens
  source 1..24: mol_type = other DNA
ggttacacctttaccagctatggt

SEQ ID NO: 299 - DNA, 24 bp, Homo sapiens
  source 1..24: mol_type = other DNA
atcagcgcttacaatggtaacaca

SEQ ID NO: 300 - DNA, 51 bp, Homo sapiens
  source 1..51: mol_type = other DNA
gcgagagatcaaaagttactacatattctctactactacggtatggacgtc

SEQ ID NO: 301 - DNA, 24 bp, Homo sapiens
  source 1..24: mol_type = other DNA
ggatacagctttaccagctactgg

SEQ ID NO: 302 - DNA, 24 bp, Homo sapiens
  source 1..24: mol_type = other DNA
attgatcctagtgactcttatacc

SEQ ID NO: 303 - DNA, 36 bp, Homo sapiens
  source 1..36: mol_type = other DNA
gcgagggcccgcacctggtttgatgcttttgatatc

SEQ ID NO: 304 - DNA, 24 bp, Homo sapiens
  source 1..24: mol_type = other DNA
ggttacacctttaccagctatggt

SEQ ID NO: 305 - DNA, 24 bp, Homo sapiens
  source 1..24: mol_type = other DNA
atcagcgcttacaatggtaacaca

SEQ ID NO: 306 - DNA, 36 bp, Homo sapiens
  source 1..36: mol_type = other DNA
gcgagagttgggtactactactacggtatggacgtc

SEQ ID NO: 307 - DNA, 18 bp, Homo sapiens
  source 1..18: mol_type = other DNA
aaattgcgggaagatttg

SEQ ID NO: 308 - DNA, 30 bp, Homo sapiens
  source 1..30: mol_type = other DNA
caggcgtgggacaatagtgtcgattatgtc

SEQ ID NO: 309 - DNA, 18 bp, Homo sapiens
  source 1..18: mol_type = other DNA
cagaatattaataattgg

SEQ ID NO: 310 - DNA, 39 bp, Homo sapiens
  source 1..39: mol_type = other DNA
caacaatataagaattatccgtacagtaatctgtacact

SEQ ID NO: 311 - DNA, 18 bp, Homo sapiens
  source 1..18: mol_type = other DNA
cagagcattagcagctat

SEQ ID NO: 312 - DNA, 27 bp, Homo sapiens
  source 1..27: mol_type = other DNA
caacagagttacagtacccctcccact

SEQ ID NO: 313 - DNA, 24 bp, Homo sapiens
  source 1..24: mol_type = other DNA
aactccaacatcggatcttatatt

SEQ ID NO: 314 - DNA, 33 bp, Homo sapiens
  source 1..33: mol_type = other DNA
gcggcatgggataacatcctgaataattatgtc

SEQ ID NO: 315 - DNA, 18 bp, Homo sapiens
  source 1..18: mol_type = other DNA
cagagcattggaacctat

SEQ ID NO: 316 - DNA, 27 bp, Homo sapiens
  source 1..27: mol_type = other DNA
caacagacttacatcacccctcggacg

SEQ ID NO: 317 - DNA, 18 bp, Homo sapiens
  source 1..18: mol_type = other DNA
aacattggaaggaaaagt

SEQ ID NO: 318 - DNA, 33 bp, Homo sapiens
  source 1..33: mol_type = other DNA
caggtgtgggatagtagtagtgcttcgggagtg

SEQ ID NO: 319 - DNA, 27 bp, Homo sapiens
  source 1..27: mol_type = other DNA
aacagtgacattggtggttataactat

SEQ ID NO: 320 - DNA, 30 bp, Homo sapiens
  source 1..30: mol_type = other DNA
agctcatatacaagctccagcactctcgta

SEQ ID NO: 321 - DNA, 33 bp, Homo sapiens
  source 1..33: mol_type = other DNA
cagagcctcctgcatagtaatggaaacaactat

SEQ ID NO: 322 - DNA, 27 bp, Homo sapiens
  source 1..27: mol_type = other DNA
atgcaagctctacaaactgcttacact

SEQ ID NO: 323 - DNA, 24 bp, Homo sapiens
  source 1..24: mol_type = other DNA
agctccaacatcggacgttattct

SEQ ID NO: 324 - DNA, 33 bp, Homo sapiens
  source 1..33: mol_type = other DNA
gcagcatgggatgacagcctgaaaggttgggtc

SEQ ID NO: 325 - DNA, 27 bp, Homo sapiens
  source 1..27: mol_type = other DNA
agctccaacatcggggcaggctttgac

SEQ ID NO: 326 - DNA, 33 bp, Homo sapiens
  source 1..33: mol_type = other DNA
caatcctatgacaggagcctgagtggttatgtc

SEQ ID NO: 327 - DNA, 75 bp, Homo sapiens
  source 1..75: mol_type = other DNA
caggtgcagctggtgcagtccggagcagaggtgaaaaagcccggggagtctctgaggatctcctgtaagggttct

SEQ ID NO: 328 - DNA, 51 bp, Homo sapiens
  source 1..51: mol_type = other DNA
atcagctgggtgcgccagatgcccgggaaaggcctggagtggatggggagg

SEQ ID NO: 329 - DNA, 114 bp, Homo sapiens
  source 1..114: mol_type = other DNA
aactacagcccgtccttccaaggccacgtcaccatctcagctgacaagtccatcagcactgcctacctgcagtggagcagcctgaaggcctcggacaccgccatgtattactgt

SEQ ID NO: 330 - DNA, 33 bp, Homo sapiens
  source 1..33: mol_type = other DNA
tggggccagggaaccctggtcaccgtctcctca

SEQ ID NO: 331 - DNA, 75 bp, Homo sapiens
  source 1..75: mol_type = other DNA
caggtgcagctggtgcagtctggggctgaggtgaagaagcctgggtcctcgatgaaggtctcctgcaaggcttct

SEQ ID NO: 332 - DNA, 51 bp, Homo sapiens
  source 1..51: mol_type = other DNA
atcagctgggtgcgacaggcccctggacaaggacttgagtggatgggaggg

SEQ ID NO: 333 - DNA, 114 bp, Homo sapiens
  source 1..114: mol_type = other DNA
aagtacgcacagaagttccagggcagagtcacgattaccgcggacgaatccacgaccacagcctacatggagctgagcagcctgagctctgaggacacggccgtgtattactgt

SEQ ID NO: 334 - DNA, 33 bp, Homo sapiens
  source 1..33: mol_type = other DNA
tggggccaagggacaatggtcaccgtctcctca

SEQ ID NO: 335 - DNA, 75 bp, Homo sapiens
  source 1..75: mol_type = other DNA
caggtgcagctggtgcagtctggagctgaggtgaagaagcctggggcctcagtgaaggtctcctgcaaggcttct

SEQ ID NO: 336 - DNA, 51 bp, Homo sapiens
  source 1..51: mol_type = other DNA
atcagctgggtgcgacaggcccctggacaagggcttgagtggatgggatgg

SEQ ID NO: 337 - DNA, 114 bp, Homo sapiens
  source 1..114: mol_type = other DNA
aactatgcacagaagctccagggcagagtcaccatgaccacagacacatccacgagcacagcctacatggagctgaggagcctgagatctgacgacacggccgtgtattactgt

SEQ ID NO: 338 - DNA, 33 bp, Homo sapiens
  source 1..33: mol_type = other DNA
tggggccaagggaccacggtcaccgtctcctca

SEQ ID NO: 339 - DNA, 75 bp, Homo sapiens; identical to SEQ ID NO: 327
  source 1..75: mol_type = other DNA

SEQ ID NO: 340 - DNA, 51 bp, Homo sapiens; identical to SEQ ID NO: 328
  source 1..51: mol_type = other DNA

SEQ ID NO: 341 - DNA, 114 bp, Homo sapiens; identical to SEQ ID NO: 329
  source 1..114: mol_type = other DNA

SEQ ID NO: 342 - DNA, 33 bp, Homo sapiens
  source 1..33: mol_type = other DNA
tggggccaagggacaatggtcaccgtctcttca

SEQ ID NO: 343 - DNA, 75 bp, Homo sapiens
  source 1..75: mol_type = other DNA
gaggtgcagctgttggagtctggaggaggcttgatccagcctggggggtccctgagactctcctgtgcagcctct

SEQ ID NO: 344 - DNA, 51 bp, Homo sapiens
  source 1..51: mol_type = other DNA
atgaactgggtccgccaggctccagggaagggactggagtgggtctcaaat

SEQ ID NO: 345 - DNA, 114 bp, Homo sapiens
  source 1..114: mol_type = other DNA
tcttatgcagactccgtggggggccgattcagcgtctcaagagacaattccaagaacacgttgcatcttcaaatgaacagcctgagagtcgaagacacggccgtgtattactgt

SEQ ID NO: 346 - DNA, 33 bp, Homo sapiens
  source 1..33: mol_type = other DNA
tggggccagggaaccctggtcaccgtctcttca

SEQ ID NO: 347 - DNA, 75 bp, Homo sapiens
  source 1..75: mol_type = other DNA
ccggtgcttctggtgcagtctggggctgaggtgaagaagcctgggtcctcggtgaaggtctcctgcaaggcttct

SEQ ID NO: 348 - DNA, 51 bp, Homo sapiens
  source 1..51: mol_type = other DNA
atcagctgggtgcgacaggcccctggacaagggcttgagtggatgggaggg

SEQ ID NO: 349 - DNA, 114 bp, Homo sapiens
  source 1..114: mol_type = other DNA
aactacgcacagaagttccagggcagagtcacgattaccgcggacgaatccacgagcacagcctacatggagctgagcagcctgagatctgaggacacggccgtgtattactgt

SEQ ID NO: 350 - DNA, 33 bp, Homo sapiens
  source 1..33: mol_type = other DNA
tggggccagggaaccctggtcatcgtctcctca

SEQ ID NO: 351 - DNA, 75 bp, Homo sapiens
  source 1..75: mol_type = other DNA
cagcctgtgctgactcagccaccctcagtgtccgtgtccccaggacagacagcccgcatcacctgctctggagat

SEQ ID NO: 352 - DNA, 51 bp, Homo sapiens
  source 1..51: mol_type = other DNA
acttcctggtatcagcagaagccaggccagtcccctgtgctggtcatctat

SEQ ID NO: 353 - DNA, 108 bp, Homo sapiens
  source 1..108: mol_type = other DNA
aagcggccctcagggatccctgcgcgattttctggctccatctctgggagcacagccactctgaccatcagcgggactcaggctgtggatgaggctgactattactgt

SEQ ID NO: 354 - DNA, 30 bp, Homo sapiens
  source 1..30: mol_type = other DNA
ttcggaactgggaccaaggtcaccgtccta

SEQ ID NO: 355 - DNA, 78 bp, Homo sapiens
  source 1..78: mol_type = other DNA
gacatcgtgatgacccagtctccttccaccctgtctgcatctgtgggagacagagtcaccatcacttgccgggccagt

SEQ ID NO: 356 - DNA, 51 bp, Homo sapiens
  source 1..51: mol_type = other DNA
ttggcctggtatcagcagaaaccaggaaaagcccctaaactcctgatctat

SEQ ID NO: 357 - DNA, 108 bp, Homo sapiens
  source 1..108: mol_type = other DNA
actgcagagagtggggtcccgtcaagattcagcggctctggatttgggacagaattcactctcaccatcagcagcctgcagcctgatgattttgcatcttattactgc

SEQ ID NO: 358 - DNA, 30 bp, Homo sapiens
  source 1..30: mol_type = other DNA
tttggccaggggaccaaagtggatatcaaa

SEQ ID NO: 359 - DNA, 78 bp, Homo sapiens
  source 1..78: mol_type = other DNA
gacatccagatgacccagtctccatcctccctgtctgcatctgtaggagacagagtcaccatcacttgccgggcaagt

SEQ ID NO: 360 - DNA, 51 bp, Homo sapiens
  source 1..51: mol_type = other DNA
ttaaattggtatcagcagaaaccagggaaagcccctaagctcctgatctat

SEQ ID NO: 361 - DNA, 108 bp, Homo sapiens
  source 1..108: mol_type = other DNA
agtttgcaaagtggggtcccatcaaggttcagtggcagtggatctgggacagatttcactctcaccatcagcagtctgcaacctgaagattttgcaacttactactgt

SEQ ID NO: 362 - DNA, 30 bp, Homo sapiens
  source 1..30: mol_type = other DNA
ttcggcggagggaccaaagtggatatcaaa

SEQ ID NO: 363 - DNA, 75 bp, Homo sapiens
  source 1..75: mol_type = other DNA
caggctgtggtgactcagccaccctcagtgtctgggacccccgggcagagggtcaccatctcttgttctggaagc

SEQ ID NO: 364 - DNA, 51 bp, Homo sapiens
  source 1..51: mol_type = other DNA
gtaaattggtatcagcaactcccaggaacggcccccaagctcctcatctgg

SEQ ID NO: 365 - DNA, 108 bp, Homo sapiens
  source 1..108: mol_type = other DNA
cggcggccctcaggggtccctgaccgagtctctgcctccaagtctggtacctcagcctccctggccatcagtgggctccagtctgaggatgagggtgattattactgt

SEQ ID NO: 366 - DNA, 30 bp, Homo sapiens
  source 1..30: mol_type = other DNA
ttcggacctgggacccagctgaccgtccta

SEQ ID NO: 367 - DNA, 78 bp, Homo sapiens
  source 1..78: mol_type = other DNA
gacatccagttgacccagtctccagcctccctgtctgcctctttaggagacagagtcaccattacttgccgggcgagt

SEQ ID NO: 368 - DNA, 51 bp, Homo sapiens
  source 1..51: mol_type = other DNA
ctgaattggtatcagcagagaccagggcaagcccctaaactcctgatcttt

SEQ ID NO: 369 - DNA, 108 bp, Homo sapiens
  source 1..108: mol_type = other DNA
agtttgcaaagtggggtcccatcaagattcagtgggagtggatctgggacagatttcactctcaccatcagcagtctgcaacccgaagatttggcaacttactactgt

SEQ ID NO: 370 - DNA, 30 bp, Homo sapiens
  source 1..30: mol_type = other DNA
ttcggccaagggaccaaagtggaaatcaaa

SEQ ID NO: 371 - DNA, 75 bp, Homo sapiens
  source 1..75: mol_type = other DNA
cagcctgtgctgactcagtcaccctcagtgtcactggccccaggacagacggccaagattacctgtgggggaacc

SEQ ID NO: 372 - DNA, 51 bp, Homo sapiens
  source 1..51: mol_type = other DNA
gtgcactggtaccagcagaagccgggccaggcccccgtgatggtcatttat

SEQ ID NO: 373 - DNA, 108 bp, Homo sapiens
  source 1..108: mol_type = other DNA
gaccggccctcagggatccctgagcgattcgctggctccaattctgggaacacggccaccctgaccatcagcagggtcgaagccggggatgaggccgtctattactgt

SEQ ID NO: 374 - DNA, 30 bp, Homo sapiens
  source 1..30: mol_type = other DNA
ttcggcggagggaccaaggtcaccgtccta

SEQ ID NO: 375 - DNA, 75 bp, Homo sapiens
  source 1..75: mol_type = other DNA
cagtctgccctgactcagcctgcctccgtgtctgggtctcctggacagtcgatcaccatctcctgcactggaacc

SEQ ID NO: 376 - DNA, 51 bp, Homo sapiens
  source 1..51: mol_type = other DNA
gtctcctggtaccaacagcacccaggcaaagcccccaaactcatgatttat

SEQ ID NO: 377 - DNA, 108 bp, Homo sapiens
  source 1..108: mol_type = other DNA
aatcggccctcaggggtttctaatcgcttctctggctccaagtctggcaacacggcctccctgaccatctctgggctccaggctgaggacgaggctcattattactgc

SEQ ID NO: 378 - DNA, 30 bp, Homo sapiens
  source 1..30: mol_type = other DNA
ttcggcggagggaccaaggtnaccgtccta

SEQ ID NO: 379 - DNA, 78 bp, Homo sapiens
  source 1..78: mol_type = other DNA
gatattgtgatgactcagtctccactctccctgtccgtcacccctggagagccggcctccatctcctgcaggtctagt

SEQ ID NO: 380 - DNA, 51 bp, Homo sapiens
  source 1..51: mol_type = other DNA
ttggattggtacctgcagaagccagggcagtctccacagctcctgatctat

SEQ ID NO: 381 - DNA, 108 bp, Homo sapiens
  source 1..108: mol_type = other DNA
aatcgggcctccggggtccctgacaggttcagtggcagtggatcaggcacagatttttcactgaaaatcaacaaagtggaggctgaggatgttggggtttattactgc

SEQ ID NO: 382 - DNA, 30 bp, Homo sapiens
  source 1..30: mol_type = other DNA
tttggccaggggaccaagctggagatcaaa

SEQ ID NO: 383 - DNA, 75 bp, Homo sapiens
  source 1..75: mol_type = other DNA
caggctgtggtgactcagccaccctcagcgtctgggacccccggccagagggtcaccatctcttgttctggaggc

SEQ ID NO: 384 - DNA, 51 bp, Homo sapiens
  source 1..51: mol_type = other DNA
gtaaactggtaccaacagctcccaggaacggcccccaaactcctcatcaat

SEQ ID NO: 385 - DNA, 108 bp, Homo sapiens
  source 1..108: mol_type = other DNA
cagcggccctcaggggtccctgaccgattctctggctccaagtctggcacctcagcctccctggccatcagtgggctccagtctgaggatgaggctgattatcactgt

SEQ ID NO: 386 - DNA, 30 bp, Homo sapiens; identical to SEQ ID NO: 374
  source 1..30: mol_type = other DNA

SEQ ID NO: 387 - DNA, 75 bp, Homo sapiens
  source 1..75: mol_type = other DNA
cagtctgtgttgacgcagccgccctcagtgtctggggccccagggcagagggtcaccatctcctgcactgggagc

SEQ ID NO: 388 - DNA, 51 bp, Homo sapiens
  source 1..51: mol_type = other DNA
gtacactggtaccagcagcttccaggaacagcccccaaactcctcatctat

SEQ ID NO: 389 - DNA, 108 bp, Homo sapiens
  source 1..108: mol_type = other DNA
aatcggccctcaggggtccctgaccgattctctgcctccaagtctggcacctcagcctccctggccatcactgggctccaggctgaggatgaggctgattattactgc

SEQ ID NO: 390 - DNA, 30 bp, Homo sapiens
  source 1..30: mol_type = other DNA
ttcggaagtgggaccaaggtcaccgtccta

SEQ ID NO: 391 - DNA, 375 bp, Homo sapiens
  source 1..375: mol_type = other DNA
caggtgcagctggtgcagtccggagcagaggtgaaaaagcccggggagtctctgaggatctcctgtaagggttctggatacagctttaccagctactggatcagctgggtgcgccagatgcccgggaaaggcctggagtggatggggaggattgatcctagtgactcttataccaactacagcccgtccttccaaggccacgtcaccatctcagctgacaagtccatcagcactgcctacctgcagtggagcagcctgaaggcctcggacaccgccatgtattactgtgcgagacatcccctagcatattgtggtggtgactgccaacggggctttgactactggggccagggaaccctggtcaccgtctcctca

SEQ ID NO: 392 - DNA, 357 bp, Homo sapiens
  source 1..357: mol_type = other DNA
caggtgcagctggtgcagtctggggctgaggtgaagaagcctgggtcctcgatgaaggtctcctgcaaggcttctggaggcaccttcagcagttatgctatcagctgggtgcgacaggcccctggacaaggacttgagtggatgggagggatcatccctatctttggtacagcaaagtacgcacagaagttccagggcagagtcacgattaccgcggacgaatccacgaccacagcctacatggagctgagcagcctgagctctgaggacacggccgtgtattactgtgcgagaagtcttggctggtactatgcttttgatatctggggccaagggacaatggtcaccgtctcctca

SEQ ID NO: 393 - DNA, 375 bp, Homo sapiens
  source 1..375: mol_type = other DNA
caggtgcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagagatagtagaatgtcactggagtatgctcccgggtacggaatggacgtctggggccaagggaccacggtcaccgtctcctca

SEQ ID NO: 394 - DNA, 369 bp, Homo sapiens
  source 1..369: mol_type = other DNA
caggtgcagctggtgcagtccggagcagaggtgaaaaagcccggggagtctctgaggatctcctgtaagggttctggatacagctttaccagctactggatcagctgggtgcgccagatgcccgggaaaggcctggagtggatggggaggattgatcctagtgactcttataccaactacagcccgtccttccaaggccacgtcaccatctcagctgacaagtccatcagcactgcctacctgcagtggagcagcctgaaggcctcggacaccgccatgtattactgtgcgagacatcccccccgatcaacgcggggtgactacggtatggacgtctggggccaagggacaatggtcaccgtctcttca

SEQ ID NO: 395 - DNA, 360 bp, Homo sapiens
  source 1..360: mol_type = other DNA
gaggtgcagctgttggagtctggaggaggcttgatccagcctggggggtccctgagactctcctgtgcagcctctgggttcagcgtcagtagttattacatgaactgggtccgccaggctccagggaagggactggagtgggtctcaaatacttataccgatgggagcacatcttatgcagactccgtggggggccgattcagcgtctcaagagacaattccaagaacacgttgcatcttcaaatgaacagcctgagagtcgaagacacggccgtgtattactgtgcgagagaaggagatggctacaattcgggaggtcttgactactggggccagggaaccctggtcaccgtctcttca

SEQ ID NO: 396 - DNA, 360 bp, Homo sapiens
  source 1..360: mol_type = other DNA
ccggtgcttctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcggtctcgatttttggagtggttattccactagactactggggccagggaaccctggtcatcgtctcctca

SEQ ID NO: 397 - DNA, 357 bp, Homo sapiens
  source 1..357: mol_type = other DNA
caggttcagcttgtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagaagtggttactataatgatgcttttgatatctggggccaagggacaatggtcaccgtctcttca

SEQ ID NO: 398 - DNA, 372 bp, Homo sapiens
  source 1..372: mol_type = other DNA
caagtgcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagagatcaaaagttactacatattctctactactacggtatggacgtctggggccaagggaccacggtcatcgtctcctca

SEQ ID NO: 399 - DNA, 357 bp, Homo sapiens
  source 1..357: mol_type = other DNA
caggttcagcttgtgcagtccggagcagaggtgaaaaagcccggggagtctctgaggatctcctgtaagggttctggatacagctttaccagctactggatcagctgggtgcgccagatgcccgggaaaggcctggagtggatggggaggattgatcctagtgactcttataccaactacagcccgtccttccaaggccacgtcaccatctcagctgacaagtccatcagcactgcctacctgcagtggagcagcctgaaggcctcggacaccgccatatattactgtgcgagggcccgcacctggtttgatgcttttgatatctggggccaagggaccacggtcaccgtctcctca

SEQ ID NO: 400 - DNA, 321 bp, Homo sapiens
  source 1..321: mol_type = other DNA
cagcctgtgctgactcagccaccctcagtgtccgtgtccccaggacagacagcccgcatcacctgctctggagataaattgcgggaagatttgacttcctggtatcagcagaagccaggccagtcccctgtgctggtcatctatcaagattccaagcggccctcagggatccctgcgcgattttctggctccatctctgggagcacagccactctgaccatcagcgggactcaggctgtggatgaggctgactattactgtcaggcgtgggacaatagtgtcgattatgtcttcggaactgggaccaaggtcaccgtccta

SEQ ID NO: 401 - DNA, 333 bp, Homo sapiens
  source 1..333: mol_type = other DNA
gacatcgtgatgacccagtctccttccaccctgtctgcatctgtgggagacagagtcaccatcacttgccgggccagtcagaatattaataattggttggcctggtatcagcagaaaccaggaaaagcccctaaactcctgatctataaggcatccactgcagagagtggggtcccgtcaagattcagcggctctggatttgggacagaattcactctcaccatcagcagcctgcagcctgatgattttgcatcttattactgccaacaatataagaattatccgtacagtaatctgtacacttttggccaggggaccaaagtggatatcaaa

SEQ ID NO: 402 - DNA, 321 bp, Homo sapiens
  source 1..321: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctcccactttcggcggagggaccaaagtggatatcaaa

SEQ ID NO: 403 - DNA, 330 bp, Homo sapiens
  source 1..330: mol_type = other DNA
caggctgtggtgactcagccaccctcagtgtctgggacccccgggcagagggtcaccatctcttgttctggaagcaactccaacatcggatcttatattgtaaattggtatcagcaactcccaggaacggcccccaagctcctcatctggggtaatgatcggcggccctcaggggtccctgaccgagtctctgcctccaagtctggtacctcagcctccctggccatcagtgggctccagtctgaggatgagggtgattattactgtgcggcatgggataacatcctgaataattatgtcttcggacctgggacccagctgaccgtccta

SEQ ID NO: 404 - DNA, 321 bp, Homo sapiens
  source 1..321: mol_type = other DNA
gacatccagttgacccagtctccagcctccctgtctgcctctttaggagacagagtcaccattacttgccgggcgagtcagagcattggaacctatctgaattggtatcagcagagaccagggcaagcccctaaactcctgatctttggtacatccagtttgcaaagtggggtcccatcaagattcagtgggagtggatctgggacagatttcactctcaccatcagcagtctgcaacccgaagatttggcaacttactactgtcaacagacttacatcacccctcggacgttcggccaagggaccaaagtggaaatcaaa

SEQ ID NO: 405 - DNA, 324 bp, Homo sapiens
  source 1..324: mol_type = other DNA
cagcctgtgctgactcagtcaccctcagtgtcactggccccaggacagacggccaagattacctgtgggggaaccaacattggaaggaaaagtgtgcactggtaccagcagaagccgggccaggcccccgtgatggtcatttattatgatagcgaccggccctcagggatccctgagcgattcgctggctccaattctgggaacacggccaccctgaccatcagcagggtcgaagccggggatgaggccgtctattactgtcaggtgtgggatagtagtagtgcttcgggagtgttcggcggagggaccaaggtcaccgtccta

SEQ ID NO: 406 - DNA, 330 bp, Homo sapiens
  source 1..330: mol_type = other DNA
cagtctgccctgactcagcctgcctccgtgtctgggtctcctggacagtcgatcaccatctcctgcactggaaccaacagtgacattggtggttataactatgtctcctggtaccaacagcacccaggcaaagcccccaaactcatgatttatgaggtcagtaatcggccctcaggggtttctaatcgcttctctggctccaagtctggcaacacggcctccctgaccatctctgggctccaggctgaggacgaggctcattattactgcagctcatatacaagctccagcactctcgtattcggcggagggaccaaggtnaccgtccta

SEQ ID NO: 407 - DNA, 336 bp, Homo sapiens
  source 1..336: mol_type = other DNA
gatattgtgatgactcagtctccactctccctgtccgtcacccctggagagccggcctccatctcctgcaggtctagtcagagcctcctgcatagtaatggaaacaactatttggattggtacctgcagaagccagggcagtctccacagctcctgatctatttgggttctaatcgggcctccggggtccctgacaggttcagtggcagtggatcaggcacagatttttcactgaaaatcaacaaagtggaggctgaggatgttggggtttattactgcatgcaagctctacaaactgcttacacttttggccaggggaccaagctggagatcaaa

SEQ ID NO: 408 - DNA, 330 bp, Homo sapiens
  source 1..330: mol_type = other DNA
caggctgtggtgactcagccaccctcagcgtctgggacccccggccagagggtcaccatctcttgttctggaggcagctccaacatcggacgttattctgtaaactggtaccaacagctcccaggaacggcccccaaactcctcatcaatagtaatattcagcggccctcaggggtccctgaccgattctctggctccaagtctggcacctcagcctccctggccatcagtgggctccagtctgaggatgaggctgattatcactgtgcagcatgggatgacagcctgaaaggttgggtcttcggcggagggaccaaggtcaccgtccta

SEQ ID NO: 409 - DNA, 333 bp, Homo sapiens
  source 1..333: mol_type = other DNA
cagtctgtgttgacgcagccgccctcagtgtctggggccccagggcagagggtcaccatctcctgcactgggagcagctccaacatcggggcaggctttgacgtacactggtaccagcagcttccaggaacagcccccaaactcctcatctatgctaatagcaatcggccctcaggggtccctgaccgattctctgcctccaagtctggcacctcagcctccctggccatcactgggctccaggctgaggatgaggctgattattactgccaatcctatgacaggagcctgagtggttatgtcttcggaagtgggaccaaggtcaccgtccta

SEQ ID NO: 410 - DNA, 357 bp, Homo sapiens
  source 1..357: mol_type = other DNA
caggtgcagctggtgcaatctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagagttgggtactactactacggtatggacgtctggggccaagggaccacggtcaccgtctcctca